(19) United States
(12) Patent Application Publication
(10) Pub. No.: US 2026/0354323 A1
(43) Pub. Date: Nov. 12, 2026

(54) IMPROVED WHITE CORN

(71) Applicant: PIONEER HI-BRED INTERNATIONAL, INC., JOHNSTON, IA (US)

(72) Inventors: KEVIN A. FENGLER, CLIVE, IA (US); SIVA S. AMMIRAJU JETTY, JOHNSTON, IA (US); ZACHARY GLEN JONES, FISHERS, IN (US); JOSHUA MICHAEL SHENDELMAN, ANKENY, IA (US)

(73) Assignee: PIONEER HI-BRED INTERNATIONAL, INC., JOHNSTON, IA (US)

(21) Appl. No.: 19/130,785

(22) PCT Filed: Nov. 14, 2023
(86) PCT No.: PCT/US2023/079622

§ 371 Date: May 16, 2025

Related U.S. Application Data
(60) Provisional application No. 63/384,287, filed on Nov. 18, 2022.

(51) Int. Cl. A01H 6/46; A01H 1/04; A01H 5/10

(52) U.S. Cl. CPC A01H 6/4684; A01H 1/045; A01H 5/10

(57) ABSTRACT

Methods for producing white corn seeds and products and efficient breeding methods are provided. Maize plant populations which contain alleles resulting in whiter kernels, alone or in combination with each other are produced using genomic markers in a plant breeding program. Methods for converting an elite maize plant with yellow kernels to an equivalent elite maize plant with white kernels include selection for kernel whiteness and backcrossing or double haploid creation.

### REFERENCE TO SEQUENCE LISTING SUBMITTED ELECTRONICALLY

The official copy of the sequence listing is submitted electronically via Patent Center as an XML formatted sequence listing having the file name “9214-US-PSP_SequenceListing.xml” created on Nov. 14, 2022 and having a size of 299,192 bytes. The sequence listing comprised in this XML formatted document is part of the specification and is herein incorporated by reference in its entirety.

### BACKGROUND

A goal of plant breeding is to combine various desirable traits in a single plant. For field crops such as corn, these traits can include general agronomic traits, such as greater yield and better agronomic quality, as well as specific agronomic traits such as white corn kernels. White corn is considered a specialty corn and accounts for around 1% of US corn acres.

In plant breeding, desirable loci can be identified and selected for to generate plants that carry desirable agronomic traits. Introgression of desirable loci into commercially available plant varieties can be achieved using marker-assisted selection or marker-assisted breeding, in which one or more molecular markers are used for the identification and selection of progeny plants containing the one or more loci that encode or effect the desired traits.

### SUMMARY

Provided are methods of producing a corn plant having non-yellow kernels and comprising a bn7 allele, which include the steps of genotyping a plurality of corn plants or corn seeds having in their pedigree at least one non-yellow corn variety at a bn7 marker locus associated with kernel color that is within 5 CM of SEQ ID NO. 70, selecting one or more corn plants or corn seeds comprising a bn7 allele contributing to whiter kernels and crossing selected corn plants or plants grown from the selected seeds with other plants comprising the bn7 allele or self-pollinating the selected plants or plants grown from the selected corn seeds to produce a plant having non-yellow kernels and comprising the bn7 allele. The bn7 marker locus can be between for example SEQ ID NO: 118 and SEQ ID NO: 206, 148-186 SEQ ID NO: 99 and SEQ ID NO: 104, inclusive.

In some embodiments, a method of producing a maize plant having non-yellow kernels comprises genotyping a nucleic acid isolated from a maize plant for the presence of a marker located within a chromosomal interval defined by and comprising SEQ ID NO:115 and SEQ ID NO: 208, the interval comprising a C corresponding to position 201 of SEQ ID NO: 157, selecting a first plant on the basis of the presence of the marker genotyped in (a); crossing the first plant with a second plant to produce progeny seed and selecting a progeny seed or plant grown therefrom comprising the C corresponding to position 201 of SEQ ID NO: 157, thereby producing a plant having non-yellow kernels.

In some embodiments, the selected corn plant comprises a G at position corresponding to 201 of SEQ ID NO: 148, a G at position corresponding to 201 of SEQ ID NO: 149, a C at position corresponding to 201 of SEQ ID NO: 150, a C at position corresponding to 201 of SEQ ID NO: 151, a C at position corresponding to 201 of SEQ ID NO: 152, a T at position corresponding to 201 of SEQ ID NO: 153, a G at position corresponding to 201 of SEQ ID NO: 154, a G at position corresponding to 201 of SEQ ID NO: 155, a T at position corresponding to 201 of SEQ ID NO: 156, a G at position corresponding to 201 of SEQ ID NO: 158, a C at position corresponding to 201 of SEQ ID NO: 159, a G at position corresponding to 201 of SEQ ID NO: 160, a G at position corresponding to 201 of SEQ ID NO: 161, a G at position corresponding to 201 of SEQ ID NO: 162, an A at position corresponding to 201 of SEQ ID NO: 163, an A at position corresponding to 201 of SEQ ID NO: 164, a G at position corresponding to 201 of SEQ ID NO: 165, a T at position corresponding to 201 of SEQ ID NO: 166, a C at position corresponding to 201 of SEQ ID NO: 167, a G at position corresponding to 201 of SEQ ID NO: 168, a G at position corresponding to 201 of SEQ ID NO: 169, a C at position corresponding to 201 of SEQ ID NO: 170, a T at position corresponding to 201 of SEQ ID NO: 171, a T at position corresponding to 201 of SEQ ID NO: 172, an A at position corresponding to 201 of SEQ ID NO: 173, an A at position corresponding to 201 of SEQ ID NO: 174, a T at position corresponding to 201 of SEQ ID NO: 175, a G at position corresponding to 201 of SEQ ID NO: 176, a C at position corresponding to 201 of SEQ ID NO: 177, a C at position corresponding to 201 of SEQ ID NO: 178, an A at position corresponding to 201 of SEQ ID NO: 179, an A at position corresponding to 201 of SEQ ID NO: 180, a G at position corresponding to 201 of SEQ ID NO: 181, a T at position corresponding to 201 of SEQ ID NO: 182, a C at position corresponding to 201 of SEQ ID NO: 183, an AG at position corresponding to 201 of SEQ ID NO: 184, a T at position corresponding to 201 of SEQ ID NO: 185, a T at position corresponding to 201 of SEQ ID NO: 186, a C at position corresponding to position 51 of SEQ ID NO: 99, or a C at position corresponding to position 51 of SEQ ID NO: 104.

In some embodiments, the method further includes genotyping one or more additional loci. For example, a y1 marker locus associated with at least one y1 recessive white allele for a polynucleotide encoding a polypeptide having at least 95% identity with SEQ ID NO: 110 can be genotyped and corn plants or corn seeds are selected comprising the bn7 white allele and at least one y1 recessive white allele. Genotyping the y1 marker locus can include detecting the zygosity state of the y1 locus and selecting a heterozygous Y1/y1 allele, a homozygous y1/y1 allele or a combination thereof. Plants selected for y1 recessive allele can comprise SEQ ID NO: 5 or have a C nucleotide at the position that corresponds to position 301 of SEQ ID NO: 19. For example, a Ccd1 marker locus, such as comprising SEQ ID NO: 29, associated with Wc1 encoding a polynucleotide encoding a polypeptide having at least 95% identity with SEQ ID NO: 112 can be genotyped and corn plants or corn seeds are selected comprising the bn7 white allele and at least one, two or three or more copies of Ccd1. For example, a P1 marker locus for white cob can be genotyped, and optionally the plant selected may comprise P1-ww for white cob, white pericarp.

In some embodiments, a color sorter is used to separate the seeds on the basis of color. Selected plants can contain white kernels, a white cob or a combination thereof.

In some embodiments, the methods include selecting plants that are homozygous for bn7, homozygous for y1, comprises P1-ww for white cob, white pericarp, have at least three copies of Ccd1 or any combination thereof.

In some embodiments, the methods include a genotyping step which comprises assaying a SNP marker, use of a polynucleotide probe, detecting a haplotype or a combination thereof.

Provided are methods for introgressing one or more alleles contributing to kernel whiteness into an elite yellow corn plant, by genotyping at a bn7 marker locus within 5 CM of SEQ ID NO. 70 and associated with kernel color seeds taken from a population of seeds derived from a cross of an elite yellow corn plant with a non-yellow corn plant, selecting progeny plants comprising at least one of: a C corresponding to position 51 of SEQ ID NO: 99, a C corresponding to position 51 of SEQ ID NO: 104, a G at position corresponding to 201 of SEQ ID NO: 148, a G at position corresponding to 201 of SEQ ID NO: 149, a C at position corresponding to 201 of SEQ ID NO: 150, a C at position corresponding to 201 of SEQ ID NO: 151, a C at position corresponding to 201 of SEQ ID NO: 152, a T at position corresponding to 201 of SEQ ID NO: 153, a G at position corresponding to 201 of SEQ ID NO: 154, a G at position corresponding to 201 of SEQ ID NO: 155, a T at position corresponding to 201 of SEQ ID NO: 156, a C at position corresponding to 201 of SEQ ID NO: 157, a G at position corresponding to 201 of SEQ ID NO: 158, a C at position corresponding to 201 of SEQ ID NO: 159, a G at position corresponding to 201 of SEQ ID NO: 160, a G at position corresponding to 201 of SEQ ID NO: 161, a G at position corresponding to 201 of SEQ ID NO: 162, an A at position corresponding to 201 of SEQ ID NO: 163, an A at position corresponding to 201 of SEQ ID NO: 164, a G at position corresponding to 201 of SEQ ID NO: 165, a T at position corresponding to 201 of SEQ ID NO: 166, a C at position corresponding to 201 of SEQ ID NO: 167, a G at position corresponding to 201 of SEQ ID NO: 168, a G at position corresponding to 201 of SEQ ID NO: 169, a C at position corresponding to 201 of SEQ ID NO: 170, a T at position corresponding to 201 of SEQ ID NO: 171, a T at position corresponding to 201 of SEQ ID NO: 172, an A at position corresponding to 201 of SEQ ID NO: 173, an A at position corresponding to 201 of SEQ ID NO: 174, a T at position corresponding to 201 of SEQ ID NO: 175, a G at position corresponding to 201 of SEQ ID NO: 176, a C at position corresponding to 201 of SEQ ID NO: 177, a C at position corresponding to 201 of SEQ ID NO: 178, an A at position corresponding to 201 of SEQ ID NO: 179, an A at position corresponding to 201 of SEQ ID NO: 180, a G at position corresponding to 201 of SEQ ID NO: 181, a T at position corresponding to 201 of SEQ ID NO: 182, a C at position corresponding to 201 of SEQ ID NO: 183, an AG at position corresponding to 201 of SEQ ID NO: 184, a T at position corresponding to 201 of SEQ ID NO: 185, or a T at position corresponding to 201 of SEQ ID NO: 186; and performing a plant breeding step. The plant breeding step can be for example backcrossing the selected progeny plants to an elite yellow corn plant and further selecting further progeny plants for further traits selected from bn7, y1, at least three Ccd1 copies and any combination thereof, or for example, further selecting further progeny plants for further traits selected from bn7, y1, at least three Ccd1 copies, P1 and any combination thereof and producing doubled haploids from the further selected progeny plants, thereby producing a corn plant which comprises white kernels and otherwise comprises substantially all of the loci of an elite yellow corn plant.

In some embodiments, methods of producing a maize plant having white kernels are provided, by genotyping a nucleic acid isolated from a population of maize plants for the presence of a marker located within a chromosomal interval defined by and comprising SEQ ID NO: 148 and SEQ ID NO: 186 and comprising a C allele at a position corresponding to position 201 of SEQ ID NO: 157, selecting a plant on the basis of the presence of the genotyped marker, crossing the selected plant with a second plant to produce progeny seed, and selecting a progeny seed or plant grown therefrom comprising the C allele, to produce a plant having white kernels.

### BRIEF DESCRIPTION OF THE SEQUENCES

The sequence descriptions in Table 1 summarize the Sequence Listing attached hereto, which is hereby incorporated by reference. The Sequence Listing contains one letter codes for nucleotide sequence characters and single letter codes for amino acids as defined in the IUPAC-IUB standards described in Nucleic Acids Research 13:3021-3030 (1985) and in the Biochemical Journal 219 (2): 345-373 (1984).

TABLE 1
Sequence Listing Description

| SEQ | | | | | |
|---|---|---|---|---|---|
| ID | | | | Sequence | Sequence |
| NO: | Name | | Chromosome | Start | End |
| 1 | C104CVM-001-Q001 y1 | DNA | ZmChr6v2 | 85205509 | 85204757 |
| | indel MW yellow | | | | |
| 2 | C104CVM-001-X002 y1 | DNA | ZmChr6v2 | 85205242 | 85205229 |
| | “W” yellow probe | | | | |
| 3 | C104CVM-F002 y1 MW | DNA | ZmChr6v2 | 85205274 | 85205255 |
| | yellow primer | | | | |
| 4 | C104CVM-R002 y1 MW | DNA | ZmChr6v2 | 85205148 | 85205171 |
| | yellow primer | | | | |
| 5 | C104CVN-001-Q001 y1 | DNA | ZmChr6v2 | 85205509 | 85204757 |
| | indel MW white | | | | |
| 6 | C104CVN-001-X002 y1 | DNA | ZmChr6v2 | 85205367 | 85204963 |
| | “M” white probe | | | | |
| 7 | C104CNV-F002 y1 MW | DNA | ZmChr6v2 | 85205454 | 85205435 |
| | white primer | | | | |
| 8 | C104CVN-R002 y1 MW | DNA | ZmChr6v2 | 85204892 | 85204911 |
| | white primer | | | | |
| 9 | C104CVA-001-Q001 y1 | DNA | ZmChr6v2 | 85204471 | 85204871 |
| | SNP | | | | |
| 10 | C104CVA-001-X001 y1 | DNA | ZmChr6v2 | 85204665 | 85204676 |
| | yellow probe | | | | |
| 11 | C104CVA-001-X002 y1 | DNA | ZmChr6v2 | 85204665 | 85204676 |
| | white probe | | | | |
| 12 | C104CVA-F001 y1 primer | DNA | ZmChr6v2 | 85204586 | 85204605 |
| 13 | C104CVA-R001 y1 | DNA | ZmChr6v2 | 85204759 | 85204741 |
| | primer | | | | |
| 14 | C104CVB-001-Q001 y1 | DNA | ZmChr6v2 | 85204525 | 85204925 |
| | SNP | | | | |
| 15 | C104CVB-001-X001 y1 | DNA | ZmChr6v2 | 85204717 | 85204728 |
| | white probe | | | | |
| 16 | C104CVB-001-X002 y1 | DNA | ZmChr6v2 | 85204719 | 85204729 |
| | yellow probe | | | | |
| 17 | C104CVB-F001 y1 primer | DNA | ZmChr6v2 | 85204586 | 85204605 |
| 18 | C104CVB-R001 y1 | DNA | ZmChr6v2 | 85204759 | 85204741 |
| | primer | | | | |
| 19 | C104CWM-001-Q034 y1 | DNA | ZmChr6v2 | 85206445 | 85205867 |
| | SNP | | | | |
| 20 | C104CWM-001-X005 y1 | DNA | ZmChr6v2 | 85206153 | 85206135 |
| | yellow probe | | | | |
| 21 | C104CWM-001-X006 y1 | DNA | ZmChr6v2 | 85206152 | 85206137 |
| | white probe | | | | |
| 22 | C104CWM-F003 y1 | DNA | ZmChr6v2 | 85206200 | 85206181 |
| | primer | | | | |
| 23 | C104CWM-R002 y1 | DNA | ZmChr6v2 | 85206105 | 85206124 |
| | primer | | | | |
| 24 | C104CVP-001-Q001 y1 | DNA | ZmChr6v2 | 85202275 | 85201962 |
| | SNP7 | | | | |
| 25 | C104CVP-001-X001 y1 | DNA | ZmChr6v2 | 85202104 | 85202121 |
| | yellow probe | | | | |
| 26 | C104CVP-001-X002 y1 | DNA | ZmChr6v2 | 85202103 | 85202121 |
| | white probe | | | | |
| 27 | C104CVP-F001 y1 primer | DNA | ZmChr6v2 | 85202179 | 85202158 |
| 28 | C104CVP-R001 y1 | DNA | ZmChr6v2 | 85202082 | 85202101 |
| | primer | | | | |
| 29 | C104CY1-001-Q059 Wc1 | DNA | ZmChr9v2 | 157484784 | 157485221 |
| | P/X tam3ld_junction | | | | |
| 30 | C104CY1-001-X008 Wc1 | DNA | ZmChr9v2 | 157484993 | 157485014 |
| | yellow “X” probe | | | | |
| 31 | C104CY1-F003 Wc1 | DNA | ZmChr9v2 | 157484960 | 157484984 |
| | yellow primer | | | | |
| 32 | C104CY1-R001 Wc1 | DNA | ZmChr9v2 | 157485128 | 157485108 |
| | yellow primer | | | | |
| 33 | Endogenous adh1 | DNA | ZmChr1v2 | 286782027 | 286782009 |
| 34 | C00414-01-X003 Wc1 | DNA | ZmChr1v2 | 286782027 | 286782009 |
| | white “P” probe | | | | |
| 35 | C00414-F2 Wc1 white | DNA | ZmChr1v2 | 286782048 | 286782029 |
| | primer | | | | |
| 36 | C00414-R2 Wc1 white | DNA | ZmChr1v2 | 286781987 | 286782007 |
| | primer | | | | |
| 37 | Ccd1r qRT-PCR Ccd1- | DNA | ZmChr9v2 | 159698952 | 159698926 |
| | FAMTAMRA probe | | | | |
| | (Ccd1 copy number) | | | | |
| 38 | Ccd1r qRT-PCR Ccd1-F | DNA | ZmChr9v2 | 159698975 | 159698954 |
| | primer | | | | |
| 39 | Ccd1r qRT-PCR Ccd1-R | DNA | ZmChr9v2 | 159698900 | 159698923 |
| | primer | | | | |
| 40 | Ccd1r qRT-PCR C00414- | DNA | ZmChr1v2 | 286782027 | 286782009 |
| | 01-X003 probe | | | | |
| | (Internal control) | | | | |
| 41 | Ccd1r qRT-PCR C00414- | DNA | ZmChr1v2 | 286782048 | 286782029 |
| | F2 primer | | | | |
| 42 | Ccd1r qRT-PCR C00414- | DNA | ZmChr1v2 | 286781987 | 286782007 |
| | R2 primer | | | | |
| 43 | Zm00001d021505_T001 | DNA | ZmChr7v2 | 154511055 | 154517957 |
| | CDS | | | | |
| 44 | Zm00001d021506_T001 | DNA | ZmChr7v2 | 154660671 | 154661409 |
| | CDS | | | | |
| 45 | Zm00001d021507_T001 | DNA | ZmChr7v2 | 154664532 | 154669144 |
| | CDS | | | | |
| 46 | Zm00001d021508_T001 | DNA | ZmChr7v2 | 154712200 | 154818186 |
| | CDS | | | | |
| 47 | Zm00001d021509_T001 | DNA | ZmChr7v2 | 154810030 | 154810919 |
| | CDS | | | | |
| 48 | Zm00001d021510_T001 | DNA | ZmChr7v2 | 154812407 | 154813243 |
| | CDS | | | | |
| 49 | Zm00001d021511_T001 | DNA | ZmChr7v2 | 154712200 | 154818186 |
| | CDS | | | | |
| 50 | Zm00001d021512_T001 | DNA | ZmChr7v2 | 154839778 | 154845525 |
| | CDS | | | | |
| 51 | Zm00001d021513_T001 | DNA | ZmChr7v2 | 154982451 | 154984175 |
| | CDS | | | | |
| 52 | Zm00001d021514_T001 | DNA | ZmChr7v2 | 154891964 | 155006374 |
| | CDS | | | | |
| 53 | Zm00001d021515_T001 | DNA | ZmChr7v2 | 155011302 | 155012492 |
| | CDS | | | | |
| 54 | Zm00001d021516_T001 | DNA | ZmChr7v2 | 155050963 | 155068622 |
| | CDS | | | | |
| 55 | Zm00001d021517_T001 | DNA | ZmChr7v2 | 155221533 | 155241566 |
| | CDS | | | | |
| 56 | Zm00001d021518_T001 | DNA | ZmChr7v2 | 155224576 | 155230975 |
| | CDS | | | | |
| 57 | Zm00001d021519_T001 | DNA | ZmChr7v2 | 155201983 | 155322383 |
| | CDS | | | | |
| 58 | Zm00001d021520_T001 | DNA | ZmChr7v2 | 155287968 | 155290843 |
| | CDS | | | | |
| 59 | Zm00001d021521_T001 | DNA | ZmChr7v2 | 155350905 | 155351840 |
| | CDS | | | | |
| 60 | Zm00001d021522_T001 | DNA | ZmChr7v2 | 155354747 | 155358299 |
| | CDS | | | | |
| 61 | Zm00001d021524_T001 | DNA | ZmChr7v2 | 155402591 | 155411069 |
| | CDS | | | | |
| 62 | Zm00001d021525_T001 | DNA | ZmChr7v2 | 155412304 | 155413122 |
| | CDS | | | | |
| 63 | Zm00001d021526_T001 | DNA | ZmChr7v2 | 155414352 | 155415995 |
| | CDS | | | | |
| 64 | Zm00001d021528_T001 | DNA | ZmChr7v2 | 155436012 | 155442945 |
| | CDS | | | | |
| 65 | Zm00001d021529_T001 | DNA | ZmChr7v2 | 155444495 | 155444247 |
| | CDS | | | | |
| 66 | Zm00001d021532_T001 | DNA | ZmChr7v2 | 155506738 | 155510922 |
| | CDS | | | | |
| 67 | Zm00001d021533_T001 | DNA | ZmChr7v2 | 155518538 | 155519082 |
| | CDS | | | | |
| 68 | Zm00001d021534_T001 | DNA | ZmChr7v2 | 155520912 | 155524139 |
| | CDS | | | | |
| 69 | Zm00001d021535_T001 | DNA | ZmChr7v2 | 155537618 | 155541484 |
| | CDS | | | | |
| 70 | Zm00001d021536_T001 | DNA | ZmChr7v2 | 155545247 | 155545684 |
| | CDS | | | | |
| 71 | Zm00001d021537_T001 | DNA | ZmChr7v2 | 155585249 | 155586633 |
| | CDS | | | | |
| 72 | Zm00001d021538_T001 | DNA | ZmChr7v2 | 155651550 | 155652150 |
| | CDS | | | | |
| 73 | Zm00001d021540_T001 | DNA | ZmChr7v2 | 155657771 | 155659970 |
| | CDS | | | | |
| 74 | Zm00001d021541_T001 | DNA | ZmChr7v2 | 155662402 | 155676457 |
| | CDS | | | | |
| 75 | Zm00001d021542_T001 | DNA | ZmChr7v2 | 155706907 | 155707848 |
| | CDS | | | | |
| 76 | Zm00001d021544_T001 | DNA | ZmChr7v2 | 155773463 | 155772444 |
| | CDS | | | | |
| 77 | Zm00001d021545_T001 | DNA | ZmChr7v2 | 155776956 | 155779507 |
| | CDS | | | | |
| 78 | Zm00001d021546_T001 | DNA | ZmChr7v2 | 155791841 | 155797315 |
| | CDS | | | | |
| 79 | Zm00001d021551_T001 | DNA | ZmChr7v2 | 155919211 | 155922308 |
| | CDS | | | | |
| 80 | Zm00001d021552_T001 | DNA | ZmChr7v2 | 155957346 | 155961655 |
| | CDS | | | | |
| 81 | Zm00001d021553_T001 | DNA | ZmChr7v2 | 156002198 | 156011877 |
| | CDS | | | | |
| 82 | Zm00001d021554_T001 | DNA | ZmChr7v2 | 156043337 | 156051051 |
| | CDS | | | | |
| 83 | Zm00001d021557_T001 | DNA | ZmChr7v2 | 156161012 | 156162789 |
| | CDS | | | | |
| 84 | Zm00001d021558_T001 | DNA | ZmChr7v2 | 156229809 | 156251121 |
| | CDS | | | | |
| 85 | Zm00001d021562_T001 | DNA | ZmChr7v2 | 156344122 | 156345150 |
| | CDS | | | | |
| 86 | Zm00001d021564_T001 | DNA | ZmChr7v2 | 156395079 | 156395645 |
| | CDS | | | | |
| 87 | Zm00001d021565_T001 | DNA | ZmChr7v2 | 156395079 | 156414294 |
| | CDS | | | | |
| 88 | Zm00001d021566_T002 | DNA | ZmChr7v2 | 156429859 | 156432578 |
| | CDS | | | | |
| 89 | Zm00001d021567_T001 | DNA | ZmChr7v2 | 156494291 | 156532941 |
| | CDS | | | | |
| 90 | Zm00001d021568_T001 | DNA | ZmChr7v2 | 156447191 | 156448700 |
| | CDS | | | | |
| 91 | Zm00001d021569_T001 | DNA | ZmChr7v2 | 156436516 | 156513307 |
| | CDS | | | | |
| 92 | Zm00001d021570_T001 | DNA | ZmChr7v2 | 156800391 | 156803152 |
| | CDS | | | | |
| 93 | Zm00001d021571_T001 | DNA | ZmChr7v2 | 156823483 | 156824913 |
| | CDS | | | | |
| 94 | Zm00001d021572_T001 | DNA | ZmChr7v2 | 156617620 | 156828254 |
| | CDS | | | | |
| 95 | Zm00001d021573_T001 | DNA | ZmChr7v2 | 156833728 | 156850393 |
| | CDS | | | | |
| 96 | Zm00001d021574_T001 | DNA | ZmChr7v2 | 156873315 | 156875962 |
| | CDS | | | | |
| 97 | Zm00001d021576_T001 | DNA | ZmChr7v2 | 156613696 | 156618245 |
| | CDS | | | | |
| 98 | Zm00001d021577_T001 | DNA | ZmChr7v2 | 156613558 | 156828200 |
| | CDS | | | | |
| 99 | C105495-001-Q001 bn7 | DNA | ZmChr7v2 | 155543405 | 155543505 |
| 100 | C105495-001-X003 probe | DNA | ZmChr7v2 | 155543445 | 155543464 |
| | for SEQ ID NO: 99 WT = T | | | | |
| 101 | C105495-001-X004 probe | DNA | ZmChr7v2 | 155543443 | 155543464 |
| | for SEQ ID NO: 99, | | | | |
| | White = C | | | | |
| 102 | C105495-F001 primer for | DNA | ZmChr7v2 | 155543350 | 155543365 |
| | SEQ ID NO: 99 | | | | |
| 103 | C105495-R001 primer for | DNA | ZmChr7v2 | 155543479 | 155543460 |
| | SEQ ID NO: 99 | | | | |
| 104 | C10549P-001-Q001 bn7 | DNA | ZmChr7v2 | 156614153 | 156614253 |
| 105 | C10549P-001-X003 | DNA | ZmChr7v2 | 156614194 | 156614207 |
| | probe for SEQ ID NO: | | | | |
| | 104 WT = T | | | | |
| 106 | C10549P-001-X004 | DNA | ZmChr7v2 | 156614198 | 156614211 |
| | probe for SEQ ID NO: | | | | |
| | 104 White = C | | | | |
| 107 | C10549P-F001 primer for | DNA | ZmChr7v2 | 156614165 | 156614184 |
| | SEQ ID NO: 104 | | | | |
| 108 | C10549P-R001 primer for | DNA | ZmChr7v2 | 156614259 | 156614240 |
| | SEQ ID NO: 104 | | | | |
| 109 | cDNA encoding Y1 | DNA | | — | — |
| 110 | Y1 polypeptide | PRT | | — | — |
| 111 | cDNA encoding Ccd | DNA | | — | — |
| 112 | Ccd1 polypeptide | PRT | | — | — |
| 113 | cDNA encoding cob color | DNA | | — | — |
| | P1 | | | | |
| 114 | cob color P1 polypeptide | PRT | | — | — |
| 115 | C10591M-001-K001 | DNA | ZmChr7v2 | 147342214 | 115 |
| 116 | C10591N-001-K001 | DNA | ZmChr7v2 | 147599495 | 115.48 |
| 117 | C10591P-001-K001 | DNA | ZmChr7v2 | 147881425 | 116 |
| 118 | C10591R-001-K001 | DNA | ZmChr7v2 | 148349658 | 116.87 |
| 119 | C10591T-001-K001 | DNA | ZmChr7v2 | 148445714 | 117.05 |
| 120 | Zmchr7v2_148557381 | DNA | ZmChr7v2 | 148557381 | 117.26 |
| 121 | C10591U-001-K001 | DNA | ZmChr7v2 | 148647378 | 117.43 |
| 122 | C10591V-001-K001 | DNA | ZmChr7v2 | 149251029 | 118.12 |
| 123 | C10591W-001-K001 | DNA | ZmChr7v2 | 150205260 | 118.42 |
| 124 | C10591X-001-K001 | DNA | ZmChr7v2 | 151245301 | 119.03 |
| 125 | C10591Y-001-K001 | DNA | ZmChr7v2 | 151944703 | 119.44 |
| 126 | C105920-001-K001 | DNA | ZmChr7v2 | 152918240 | 120.01 |
| 127 | C105921-001-K001 | DNA | ZmChr7v2 | 153902127 | 120.57 |
| 128 | ZmChr7v2_153902319 | DNA | ZmChr7v2 | 153902319 | 120.57 |
| 129 | ZmChr7v2_154022588 | DNA | ZmChr7v2 | 154022588 | 120.59 |
| 130 | ZmChr7v2_154156304 | DNA | ZmChr7v2 | 154156304 | 120.6 |
| 131 | ZmChr7v2_154330403 | DNA | ZmChr7v2 | 154330403 | 120.62 |
| 132 | ZmChr7v2_154330549 | DNA | ZmChr7v2 | 154330549 | 120.62 |
| 133 | ZmChr7v2_154330550 | DNA | ZmChr7v2 | 154330550 | 120.62 |
| 134 | ZmChr7v2_154330751 | DNA | ZmChr7v2 | 154330751 | 120.62 |
| 135 | ZmChr7v2_154331150 | DNA | ZmChr7v2 | 154331150 | 120.62 |
| 136 | ZmChr7v2_154331279 | DNA | ZmChr7v2 | 154331279 | 120.62 |
| 137 | ZmChr7v2_154331450 | DNA | ZmChr7v2 | 154331450 | 120.62 |
| 138 | ZmChr7v2_155005881 | DNA | ZmChr7v2 | 155005881 | 120.7 |
| 139 | ZmChr7v2_155012364 | DNA | ZmChr7v2 | 155012364 | 120.7 |
| 140 | ZmChr7v2_155053571 | DNA | ZmChr7v2 | 155053571 | 120.71 |
| 141 | ZmChr7v2_155241237 | DNA | ZmChr7v2 | 155241237 | 120.73 |
| 142 | ZmChr7v2_155241535 | DNA | ZmChr7v2 | 155241535 | 120.73 |
| 143 | ZmChr7v2_155288963 | DNA | ZmChr7v2 | 155288963 | 120.73 |
| 144 | ZmChr7v2_155289435 | DNA | ZmChr7v2 | 155289435 | 120.73 |
| 145 | ZmChr7v2_155351605 | DNA | ZmChr7v2 | 155351605 | 120.74 |
| 146 | ZmChr7v2_155410715 | DNA | ZmChr7v2 | 155410715 | 120.89 |
| 147 | ZmChr7v2_155413697 | DNA | ZmChr7v2 | 155413697 | 120.89 |
| 148 | ZmChr7v2_155442292 | DNA | ZmChr7v2 | 155442292 | 120.97 |
| 149 | C105922-001-K001 | DNA | ZmChr7v2 | 155492309 | 121.09 |
| 150 | ZmChr7v2_155506719 | DNA | ZmChr7v2 | 155506719 | 121.12 |
| 151 | ZmChr7v2_155508499 | DNA | ZmChr7v2 | 155508499 | 121.13 |
| 152 | ZmChr7v2_155518701 | DNA | ZmChr7v2 | 155518701 | 121.15 |
| 153 | ZmChr7v2_155518918 | DNA | ZmChr7v2 | 155518918 | 121.15 |
| 154 | ZmChr7v2_155519232 | DNA | ZmChr7v2 | 155519232 | 121.15 |
| 155 | ZmChr7v2_155519405 | DNA | ZmChr7v2 | 155519405 | 121.16 |
| 156 | ZmChr7v2_155520657 | DNA | ZmChr7v2 | 155520657 | 121.16 |
| 157 | ZmChr7v2_155521495 | DNA | ZmChr7v2 | 155521495 | 121.16 |
| 158 | ZmChr7v2_155521516 | DNA | ZmChr7v2 | 155521516 | 121.16 |
| 159 | ZmChr7v2_155521591 | DNA | ZmChr7v2 | 155521591 | 121.16 |
| 160 | ZmChr7v2_155524172 | DNA | ZmChr7v2 | 155524172 | 121.17 |
| 161 | ZmChr7v2_155539594 | DNA | ZmChr7v2 | 155539594 | 121.2 |
| 162 | ZmChr7v2_155540225 | DNA | ZmChr7v2 | 155540225 | 121.21 |
| 163 | ZmChr7v2_155586401 | DNA | ZmChr7v2 | 155586401 | 121.32 |
| 164 | ZmChr7v2_155663891 | DNA | ZmChr7v2 | 155663891 | 121.34 |
| 165 | ZmChr7v2_155666082 | DNA | ZmChr7v2 | 155666082 | 121.34 |
| 166 | ZmChr7v2_155675886 | DNA | ZmChr7v2 | 155675886 | 121.34 |
| 167 | ZmChr7v2_155675939 | DNA | ZmChr7v2 | 155675939 | 121.34 |
| 168 | ZmChr7v2_155676116 | DNA | ZmChr7v2 | 155676116 | 121.34 |
| 169 | ZmChr7v2_155676268 | DNA | ZmChr7v2 | 155676268 | 121.34 |
| 170 | ZmChr7v2_155680489 | DNA | ZmChr7v2 | 155680489 | 121.34 |
| 171 | ZmChr7v2_155768407 | DNA | ZmChr7v2 | 155768407 | 121.37 |
| 172 | C105923-001-K001 | DNA | ZmChr7v2 | 156254131 | 121.49 |
| 173 | ZmChr7v2_156404407 | DNA | ZmChr7v2 | 156404407 | 121.53 |
| 174 | ZmChr7v2_156404423 | DNA | ZmChr7v2 | 156404423 | 121.53 |
| 175 | ZmChr7v2_156413328 | DNA | ZmChr7v2 | 156413328 | 121.53 |
| 176 | ZmChr7v2_156413350 | DNA | ZmChr7v2 | 156413350 | 121.53 |
| 177 | ZmChr7v2_156413461 | DNA | ZmChr7v2 | 156413461 | 121.53 |
| 178 | ZmChr7v2_156413676 | DNA | ZmChr7v2 | 156413676 | 121.53 |
| 179 | ZmChr7v2_156413913 | DNA | ZmChr7v2 | 156413913 | 121.53 |
| 180 | ZmChr7v2_156413983 | DNA | ZmChr7v2 | 156413983 | 121.53 |
| 181 | ZmChr7v2_156414056 | DNA | ZmChr7v2 | 156414056 | 121.53 |
| 182 | ZmChr7v2_156414135 | DNA | ZmChr7v2 | 156414135 | 121.53 |
| 183 | ZmChr7v2_156414147 | DNA | ZmChr7v2 | 156414147 | 121.53 |
| 184 | ZmChr7v2_156415059 | DNA | ZmChr7v2 | 156415059 | 121.53 |
| 185 | ZmChr7v2_156612669 | DNA | ZmChr7v2 | 156612669 | 121.59 |
| 186 | ZmChr7v2_156613548 | DNA | ZmChr7v2 | 156613548 | 121.59 |
| 187 | ZmChr7v2_156617084 | DNA | ZmChr7v2 | 156617084 | 121.59 |
| 188 | ZmChr7v2_157041859 | DNA | ZmChr7v2 | 157041859 | 121.7 |
| 189 | ZmChr7v2_157056914 | DNA | ZmChr7v2 | 157056914 | 121.7 |
| 190 | ZmChr7v2_157057145 | DNA | ZmChr7v2 | 157057145 | 121.7 |
| 191 | ZmChr7v2_157146769 | DNA | ZmChr7v2 | 157146769 | 121.73 |
| 192 | ZmChr7v2_157162320 | DNA | ZmChr7v2 | 157162320 | 121.74 |
| 193 | ZmChr7v2_157162696 | DNA | ZmChr7v2 | 157162696 | 121.74 |
| 194 | ZmChr7v2_157163045 | DNA | ZmChr7v2 | 157163045 | 121.74 |
| 195 | ZmChr7v2_157384566 | DNA | ZmChr7v2 | 157384566 | 121.82 |
| 196 | ZmChr7v2_157385181 | DNA | ZmChr7v2 | 157385181 | 121.82 |
| 197 | ZmChr7v2_157385619 | DNA | ZmChr7v2 | 157385619 | 121.82 |
| 198 | ZmChr7v2_157386399 | DNA | ZmChr7v2 | 157386399 | 121.82 |
| 199 | ZmChr7v2_157547698 | DNA | ZmChr7v2 | 157547698 | 121.88 |
| 200 | ZmChr7v2_157547871 | DNA | ZmChr7v2 | 157547871 | 121.88 |
| 201 | ZmChr7v2_157547933 | DNA | ZmChr7v2 | 157547933 | 121.88 |
| 202 | ZmChr7v2_157622874 | DNA | ZmChr7v2 | 157622874 | 121.91 |
| 203 | Zmchr7v2_157833774 | DNA | ZmChr7v2 | 157833774 | 121.99 |
| 204 | C105924-001-K001 | DNA | ZmChr7v2 | 157910690 | 122.01 |
| 205 | C105925-001-K001 | DNA | ZmChr7v2 | 158344641 | 122.72 |
| 206 | C105926-001-K001 | DNA | ZmChr7v2 | 160759360 | 126.82 |
| 207 | C105927-001-K001 | DNA | ZmChr7v2 | 162349934 | 128.79 |
| 208 | C105928-001-K001 | DNA | ZmChr7v2 | 163568839 | 130.41 |
| 209 | C0031KD-001 | DNA | | | |

TABLE 1a
Allele Calls for Markers

| SEQ | | | |
|---|---|---|---|
| ID NO: | Designation | Position | Meaning |
| 5 | C104CVN-001-Q001 y1 | indel | Deletion (white) cf. |
| | | | SEQ ID NO: 1 (yellow) |
| 9 | C104CVA-001-Q001 y1 | 201 | A = yellow; G = white |
| 14 | C104CVB-001-Q001 y1 | 201 | T = white; C = yellow |
| 19 | C104CWM-001-Q034 y1 | 301 | T = yellow; C = white |
| 24 | C104CVP-001-Q001 y1 | 158 | A = yellow; C = white |
| 99 | C105495-001-Q001 bn7 | 51 | C = white; T = yellow/WT |
| 104 | C10549P-001-Q001 bn7 | 51 | C = white; T = yellow/WT |

### DETAILED DESCRIPTION

Maize (Zea mays) can be referred to as maize or corn. Disclosed are methods of producing or using non-yellow or white corn-kernelled plants and seeds and the plants, seeds, plant parts and compositions relating to or resulting from such methods. In some embodiments, elite yellow germplasm is used to generate improved white corn varieties. As an example, yellow corn lines are crossed with white corn lines to recover elite yellow genetics that contain and are fixed for desirable alleles that produce white corn color. Certain definitions used in the specification are provided below. In some embodiments, white corn germplasm is more efficiently identified and selected and used to cross with other corn germplasm.

ALLELE: Any of one or more alternative forms of a genetic sequence.

In a diploid cell or organism, the two alleles of a given sequence typically occupy corresponding loci on a pair of homologous chromosomes.

BACKCROSSING: Process in which a breeder crosses a hybrid progeny variety back to one of the parental genotypes one or more times.

BREEDING CROSS: A cross to introduce new genetic material into a plant for the development of a new variety. For example, one could cross plant A with plant B, wherein plant B would be genetically different from plant A. After the breeding cross, the resulting F1 plants could then be selfed or sibbed for one, two, three or more times (F1, F2, F3, etc.) until a new inbred variety is developed.

CELL: Cell as used herein includes a plant cell, whether isolated, in tissue culture or incorporated in a plant or plant part.

CROSS POLLINATION: Fertilization by the union of two gametes from different plants.

CROSSING: The combination of genetic material by traditional methods such as a breeding cross or backcross, but also including protoplast fusion and other molecular biology methods of combining genetic material from two sources.

F1 PROGENY: A progeny plant produced by crossing a plant of one maize line with a plant of another maize line.

HYBRID VARIETY: A substantially heterozygous hybrid line and minor genetic modifications thereof that retain the overall genetics of the hybrid line.

INBRED: A variety developed through inbreeding or doubled haploidy that preferably comprises homozygous alleles at about 95% or more of its loci. An inbred can be reproduced by selfing or growing in isolation so that the plants can only pollinate with the same inbred variety.

INTROGRESSION: The process of transferring genetic material from one genotype to another.

LINKAGE: Refers to a phenomenon wherein alleles on the same chromosome tend to segregate together more often than expected by chance if their transmission was independent.

LOCUS: A specific location on a chromosome.

LOCUS CONVERSION (Also called TRAIT CONVERSION): A locus conversion refers to plants within a variety that have been modified in a manner that retains the overall genetics of the variety and further comprises one or more loci with a specific desired trait, such as kernel color, cob color, male sterility, insect resistance, disease resistance or herbicide tolerance or resistance. Examples of single locus conversions include mutant genes, transgenes and native traits finely mapped to a single locus. One or more locus conversion traits may be introduced into a single corn variety.

NON-YELLOW describes a corn plant, seed, kernel or plant part that is homozygous or heterozygous for an allele that is associated with and contributes to a white or whiter kernel, seed or seed part or that has sufficient copy number of a gene to contribute to a white or whiter kernel, seed or seed part and includes white kernels and seeds. If the allele associated with a white or whiter seed, seed part or kernel is a recessive allele or co-dominant allele a non-yellow seed, seed part or kernel may still have a yellow or other non-white color.

PERCENT IDENTITY: Percent identity as used herein with respect to plant genetics refers to the comparison of the alleles present in two plant varieties. For example, when comparing two inbred plants to each other, each inbred plant will have the same allele (and therefore be homozygous) at almost all of their loci. Percent identity is determined by comparing a statistically significant number of the homozygous alleles of two varieties.

PLANT: As used herein, the term “plant” includes reference to an immature or mature whole plant, including a plant that has been detasseled or from which seed or grain has been removed. Seed or embryo that will produce the plant is also considered to be the plant.

PLANT PART: As used herein, the term “plant part” includes leaves, stems, roots, seed, grain, embryo, pollen, ovules, flowers, ears, cobs, husks, stalks, root tips, anthers, pericarp, silk, tissue, cells and the like.

RESISTANCE: Synonymous with tolerance. The ability of a plant to withstand exposure to an insect, disease, herbicide or other condition. A resistant plant variety will have a level of resistance higher than a comparable wild-type variety.

SEED: Fertilized and ripened ovule, consisting of the plant embryo, stored food material, and a protective outer seed coat. Synonymous with grain.

SEED PART includes any part of a seed, including the endosperm, embryo, seed coat, pericarp and testa, and aleurone.

SELF POLLINATION: A plant is self-pollinated if pollen from one flower is transferred to the same or another flower of the same plant.

SNP: SINGLE-NUCLEOTIDE POLYMORPHISM: is a DNA sequence variation occurring when a single nucleotide in the genome differs between individual plant or plant varieties. The differences can be equated with different alleles and indicate polymorphisms. A number of SNP markers can be used to determine a molecular profile of an individual plant or plant variety and can be used to compare similarities and differences among plants and plant varieties.

SSRs: Genetic markers based on polymorphisms in repeated nucleotide sequences, such as microsatellites. A marker system based on SSRs can be informative in linkage analysis relative to other marker systems in that multiple alleles may be present.

VARIETY: A maize line and minor genetic modifications thereof that retain the overall genetics of the line including but not limited to a locus conversion, a mutation, or a somoclonal variant.

As used herein, the phrase “elite line” refers to any line that is substantially homozygous, or a hybrid variety derived from the cross of two substantially homozygous parents and has resulted from breeding and selection for superior agronomic performance. When the terms yellow corn line, yellow line, non-yellow corn line, non-yellow line, white corn line and white line are used herein, those terms are intended to include an elite yellow corn line, elite yellow line, elite white corn line or elite white line as appropriate.

Kernel color in maize is affected by the rate of production and degradation of various pigments including carotenoids, which contribute a yellow color. In the absence of carotenoids, the color and whiteness of kernels can be affected by the expression and accumulation of pigments in the aleurone and pericarp layers. Provided are a number of loci and related markers which are associated with white corn color which can be used in the methods described herein to produce plants having white or whiter kernels. Such loci include Yellow Endosperm 1 (Y1; SEQ ID NO: 109 encoding SEQ ID NO: 110), White cap (Wc1 comprising in white corn multiple copies of Ccd1; SEQ ID NO: 111 encoding SEQ ID NO: 112), Brown Aleurone which is contributed to by the Bn1 locus (located at about genetic position 130 CM and 150 cM on chromosome 7) and Bn7 disclosed herein, as well as P1 which relates to white pericarp and cob color (P1 cob color SEQ ID NO: 113 encoding SEQ ID NO: 114), which are desirable in white corn varieties. Y1 encodes phytoene synthase 1 (PSY1), an enzyme in the carotenoid biosynthesis pathway. Dominant alleles of the Y1 gene (Y1) confer expression of a yellow-grain phenotype, whereas recessive y1 alleles produce a white-grain phenotype. Dominant alleles for brown aleurone have been found to cause a brown color in white endosperm grain, whereas recessive brown aleurone alleles have been shown herein to be desirable for white corn. Dominant alleles for the Bn7 locus (designated Bn7) have been found to cause a lemon color in white endosperm grain and a brown pigment color in the aleurone visible under a microscope, whereas recessive alleles (designated bn7) have been shown herein to be desirable for white corn. Disclosed herein are Bn7 alleles which in homozygous or heterozygous state in a recessive y1/y1 background cause a lemon-yellow color, whereas the double recessive bn7/bn7 in a y1/y1 background result in whiter corn. In a Y1/y1 or Y1/Y1 background, the effect of the presence or absence of Bn7 on kernel color is masked by the yellow phenotype from the Y1 allele. The Wc locus contains genes encoding carotenoid cleavage dioxygenase 1 (Ccd1) which cleaves various carotenoids; the Wc locus in corn contains one or multiple tandem copies of the Ccd1 gene. Dominant alleles of Wc confer a white-endosperm phenotype (designated Wc alleles or multiple copies of Ccd1). Copy number variability of Ccd1 causes differing degrees of degradation in beta carotene content in endosperm, with more copies resulting in whiter endosperm. In a Bn7 y1 background, Wc alleles (multiple copies of Ccd1) inhibit accumulation of the brown pigment and produce a more intense white-endosperm phenotype. Increased copy number of the Ccd1 gene (Wc alleles) will improve whiteness in many genetic backgrounds, including y1/y1 with bn7/bn7 and other allele combinations, with stronger effects on improved whiteness observed as the copy number of Ccd1 increases. At least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or at least 30, at least 40, at least 50, at least 60, at least 70, at least 80, at least 90 or at least 100 copies of Ccd1 may be present.

Additionally, Pericarp color 1 (P1) which regulates red pigment in cob and pericarp can be used. Pericarp color has the allele nomenclature P1-pericarp color/cob color, such as P1-wr (P1-white pericarp/red cob), and P1-ww (P1-white pericarp/white cob), with recessive P1-ww desirable for white kernels and in white corn lines, including inbreds and hybrids. In some embodiments, the populations which can be used are fixed for white pericarp. For example, elite maize lines may be fixed for P1 white pericarp (P1-w_) and can be selected based on red/white cob.

In some embodiments, y1, Wc1, bn7, P1, Bn7 their associated markers and any combination thereof are used in plant breeding to select for white corn plants or plants expressing desirable alleles for white or non-yellow corn. The methods disclosed herein allow for increased selection intensity for other agronomic traits while selecting for white or non-yellow kernels, such as in the same marker assay or breeding stage or step, and permit improvements in genetic gain from a yellow parent, such as an elite yellow parent. One or more of a white corn line, a non-yellow corn line or yellow corn line may be included in the initial cross to generate a population. Populations comprising genetics which result from a yellow corn breeding program and populations which have or carry non-yellow traits can be used. Methods include introgressing or selecting for desirable alleles for or non-yellow, white or whiter kernels into or in the yellow-colored kernel parent genetic background or genome or from a population of plants that are heterozygous for one or more of the loci. In some embodiments, a progeny plant is recovered using the methods disclosed herein that has white kernels, optionally a white cob, contains at least one, at least 2, at least 3, or at least 4 of the loci described herein which contribute to a white corn phenotype and has at least 75%, 80%, 85%, 90%, 95%, 96%, 97%, 98% or 99% of the genetics or loci by pedigree, or all or substantially all of the loci of the elite yellow colored kernel parent, such as might be achieved through backcross introgression of alleles desirable for white corn from a white corn line parent into a yellow elite corn line parent. Also provided are F1 hybrids which have an inbred parent that has at least 75%, 80%, 85%, 90%, 95%, 96%, 97%, 98% or 99% of the genetics or loci of the elite yellow colored kernel parent by pedigree. In some embodiments, a progeny plant is recovered using the methods disclosed herein that has white or non-yellow kernels, optionally a white cob, and contains at least one, at least 2, at least 3, or at least 4 of the loci described herein which contribute to a white seed or kernel phenotype and/or are non-yellow. The progeny plant can be an inbred plant produced through double haploid or pedigree selection plant breeding or an F1 hybrid produced by crossing inbred parents. The methods disclosed herein provide increased efficiency in plant breeding and population development as fewer individuals can be screened to ensure recovery of a plurality of loci relating to white corn, such as at least one, at least two at least three, at least four or at least five loci, and less than ten, less than nine, less than eight, less than seven, less than six or less than five loci. In some embodiments, cob color is selected for P1-ww. With a fully functional set of markers and assays, the loci contributing to the white corn phenotype can be selected for or introgressed with a targeted recovery of genetics the elite yellow corn line.

Bn7 markers disclosed herein, and which can be used in any of the methods described herein include those listed in SEQ ID NOs: 43-99, 104 and 115-208 and those that are within 0.1 cM, 0.2 CM, 0.5 CM, 1 CM, 2 CM, 3 CM, 4 CM, 5 CM or 10 cM of any of SEQ ID NO: 43-99, 104 and 115-208. Bn7 markers that fall between and optionally include any two markers selected from SEQ ID NOs: 99, 104 and 115-208 are also disclosed and provided. For example, a region containing markers useful in the methods disclosed herein may begin with and include a sequence selected from SEQ ID NOs 99, 104 and 115-207 and end with and include a sequence selected from SEQ ID NOs 99, 104 and 116-208. Sequences that can be used in the methods and compositions disclosed herein may begin with position 1, 10, 20, 30, 40, 50, 60, 70, 80, 90, 100, 110, 120, 130, 140, 150, 160, 170, 180 or 190 and end at any one of positions 400, 390, 380, 370, 360, 350, 340, 330, 320, 310, 300, 290, 280, 270, 260, 250, 240, 230, 220 or 210 for any one of SEQ ID NOs 115-208. Markers in the reverse complement strand of sequences provided herein are also envisaged and can be used in the methods and compositions described herein. The methods disclosed herein for detection of Bn7 can be used with any one or more of markers for SEQ ID NO: 43-99, 104 and 115-208, such as to detect and select for white or non-yellow corn plants or seeds in plant breeding including the steps of crossing and/or selfing plants such as to generate segregating populations and the separation of white or non-yellow seeds or plants from plants carrying Bn7 alleles which do not contribute to whiteness of the kernel. Plants identified, selected, separated or grown in the methods disclosed herein can include one or more of the white or yellow Bn7 allele calls described in Example 3 and shown in Table 4.

In some embodiments, the corn plant used or produced in the methods disclosed herein such as identified, selected, separated or used to generate a cross or used in breeding or for further selection comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25 or at least 30 or more of the following: a C at position corresponding to 51 of SEQ ID NO: 99, a C at position corresponding to 51 of SEQ ID NO: 104, a G at position corresponding to 201 of SEQ ID NO: 148, a G at position corresponding to 201 of SEQ ID NO: 149, a C at position corresponding to 201 of SEQ ID NO: 150, a C at position corresponding to 201 of SEQ ID NO: 151, a C at position corresponding to 201 of SEQ ID NO: 152, a T at position corresponding to 201 of SEQ ID NO: 153, a G at position corresponding to 201 of SEQ ID NO: 154, a G at position corresponding to 201 of SEQ ID NO: 155, a T at position corresponding to 201 of SEQ ID NO: 156, a C at position corresponding to 201 of SEQ ID NO: 157, a G at position corresponding to 201 of SEQ ID NO: 158, a C at position corresponding to 201 of SEQ ID NO: 159, a G at position corresponding to 201 of SEQ ID NO: 160, a G at position corresponding to 201 of SEQ ID NO: 161, a G at position corresponding to 201 of SEQ ID NO: 162, an A at position corresponding to 201 of SEQ ID NO: 163, an A at position corresponding to 201 of SEQ ID NO: 164, a G at position corresponding to 201 of SEQ ID NO: 165, a T at position corresponding to 201 of SEQ ID NO: 166, a C at position corresponding to 201 of SEQ ID NO: 167, a G at position corresponding to 201 of SEQ ID NO: 168, a G at position corresponding to 201 of SEQ ID NO: 169, a C at position corresponding to 201 of SEQ ID NO: 170, a T at position corresponding to 201 of SEQ ID NO: 171, a T at position corresponding to 201 of SEQ ID NO: 172, an A at position corresponding to 201 of SEQ ID NO: 173, an A at position corresponding to 201 of SEQ ID NO: 174, a T at position corresponding to 201 of SEQ ID NO: 175, a G at position corresponding to 201 of SEQ ID NO: 176, a C at position corresponding to 201 of SEQ ID NO: 177, a C at position corresponding to 201 of SEQ ID NO: 178, an A at position corresponding to 201 of SEQ ID NO: 179, an A at position corresponding to 201 of SEQ ID NO: 180, a G at position corresponding to 201 of SEQ ID NO: 181, a T at position corresponding to 201 of SEQ ID NO: 182, a C at position corresponding to 201 of SEQ ID NO: 183, an AG at position corresponding to 201 of SEQ ID NO: 184, a T at position corresponding to 201 of SEQ ID NO: 185, or a T at position corresponding to 201 of SEQ ID NO: 186, or any combination thereof. The corn plant may comprise in combination with one or more of the forementioned the white allele for other SNPs listed in Table 4.

In some embodiments, substantially similar nucleic acid sequences encompassed by this disclosure are those sequences that are at least about or about 80% identical to the nucleic acid fragments reported herein or which are at least about or about 80% identical to any portion of the nucleotide sequences reported herein. Nucleic acid fragments which are at least 90% or at least 95% identical to the nucleic acid sequences reported herein, or which are at least 90% or at least 95% identical to any portion of the nucleotide sequences reported herein are also provided. It is well understood by one skilled in the art that many levels of sequence identity are useful in identifying related polynucleotide sequences. Useful examples of percent identities are those listed above, or also preferred is any integer percentage from 70% to 100%, such as at least, at least about or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In some embodiments, the polypeptide sequences or isolated or modified sequences disclosed herein comprise a polypeptide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity of SEQ ID NOS: 2, 4, 6,8 10, 12, 1,4 16, 18, 20, 22, 24, 27 or 28.

Provided are substantially similar sequences useful in compositions and methods provided herein. A “substantially similar sequence” generally refers to variants of the disclosed sequences such as those that result from site-directed mutagenesis, as well as synthetically derived sequences. A substantially similar promoter sequence of the present disclosure also generally refers to those fragments of a particular promoter nucleotide sequence disclosed herein that operate to promote the constitutive expression of an operably linked heterologous nucleic acid fragment. These promoter fragments comprise at least about 20 contiguous nucleotides, at least about 50 contiguous nucleotides, at least about 75 contiguous nucleotides, at least about 100 contiguous nucleotides of the particular promoter nucleotide sequence disclosed herein or a sequence that is at least 95 to about 99% identical to such contiguous sequences. The nucleotides of such fragments will usually include the TATA recognition sequence (or CAAT box or a CCAAT) of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. Variants of these promoter fragments, such as those resulting from site-directed mutagenesis, are encompassed by the compositions of the present disclosure.

Provided are sequences which contain one or more degenerate codons to those provided in the sequence listing. “Codon degeneracy” generally refers to divergence in the genetic code permitting variation of the nucleotide sequence without affecting the amino acid sequence of an encoded polypeptide. Accordingly, the instant disclosure relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent identity calculations may be determined using a variety of comparison methods designed to detect similar or identical sequences including, but not limited to, the MEGALIGN® program of the LASERGENER bioinformatics computing suite (DNASTAR® Inc., Madison, WI). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v 6.1 program of the LASERGENER bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In some embodiments, the % sequence identity is determined over the entire length of the molecule (nucleotide or amino acid). A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN generally refers to a BLAST program that compares a nucleotide query sequence against a nucleotide sequence database.

Methods for improving, increasing or enhancing the whiteness of a kernel can include modifying one or more of SEQ ID NO: 43-98 or the polypeptide encoded by these sequences to create locus conversions such as using transformation or genome editing techniques such as described herein. Any one or more of SEQ ID NO: 43-98 or an operably linked sequence can be modified to reduce or increase the expression, amount or activity of the polypeptide encoded by the sequence. For example, operably linked regulatory sequences such as promotor regions may be modified to increase or decrease expression and/or the coding sequence may be disrupted or modified such as through one or more SNPS, insertions or deletions, or a combination thereof. Provided are methods for making such plants, and the modified plants and seeds produced.

Also provided are hybrid plants which contain 50% or 25% of their genetics from an inbred corn line produced using the methods described herein and methods for makes such hybrids by conducting a cross with a different inbred line, and optionally producing a three-way or four-way (double cross) hybrid by crossing the F1 hybrid with another inbred line or F1 hybrid respectively. The parents can be one or more non-yellow or white corn lines. The hybrid can be selfed to produce hybrid grain or crossed with another plant in a plant breeding program. F1 hybrid plants and seeds and F2 hybrid plants, seed and grain are provided.

“Contributing to” or “contribute to” white or whiter kernels, seed or seed parts with respect to the genes, alleles and loci disclosed or envisaged herein means that the presence of the allele, gene or locus as favorable for a white or whiter seed, seed part or kernel phenotype in one or more genetic backgrounds. For example, in the presence of certain dominant alleles, contributions to whiteness such as from other loci may be masked, but their presence would still be considered as contributing to white or whiter kernels as removal of the masking allele facilitates observance of their contribution. In cases where whiteness is from a recessive locus, both alleles may need to be present to result in a white or whiter phenotype, but the presence of a single recessive allele in a heterozygous state would still be considered as contributing to white or whiter kernels. In the context of a plant breeding program, it may be desirable to hold any of the loci contributing to white kernels or seeds in heterozygous form, such that other elite alleles that originate with alleles relating to a yellow seed or kernel phenotype are preserved in the progeny and can be selected with the non-yellow alleles using subsequent plant breeding steps.

Populations or breeding pools can be generated by crossing lines carrying different alleles at some or many loci. For example, by crossing an elite yellow corn line with one or more lines carrying alleles favorable for white corn (e.g., non-yellow alleles) or encoding sequences that create a white corn phenotype such as disclosed herein, a population of plants homozygous and heterozygous at various loci can be generated. In some embodiments, such as by crossing a yellow corn line with a white corn line, generating segregating or bulk populations and conducting backcrossing to a parent line, from which populations samples can be taken, such as by seed chipping, the use of the methods disclosed herein reduces the number of plants needed to be planted to get sufficient plants having the desired state (homozygous for the desired trait or heterozygous carrying at least one desired copy of the desired trait). As an example, samples selected from and assayed from bulk populations generated during plant breeding can be found at ratios of 1 in 64 instead of 1 in 4000 or more. Ratios of plants carrying at least 1, at least 2, at least 3, at least 4 or at least 5 alleles and less than 10, less than 9, less than 8, less than 7, less than 6, less than 5, less than 4 or less than 3 alleles (such as Y1, Ccd1, P1, Bn1, Bn7) in the populations that the methods facilitate include ratios of less than 1 in 512, less than 1 in 256, less than 1 in 128, less than 1 in 64, less than 1 in 32 or less than 1 in 16. The number of lines needed in a bulk population to identify plants from which white corn lines comprising the desirable alleles are selected can be at least 10, at least 15, at least 20, at least 30, at least 40, at least 50, or at least 100 lines and less than 2500, less than 2000, less than 1500, less than 1000, less than 500, less than 250, less than 200, less than 150, less than 100, less than 90, or less than 80 lines. By contrast, in the absence of the methods, markers and loci described herein, much larger populations would need to be screened to have a sufficiently high probability of recovering a plant comprising each of the alleles that contribute to or are desirable for a white corn phenotype.

Any suitable methods of isolating nucleic acids from maize plants and for performing genetic marker profiles using SNP and SSR polymorphisms can be used with the markers, loci and methods described herein. SNPs are genetic markers based on a polymorphism in a single nucleotide. A marker system based on SNPs can be highly informative in linkage analysis relative to other marker systems in that multiple alleles may be present.

The methods disclosed herein can include a step of isolating nucleic acids, such as DNA, from a plant, a plant part, plant cell or a seed. The methods can include mechanical, electrical and/or chemical disruption of the plant, plant part, plant cell or seed, contacting the disrupted plant, plant part, plant cell or seed with a buffer or solvent, to produce a solution or suspension comprising nucleic acids, optionally contacting the nucleic acids with a precipitating agent to precipitate the nucleic acids, optionally extracting the nucleic acids, and optionally separating the nucleic acids such as by centrifugation or by binding to beads or a column, with subsequent elution, or a combination thereof. If DNA is being isolated, an RNase can be included in one or more of the method steps. The nucleic acids isolated can comprise all or substantially all of the genomic DNA sequence, all or substantially all of the chromosomal DNA sequence or all or substantially all of the coding sequences (cDNA) of the plant, plant part, or plant cell from which they were isolated. The amount and type of nucleic acids isolated may be sufficient to permit whole genome sequencing of the plant from which they were isolated or chromosomal marker analysis of the plant from which they were isolated.

The methods can be used to produce nucleic acids from the plant, plant part, seed or cell, which nucleic acids can be, for example, analyzed to produce data. The data can be recorded and used in a plant breeding program or can be used to develop markers or used in marker assisted selection to identify plants for use in subsequent crosses to generate progeny plants containing the trait of interest. The nucleic acids from the disrupted cell, the disrupted plant, plant part, plant cell or seed or the nucleic acids following isolation or separation can be contacted with primers and nucleotide bases, and/or a polymerase to facilitate PCR sequencing or marker analysis of the nucleic acids. In some examples, the nucleic acids produced can be sequenced or contacted with markers to produce a genetic profile, a molecular profile, a marker profile, a haplotype, or any combination thereof. In some embodiments, the genetic profile or nucleotide sequence is recorded on a computer readable medium. In other embodiments, the methods may further comprise using the nucleic acids produced from plants, plant parts, plant cells or seeds in a plant breeding program, for example in making crosses, selection and/or advancement decisions in a breeding program. Crossing includes any type of plant breeding crossing method, including but not limited to crosses to produce hybrids, outcrossing, selfing, backcrossing, locus conversion, introgression and the like.

Favorable genotypes and or marker profiles, optionally associated with a trait of interest, may be identified by one or more methodologies. In some embodiments one, two, three, or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes. In some embodiments, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some embodiments, no target-specific probe is needed, for example by using sequencing technologies, including but not limited to next-generation sequencing methods such as sequencing by synthesis (e.g., Roche 454 pyrosequencing, Illumina Genome Analyzer, and Ion Torrent PGM or Proton systems), sequencing by ligation (e.g., SOLID from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme, and expression methods. In some embodiments, no reference genome sequence is needed in order to complete the analysis.

### Molecular Markers

“Marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one, two, three, or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus.

“Marker” also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD). Markers within and between locations spanning or contained within or between any of the start and end positions of the sequences shown in Table 1 can be used that are on the same chromosome. For example, the start and end position can be of the same sequence or, for example, the start position of can be for one sequence and the end position be for another sequence on the same chromosome.

A favorable allele of a marker is the allele of the marker that co-segregates with a desired phenotype (e.g., kernel color). As used herein, a QTL marker has a minimum of one favorable allele, although it is possible that the marker might have two, three, or more favorable alleles found in the population. Any favorable allele of that marker can be used advantageously for the identification and generation of white or yellow corn plant lines. Optionally, at least 1, at least 2, at least 3, at least 4, at least 5 or at least 6 or more favorable allele(s) and less than 25, less than 20, less than 15, less than 10, less than 9, less than 8, less than 7 or less than 6 favorable alleles of different markers are identified in, or introgressed into a plant, and can be selected for or against during marker assisted selection in methods encompassing one or more than identification, selection and crossing of plants and plant breeding. Desirably, plants, seeds, lines or germplasm are identified that have at least one, at least two, at least three or at least four such favorable alleles that positively correlate with kernel color. Marker alleles that co-segregate with kernel color can also be used with the methods described herein. Marker alleles that are associated with traits common in non-white kernels, such as yellow or brown traits in the kernel or a red cob color can be used to identify and select for white or yellow kernelled corn plants having a desirable phenotype. Such alleles can be used for exclusionary purposes during breeding to identify alleles that negatively correlate with white or yellow kernel color, to eliminate yellow or white kernelled plants, seeds, lines or germplasm, or their common traits from subsequent rounds of breeding.

More tightly linked markers with a DNA locus influencing a phenotype, such as kernel color, can be used in the methods described herein, as the likelihood of a recombination event unlinking the marker and the locus decreases. Markers containing the causal mutation for a trait, or that are within the coding sequence of a causative gene, can be used as no recombination is expected between them and the sequence of DNA responsible for the phenotype.

Genetic markers are distinguishable from each other (as well as from the plurality of alleles of any one particular marker) on the basis of polynucleotide length and/or sequence. A large number of corn molecular markers are known in the art, and are published or available from various sources, such as the MaizeGDB internet resource. In general, any differentially inherited polymorphic trait (including a nucleic acid polymorphism) that segregates among progeny is a potential genetic marker.

In some embodiments, one or more marker alleles are selected for in a single plant or a population of plants. In these methods, plants are selected that contain favorable alleles from one or more than one kernel color markers, or alternatively, favorable alleles from one or more than one kernel color markers are introgressed into a desired germplasm. One of skill recognizes that the identification of favorable markers correlating with white or yellow kernel color can be determined for the particular germplasm under study following the teachings described herein. One of skill recognizes that the identification and use of such favorable alleles is well within the scope of the methods and compositions described herein. Furthermore still, identification of favorable marker alleles in plant populations other than the populations used or described herein is well within the scope of the methods and compositions described herein.

### Marker Detection

In some aspects, methods disclosed herein utilize an amplification step to detect/genotype a marker locus, but amplification is not always a requirement for marker detection (e.g., Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

“Amplifying,” in the context of nucleic acid amplification, is any process whereby additional copies of a selected nucleic acid (or a transcribed form thereof) are produced. In some embodiments, an amplification-based marker technology is used wherein a primer or amplification primer pair is admixed with genomic nucleic acid isolated from the first plant or germplasm, and wherein the primer or primer pair is complementary or partially complementary to at least a portion of the marker locus and is capable of initiating DNA polymerization by a DNA polymerase using the plant genomic nucleic acid as a template. The primer or primer pair is extended in a DNA polymerization reaction having a DNA polymerase and a template genomic nucleic acid to generate at least one amplicon. In other embodiments, plant RNA is the template for the amplification reaction. In some embodiments, the QTL marker is a SNP type marker, and the detected allele is a SNP allele, and the method of detection is allele specific hybridization (ASH).

In general, the majority of genetic markers rely on one or more properties of nucleic acids for their detection. Typical amplification methods include various polymerase-based replication methods, including the polymerase chain reaction (PCR), ligase mediated methods such as the ligase chain reaction (LCR) and RNA polymerase-based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like). A “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A “template nucleic acid” is a nucleic acid that serves as a template in an amplification reaction (e.g., a polymerase-based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as, for example, TAQMAN probes, can also be performed as described herein. These probes are composed of short (e.g., 18-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan probes are oligonucleotides that have a label and a quencher, where the label is released during amplification by the exonuclease action of the polymerase used in amplification, providing a real time measure of amplification during synthesis.

In some embodiments, the presence or absence of a molecular marker is determined simply through nucleotide sequencing of the polymorphic marker region. This method is readily adapted to high throughput analysis as are the other methods noted above, e.g., using available high throughput sequencing methods such as sequencing by hybridization.

In some embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors.

While exemplary markers are provided herein, any of the aforementioned marker types can be employed in the context of the methods, compositions and plants disclosed herein to identify chromosome intervals encompassing genetic elements that contribute to superior agronomic traits (such as white or yellow corn kernels).

### Probes and Primers

In general, synthetic methods for making oligonucleotides, including probes, primers, molecular beacons, PNAs, LNAs (locked nucleic acids), etc., are well known. For example, oligonucleotides can be synthesized chemically according to a solid phase phosphoramidite triester method. Oligonucleotides, including modified oligonucleotides, can also be ordered from a variety of commercial sources.

Nucleic acid probes to the marker loci can be cloned and/or synthesized. Any suitable label can be used with a probe such as described herein. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes as described herein be limited to any particular size.

In some embodiments, the molecular markers useful in the methods disclosed herein are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. As used in the art, PCR primers used to amplify a molecular marker are sometimes termed “PCR markers” or simply “markers.” It will be appreciated that, although many specific examples of primers are provided herein, suitable primers to be used can be designed using any suitable method. It is not intended that the methods and compositions described herein be limited to any particular primer or primer pair. In some embodiments, the primers which can be used are radiolabeled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons. It is not intended that the primers described herein be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, at least 50 nucleotides in length, at least 100 nucleotides in length, at least 200 nucleotides in length and less than 5000 nucleotides in length, less than 4000 nucleotides in length, less than 3000 nucleotides in length, less than 2000 nucleotides in length, less than 1000 nucleotides in length, less than 500 nucleotides in length, less than 400 nucleotides in length, less than 300 nucleotides in length, less than 200 nucleotides in length, or less than 100 nucleotides in length. Marker alleles in addition to those recited herein may also be used.

### Linkage Analysis

“Linkage”, or “genetic linkage,” is used to describe the degree with which one marker locus is associated with another marker locus or some other locus (for example, a kernel color locus). A marker locus may be located within a locus to which it is genetically linked. For example, if locus A has genes “A” or “a” and locus B has genes “B” or “b” and a cross between parent 1 with AABB and parent 2 with aabb will produce four possible gametes where the genes are segregated into AB, Ab, aB and ab. The null expectation is that there will be independent equal segregation into each of the four possible genotypes, i.e., with no linkage ¼ of the gametes will of each genotype. Segregation of gametes into genotypes differing from ¼ is attributed to linkage. As used herein, linkage can be between two markers, or alternatively between a marker and a phenotype. A marker locus may be genetically linked to a trait, and in some cases a marker locus genetically linked to a trait is located within the allele conferring the trait. A marker may also be causative for a trait or phenotype, for example a causative polymorphism. The degree of linkage of a molecular marker to a phenotypic trait (e.g., a QTL) is measured, e.g., as a statistical probability of co-segregation of that molecular marker with the phenotype.

As used herein, “closely linked” means that the marker or locus is within 20 cM or less than about 20 cM, for instance within 10 cM or less than about 10 cM, within 5 cM or less than about 5 CM, within 1 cM or less than about 1 cM, within 0.5 CM or less than about 0.5 CM, or within 0.25 CM or less than about 0.25 CM of the identified locus associated with kernel color.

As used herein, the linkage relationship between a molecular marker and a phenotype is the statistical likelihood that the particular combination of a phenotype and the presence or absence of a particular marker allele is random. Thus, the lower the probability score, the greater the likelihood that a phenotype and a particular marker will cosegregate. In some embodiments, a probability score of 0.05 (p=0.05, or a 5% probability) of random assortment is considered a significant indication of co-segregation. However, the methods and compositions described herein are not limited to this particular standard, and an acceptable probability can be any probability of less than 50% (p<0.5). For example, a significant probability can be less than 0.25, less than 0.20, less than 0.15, or less than 0.1. The phrase “closely linked,” means that recombination between two linked loci occurs with a frequency of equal to or less than about 10% (i.e., are separated on a genetic map by not more than 10 cM). In one aspect, any marker described herein is linked (genetically and physically) to any other marker that is at or less than 50 cM distant. In another aspect, any marker described herein is closely linked (genetically and physically) to any other marker that is in close proximity, e.g., at or less than 10 cM distant. Two closely linked markers on the same chromosome can be positioned less than 9, less than 8, less than 7, less than 6, less than 5, less than 4, less than 3, less than 2, less than 1, less than 0.75, less than 0.5 or less than 0.25 cM or less from each other.

Classical linkage analysis provides a statistical description of the relative frequencies of cosegregation of different traits. Linkage analysis is the well characterized descriptive framework of how traits are grouped together based upon the frequency with which they segregate together. That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. The further apart on a chromosome the genes reside, the less likely they are to segregate together, because homologous chromosomes recombine during meiosis. Thus, the further apart on a chromosome the genes reside, the more likely it is that there will be a crossing over event during meiosis that will result in the marker and the DNA sequence responsible for the trait the marker is designed to track segregating separately into progeny. A common measure of linkage is the frequency with which traits co-segregate.

Linkage analysis can be used to determine which polymorphic marker allele demonstrates a statistical likelihood of co-segregation with the kernel color phenotype (thus, a “kernel color marker allele”). Following identification of a marker allele for co-segregation with the kernel color phenotype, it is possible to use this marker for rapid, accurate screening of plant lines for the kernel color allele without the need to grow the plants through their life cycle and await phenotypic evaluations, and furthermore, permits genetic selection for the particular kernel color allele even when the molecular identity of the actual QTL is unknown. Tissue samples can be taken, for example, from the endosperm, embryo, or mature/developing plant and screened with the appropriate molecular marker to rapidly determine determined which progeny contain the desired genetics. Linked markers also remove the impact of environmental factors that can influence phenotypic expression.

Because chromosomal distance is approximately proportional to the frequency of crossing over events between traits, there is an approximate physical distance that correlates with recombination frequency. Marker loci are themselves traits and can be assessed according to standard linkage analysis by tracking the marker loci during segregation. Thus, in the context described herein, one cM is equal to a 1% chance that a marker locus will be separated from another locus (which can be any other trait, e.g., another marker locus, or another trait locus that encodes a QTL), due to crossing over in a single generation.

When referring to the relationship between two genetic elements, such as a genetic element contributing to kernel color and a proximal marker, “coupling” phase linkage indicates the state where the “favorable” allele at the kernel color locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for kernel color) is physically linked with an “unfavorable” allele at the proximal marker locus, and the two “favorable” alleles are not inherited together (i.e., the two loci are “out of phase” with each other).

### Genetic Mapping

A “genetic map” is the relationship of genetic linkage among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map. A “genetic recombination frequency” is the frequency of a crossing over event (recombination) between two genetic loci. Recombination frequency can be observed by following the segregation of markers and/or traits following meiosis. In some cases, two different markers can have the same genetic map coordinates. In that case, the two markers are in such close proximity to each other that recombination occurs between them with such low frequency that it is undetected.

Genetic maps are graphical representations of genomes (or a portion of a genome such as a single chromosome) where the distances between markers are measured by the recombination frequencies between them. Plant breeders use genetic maps of molecular markers to increase breeding efficiency through marker-assisted selection, a process where selection for a trait of interest is not based on the trait itself but rather on the genotype of a marker linked to the trait. A molecular marker that demonstrates reliable linkage with a phenotypic trait provides a useful tool for indirectly selecting the trait in a plant population, especially when accurate phenotyping is difficult, slow, or expensive.

In general, the closer two markers or genomic loci are on the genetic map, the closer they lie to one another on the physical map. A lack of precise proportionality between cM distances and physical distances can exist due to the fact that the likelihood of genetic recombination is not uniform throughout the genome; some chromosome regions are cross-over “hot spots,” while other regions demonstrate rarer recombination events, if any.

Genetic mapping variability can also be observed between different populations of the same crop species. In spite of this variability in the genetic map that may occur between populations, genetic map and marker information derived from one population generally remains useful across multiple populations in identification of plants with desired traits, counter-selection of plants with undesirable traits and in guiding marker assisted selection.

As one of skill in the art will recognize, recombination frequencies (and as a result, genetic map positions) in any particular population are not static. The genetic distances separating two markers (or a marker and a QTL) can vary depending on how the map positions are determined. For example, variables such as the parental mapping populations used, the software used in the marker mapping or QTL mapping, and the parameters input by the user of the mapping software can contribute to the QTL marker genetic map relationships. However, it is not intended that the embodiments described herein be limited to any particular mapping populations, use of any particular software, or any particular set of software parameters to determine linkage of a particular marker or chromosome interval with the white or yellow kernel color phenotype. It is well within the ability of one of ordinary skill in the art to extrapolate the novel features described herein to any gene pool or population of interest and using any particular software and software parameters. Indeed, observations regarding genetic markers and chromosome intervals in populations in addition to those described herein are readily made using the teachings described herein.

### Association Mapping

Association or LD mapping techniques aim to identify genotype-phenotype associations that are significant. It is effective for fine mapping in outcrossing species where frequent recombination among heterozygotes can result in rapid LD decay. LD is non-random association of alleles in a collection of individuals, reflecting the recombinational history of that region. Thus, LD decay averages can help determine the number of markers necessary for a genome-wide association study to generate a genetic map with a desired level of resolution.

Large populations are better for detecting recombination, while older populations are generally associated with higher levels of polymorphism, both of which contribute to accelerated LD decay. However, smaller effective population sizes tend to show slower LD decay, which can result in more extensive haplotype conservation. Understanding of the relationships between polymorphism and recombination is useful in developing strategies for efficiently extracting information from these resources. Association analyses compare the plants' phenotypic score with the genotypes at the various loci. Subsequently, any suitable maize genetic map (for example, a composite map) can be used to help observe distribution of the identified QTL markers and/or QTL marker clustering using previously determined map locations of the markers.

### Marker Assisted Selection

“Introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A primary motivation for development of molecular markers in crop species is the potential for increased efficiency in plant breeding through marker-assisted selection. Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. Disclosed herein are methods and compositions to identify plants that produce a white or yellow kernel phenotype by identifying plants having a specified allele that is linked to Bn7, Y1, Bn1, Wc1, and/or P1-ww.

In general, marker-assisted selection uses polymorphic markers that have been identified as co-segregating with a kernel color trait. Such markers are presumed to map near a gene or genes that give the plant its kernel color, and are considered indicators for the desired trait, and are termed QTL markers. Plants are tested for the presence or absence of a desired allele in the QTL marker.

Identification of plants or germplasm that include a marker locus or marker loci linked to a kernel color trait or traits provides a basis for performing marker-assisted selection, such as described herein. Plants that comprise favorable markers or favorable alleles are selected for, while plants that comprise markers or alleles that are negatively correlated with white or yellow kernel color can be selected against. Desired markers and/or alleles can be introgressed into plants having a desired (e.g., elite or exotic) genetic background to produce an introgressed white or yellow kernelled plant or germplasm. In some aspects, it is contemplated that a plurality of kernel color markers are sequentially or simultaneous selected and/or introgressed. The combinations of kernel color markers that are selected for in a single plant is not limited and can include any combination of markers disclosed herein, or any marker linked to the markers disclosed herein, or any markers located within the QTL intervals defined herein.

In some embodiments, a white or yellow kernelled first corn plant or germplasm (the donor) is crossed with a second corn plant or germplasm (the recipient, e.g., an elite or exotic corn, depending on characteristics that are desired in the progeny) to create an introgressed corn plant or germplasm as part of a breeding program designed to improve white kernel color of the recipient corn plant or germplasm. In some aspects, the recipient plant contains one or more kernel color loci, which can be the same or different qualitative or quantitative trait loci from the donor plant. In another aspect, the recipient plant and/or the donor plant contains a transgene.

Marker-assisted selection permits selecting for desired phenotypes and for introgressing desired traits into cultivars (e.g., introgressing desired traits into elite lines) following an initial cross or from a provided population. Marker-assisted selection is easily adapted to high throughput molecular analysis methods that can quickly screen large numbers of plant or germplasm genetic material for the markers of interest and is much more cost effective than raising and observing plants for visible traits.

When a population is segregating for multiple loci affecting one or multiple traits, e.g., multiple loci involved in white or yellow kernel color, or multiple loci each involved in kernel color, the efficiency of marker-assisted selection compared to phenotypic screening becomes even greater, because all of the loci can be evaluated together from a single sample of DNA.

Introgression of Bn7, Y1, and/or Wc1 Loci Using Marker-Assisted Selection

The introgression of one or more desired loci from a donor line into another can be achieved via repeated backcrossing to a recurrent parent accompanied by selection to retain one or more Bn7, Bn1, Y1, and/or Wc1 loci and optionally loci for white cob color such as P1 from the donor parent. Markers associated with Bn7, Y1, and/or Wc1 and optionally Bn1 and cob color are assayed in progeny and those progeny with one or more Bn7, Y1, and/or Wc1 markers and optionally markers for white cob color are selected for advancement. In another aspect, one or more markers can be assayed in the progeny to select for plants with the genotype of the agronomically elite parent. The methods and compositions described herein anticipate that trait introgression activities may be achieved through the use of double haploids or through classic introgression, wherein progeny are crossed to the recurrent (agronomically elite) parent or selfed through multiple generations. Selections are made based on the presence of one or more Bn7, Y1, and/or Wc1 markers and optionally cob color markers and can also be made based on the recurrent parent genotype, wherein screening is performed on a genetic marker and/or phenotype basis. In some embodiments, markers disclosed herein can be used in conjunction with other markers, such as at least one on each chromosome of the corn genome, to track the introgression of Bn7, Y1, and/or Wc1 loci in the desired allele state such as white or whiter pericarp, endosperm, aleurone kernel, and/or cob into elite germplasm. In another embodiment, QTLs associated with Bn7, Y1, and/or Wc1 will be useful in conjunction with SNP or other molecular markers such as indel or copy number variation markers including those envisaged or disclosed herein to combine quantitative and qualitative traits in the same plant. In some embodiments, markers associated with P1-ww are optionally assayed and used in selection in conjunction with other markers disclosed herein. It is within the scope of this invention to utilize the methods and compositions for trait integration of Bn7, Y1, and/or Wc1 and/or P1-ww. It is contemplated by the inventors that the methods and compositions described herein will be useful for developing commercial varieties with white kernel color and an agronomically elite phenotype. In performing screening for double haploids or for introgression a marker that correctly identifies the desired genotype in all or almost all cases is desirable as efficiency in advancing only desired segregants from a segregating population. The methods disclosed herein correctly identify the desired genotype (including zygosity states of homozygous or heterozygous) at least 90%, at least 95%, at least 96%, at least 97%, at least 98%, at least 99% or at least 99.5% of the time facilitating efficient plant breeding and production of desirable plants through the doubled haploid process.

Provided are methods which include a step of separating seeds identified as non-yellow, yellow or white, or a combination thereof from a population of seeds. Such a step may include the use of a color sorter device, such as an optical color sorter. Provided are methods in which the seeds are separated by passing the seeds through a color sorter to separate the seeds based on color. Any color may be used to sort seeds depending on the desired genotype or phenotype, such as white, lemon, brown or yellow (light yellow or dark yellow). In some embodiments, yellow seeds containing at least one dominant Y1 allele are separated from the population.

In some embodiments, the marker assays disclosed herein can be used to discriminate at least 50%, at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% samples having of the desired allele state, such as white or whiter pericarp, endosperm, aleurone, kernel and/or cob from plants, seeds or plant parts assayed for any of the markers disclosed herein, including one or more of those for Bn7, Y1, Wc1 and/or P1-ww. The plants, seeds or plant parts can be separated after being identified or discriminated, with the plants, seeds and plant parts identified and separated used in crossing and breeding such as in methods described herein.

In some embodiments, the markers disclosed herein and markers useful in methods disclosed herein are within 0.1 cM, 0.2 CM, 0.3 CM, 0.4 CM, 0.5 CM, 1 CM, 2 CM, 3 CM, 4 CM, 5 CM, 10 cM or 20 cM of a sequence causal or associative with a phenotype, such as white or whiter pericarp, endosperm, aleurone, kernel and/or cob, including one or more of those for Bn7/bn7, Y1/y1, Wc1 and/or P1-ww, such as any of the sequences disclosed herein or provided in the sequence listing.

In an aspect, the methods and compositions described herein can be used on any plant. In another aspect, the plant is selected from the genus Zea. In another aspect, the plant is selected from the species Zea mays. In a further aspect, the plant is selected from the subspecies Zea mays L. ssp. mays. In an additional aspect, the plant is selected from the group Zea mays L. subsp. mays Indentata, otherwise known as dent corn. In another aspect, the plant is selected from the group Zea mays L. subsp. mays Indurata, otherwise known as flint corn. In an aspect, the plant is selected from the group Zea mays L. subsp. mays Saccharata, otherwise known as sweet corn. In another aspect, the plant is selected from the group Zea mays L. subsp. mays Amylacea, otherwise known as flour corn. In a further aspect, the plant is selected from the group Zea mays L. subsp. mays Everta, otherwise known as popcorn. Zea plants include hybrids, inbreds, partial inbreds, or members of defined or undefined populations.

Plants useful in the methods and compositions disclosed herein may include transgenes or locus conversions. Any sequences, such as DNA, whether from a different species or from the same species, which have been stably inserted into a genome using transformation are referred to herein collectively as “transgenes” and/or “transgenic events”. Transgenes or locus conversions can be moved from one genome to another using breeding techniques which may include crossing, backcrossing or double haploid production.

Transgenes and transformation methods facilitate engineering of the genome of plants to contain and express heterologous genetic elements, such as foreign genetic elements, or additional copies of endogenous elements, or modified versions of native or endogenous genetic elements in order to alter at least one trait of a plant in a specific manner. Any sequences, such as DNA, whether from a different species or from the same species, which have been stably inserted into a genome using transformation are referred to herein collectively as “transgenes” and/or “transgenic events”. Transgenes can be moved from one genome to another using breeding techniques which may include crossing, backcrossing or double haploid production. In some embodiments, a transformed variant of «Product» may comprise at least one transgene but could contain at least 1, 2, 3, 4, 5, 6, 7, 8, 9, 10 and/or no more than 15, 14, 13, 12, 11, 10, 9, 8, 7, 6, 5, 4, 3, or 2. Transformed versions of the claimed maize variety «Product»> as well as hybrid combinations containing and inheriting the transgene thereof are provided. F1 hybrid seed are provided which are produced by crossing a different maize plant with maize variety «Product» comprising a transgene introduced into maize variety «Product» by backcrossing or genetic transformation and which transgene is inherited by the F1 hybrid seed.

Numerous methods for plant transformation have been developed, including biological and physical plant transformation protocols. In addition, expression vectors and in vitro culture methods for plant cell or tissue transformation and regeneration of plants are available.

In general, methods to transform, modify, edit or alter plant endogenous genomic DNA include altering the plant native DNA sequence or a pre-existing transgenic or other heterologous sequence including regulatory elements, transgene-genomic junction sequences, coding and non-coding sequences. These methods can be used, for example, to target nucleic acids to pre-engineered target recognition sequences in the genome. Such pre-engineered target sequences may be introduced by genome editing or modification. As an example, a genetically modified or genome edited plant variety can be generated using “custom” or engineered endonucleases such as meganucleases produced to modify plant genomes (see e.g., WO/2009/114321; Gao et al. (2010) Plant Journal 1:176-187); zinc finger nucleases; a transcription activator-like (TAL) effector-DNA modifying enzyme (TALE or TALEN) (see e.g., US20110145940). Site-specific modification of plant genomes can also be performed using the CRISPR (clustered regularly interspaced short palindromic repeats)/Cas (CRISPR-associated) system. See e.g., Cas9/guide RNA-based system that allows targeted cleavage of genomic DNA guided by a customizable small noncoding RNA in plants (see e.g., PCT Publication Number WO/2015/026883A1) and Cas12f1 miniature CRISPR system that is used to introduce site-specific changes in the plant genome (see e.g., U.S. Pat. No. 10,934,536B2).

Plant transformation methods may involve the construction of an expression vector. Such a vector comprises a DNA sequence that contains a gene under the control of or operatively linked to a regulatory element, for example a promoter. The vector may contain one or more genes and one or more regulatory elements.

A transgenic event which has been stably engineered into the germ cell line of a particular maize plant using transformation techniques, could be moved into the germ cell line of another variety using traditional breeding techniques that are well known in the plant breeding arts or through targeted/directed cleavage of the transgenic loci using molecular trait introgression methods, such as targeted recombination including directed homology dependent recombination (HDR). For example, a backcrossing approach is commonly used to move a transgenic event from a transformed maize plant to another variety, and the resulting progeny would then comprise the transgenic event(s). Also, if an inbred variety was used for the transformation then the transgenic plants could be crossed to a different inbred in order to produce a transgenic hybrid maize plant.

Various genetic elements can be introduced into the plant genome using transformation. These elements include, but are not limited to genes, coding sequences; inducible, constitutive, and tissue specific promoters; enhancing sequences; and signal and targeting sequences. For example, see the traits, genes and transformation methods listed in U.S. Pat. Nos. 6,118,055 and 6,284,953. In addition, transformability of a variety can be increased by introgressing the trait of high transformability from another variety known to have high transformability, such as Hi-II. See US Patent Publication US2004/0016030.

Plant breeding techniques which can be used in a maize plant breeding program and with the methods and compositions disclosed herein include, but are not limited to, recurrent selection, mass selection, bulk selection, mass selection, backcrossing, pedigree breeding, open pollination breeding, restriction fragment length polymorphism enhanced selection, genetic marker enhanced selection, making double haploids, and transformation. Often combinations of these techniques are used. There are many analytical methods available to evaluate the result of a cross. The oldest and most traditional method of analysis is the observation of phenotypic traits, but genotypic analysis may also be used.

Methods for producing a maize plant by crossing a first parent maize plant with a second parent maize plant are provided. The maize plant may be for example an inbred or a hybrid. The other parent may be any other maize plant, such as another inbred or hybrid variety or a plant that is part of a synthetic or natural population. Any such methods may be used such as selfing, sibbing, backcrosses, mass selection, pedigree breeding, bulk selection, hybrid production, crosses to populations, and the like. These methods are well known in the art and some of the more commonly used breeding methods are described below.

Pedigree breeding starts with the crossing of two genotypes. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically, in the pedigree method of breeding, five or more successive filial generations of selfing and selection is practiced: F1→F2; F2→F3; F3→F4; F4→F5, etc. To obtain an inbred line following a cross, a number of backcrosses can be performed. For example, the elite genetics of a yellow line carrying at least one Y1 allele from a breeding program producing yellow inbreds, such as yellow dent inbreds for use as a crop, can be converted to a white inbred (such as having white kernels and white cob) by crossing with one or more lines carrying the alleles contributing to whiteness and selecting for non-yellow lines following a number of backcrosses, such as BC1, BC2, BC3, BC4, BC5 etc. The methods disclosed herein can be used to hold alleles contributing to white corn in a heterozygous state, thereby facilitating obtaining genetics from the elite yellow parent. After a sufficient amount of inbreeding, successive filial generations will serve to increase seed of the developed inbred. Provided, for example, are inbred varieties comprising homozygous alleles at about 95% or more of its loci. The inbred line produced in the methods disclosed herein can comprise at least 50%, at least 60%, at least 70%, at least 80%, at least 85%, at least 90%, at least 95, at least 96%, at least 97%, at least 98% or at least 99% of the alleles found in the yellow elite inbred line but contain the alleles contributing to whiteness disclosed herein.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross pollinated with each other to form progeny for another population. This population is planted, and again superior plants are selected to cross pollinate with each other. Recurrent selection is a cyclical process and can be repeated as many times as desired. The objective of recurrent selection is to improve the traits of a population. The improved population can then be used as a source of breeding material to obtain inbred varieties to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program. The single seed descent (SSD) method is a modified form of bulk breeding in which only one seed is selected randomly from each plant in F1 and subsequent generations. The selected seed is bulked and used to grow the next generation.

The production of double haploids (DH) can also be used for the development of new lines such as inbreds. Double haploids are produced by the doubling of a set of chromosomes (1N) in a haploid plant derived from a heterozygous plant to produce a completely homozygous individual. This can be advantageous because the process omits the generations of selfing needed to obtain a homozygous plant from a heterozygous source.

Haploid induction systems have been developed for various plants to produce haploid tissues, plants and seeds. The haploid induction system can produce haploid plants from any genotype by crossing a selected variety (as female) with an inducer variety. Such inducer varieties for maize include Stock 6, RWS, KEMS, KMS and ZMS, and indeterminate gametophyte (ig) mutation.

The ability to efficiently produce doubled haploids (DHs) from crosses between white and yellow inbred lines for use in white corn breeding is often inefficient due to the need to select for several recessive traits at one time and the interaction of Bn7, Wc1, and Y1 on the kernel phenotype and resulting lines. Populations produced or derived from a cross between an elite yellow kernelled corn variety and a white corn variety or a variety carrying alleles contributing to whiter corn, or populations containing a mixture of alleles contributing to white corn and yellow corn can be used as a starting point for breeding methods disclosed herein, including double haploid production. The addition of markers for key white corn traits such as P1-ww would enable efficient recovery of acceptable white lines through marker-assisted preselection of F2 kernels to enter into the doubled haploid process by reducing error associated with visual phenotyping. Using a visual selection process either requires selfing to F3 to observe line cob color before submitting, which is inefficient, or producing a proportion of red cobbed lines which do not have the desired white cob phenotype and are unacceptable to the market. In addition to a need to select for cob color, error associated with phenotyping kernel color results in a large number of Bn7/Bn7 DHs, reducing the efficiency of DH production. In a white x yellow kernelled population, marker preselection of F2 kernels for bn7 and y1 homozygotes, coupled with visual or mechanical selection for y1 homozygotes (white kernels) is expected to result in production of doubled haploids with uniformly acceptable white kernel and white cob color, thus enabling increased genetic gain for non-color traits.

The following examples illustrate particular aspects of the disclosure and are not intended in any way to limit the disclosure.

### EXAMPLES

### Example 1. Marker Development for y1 Gene

A panel of 1600 proprietary corn inbred lines representing both stiff stalk and non-stiff stalk pools were sequenced and single nucleotide polymorphism's (SNP's) and small insertion and deletion (indel) polymorphisms were determined by comparing against B73 public reference genome v2. This panel included 1528 yellow lines and 141 white lines from Africa, Latin America, and North America. The phytoene synthase gene (y1; GRMZM2G300348) on chromosome 6 (SEQ ID NO: 109 encoding SEQ ID NO: 110) was investigated for associated polymorphisms for marker development.

The TaqMan assays were developed as follows: Primers were designed using the primer 3 software (found online at bioprod.phibred.com/primer3/cgi-bin/primer3_www.cgi). Probes were designed using Primer Express Software. 1.5 μl of the 1:100 DNA dilution was used in the assay mix. 18 μM of each probe, and 4 M of each primer was combined to make each assay. 13.6 μl of the assay mix was combined with 1000 μl of KASP Master Mix. A Meridian (Kbio) liquid handler dispensed 1.3 μl of the mix onto a 1536 plate containing ~6 ng of dried DNA. The plate was sealed with a Phusion laser sealer and thermocycled using a Kbio Hydrocycler with the following conditions: 94 °C for 15 min, 40 cycles of 94 °C for 30 sec, 60C for 1 min. The excitation at wavelengths 485 (FAM) and 520 (VIC) was measured with a Pherastar plate reader. The values were normalized against ROX and plotted and scored on scatterplots utilizing the KRAKEN software.

Three SNPs, two in exon 1 C104CVA-001-Q001 (SEQ ID NO: 9) and C104CVB-001-Q001 (SEQ ID NO: 14) and one upstream in the promoter region C104CWM-001-Q034 (SEQ ID 19; 2259 bp upstream of the gene start) were identified and were found to discriminate between white and yellow kernels in global germplasm panels.

C104CVN-001-Q001 (SEQ ID NO: 5), which contains a 378 bp indel, was converted into a TaqMan assay, that combines two separate assay amplifications. The first amplification assay M (mutant) comprises SEQ ID NOs. 6, 7, 8 and detects the presence of the insertion, while the W (wildtype) assay comprises SEQ ID NOs. 2, 3, 4 and detects the wild type or lack of insertion. Together these two assays in one well of a TaqMan PCR reaction, were found to act as co-dominant markers to discriminate the white and yellow kernels in all zygosity states.

TaqMan assays were developed for the SNPs and indels contained in C104CVA-001, C104CVB-001, C104CVN-001-Q001 and C104CWM-001 and were validated for technical performance on a panel of inbreds composed of white and yellow lines, and on F2 populations developed from a cross of white x yellow kernel parents.

When compared, to the SNP in C104CVP-001-Q001 (SEQ ID NO: 24), which was found to have only an 86% association for discriminating white and yellow kernels in our tested panel, the SNPs assays in C104CVA-001-Q001, C104CVB-001-Q001 and C104CWM-001-Q034 and the indel assay comprising SEQ ID NOs 2-4 and 6-8 were found to have a 100% discrimination association.

TABLE 2

| B73 V2.1 | | | |
|---|---|---|---|
| Physical | | | |
| co-ordinate | Marker Name | Location | SEQ ID NO: |
| 85204671 | C104CVA-001-Q001 | 1st Exon | SEQ ID NO: 9 |
| 85204725 | C104CVB-001-Q001 | 1st Exon | SEQ ID NO: 14 |
| 85206146 | C104CWM-001-Q034 | Promoter | SEQ ID NO: 19 |
| 85205361 | C104CVN-001-Q001 and | Promoter | SEQ ID NOS: 1 |
| | C104CVM-001-Q001 | | and 5 |

### Example 2. Marker Development for White Cap (Wc1) Gene

Ccd1 (Carotenoid cleavage dioxygenase 1) reference locus is present in corn and teosinte. In Wc lines a macro-transposon with 1 to 23 copies of Ccd1 is found on chromosome 9 at about position 157,485,000. Each repeat of the transposon contains three genes including Ccd1 (coding sequence SEQ ID NO: 111). At least 3 copies provide a white kernel color, with more copies providing a brighter whiter kernel color. An insertion site sequence at the macro-transposon was used to develop a presence/absence high-throughput real-time PCR copy number variation (CNV) assay to estimate the copy number of Ccd1 and to detect the presence or absence of the Wc1 locus. The Ccd1 marker sequence portion for the CNV assay was run using the probe SEQ ID NO: 37 and the primers SEQ ID NOS: 38 and 39. Alcohol dehydrogenase (Adh1) was used as an internal control gene and B73, which contains a single copy of Ccd1, was used as another control to optimize the copy number estimates, using probe SEQ ID NO: 40 and the primers SEQ ID NOs: 41 and 42. Primers for Adh-1 endogenous control were designed using the primer 3 software (found online at bioprod.phibred.com/primer3/cgi-bin/primer3_www.cgi). The probe was designed using Primer Express Software. Ccd1 probe was labelled with 6-FAM Tamra dye, while Adh1 was labelled with VIC dye. 18 μM of each probe, and 4 μM of each primer was combined to make each assay. 13.6 ul of the assay mix was combined with 1000 ul of Applied Biosystems TaqMan Master Mix. 10 μL was pipetted into a 96 well plate containing 10 ng of dried DNA. The plate was sealed and thermocycled and read in an Applied Biosystems Viia7 with the following conditions: 94C for 5 min, 40 cycles of 94C for 1 min, 58C for 1 min, 72 for 3 min. The excitation at wavelengths 485 (FAM) and 520 (VIC) was measured at each cycle within the Viia7. The values were normalized against ROX, and copy number was calculated utilizing the CopyCaller software. We characterized a panel of 252 inbred white lines and 10 yellow lines as single copy controls with this genotyping assay. The copy numbers ranged between 1-22. The yellow lines predominantly lack the macro-transposon repeat, whereas North American white lines contains multiple copies. The Copy Number Variety assay was applied to select high copy number white lines as parents to initiate breeding crosses with yellow lines and to maintain the high copy number in marker assisted selection in white corn development and to facilitate selected introgression of Wc1 into those germplasm segments to further improve the white color.

### Example 3. Fine Mapping of a Negative Modifier of White Color & Marker Development

A negative modifier of the kernel color named Bn7 was discovered through a genome wide association study and fine mapping following biparental crosses. Presence of wild type Bn7 over bn7 results in lemon-colored kernels (pale yellow), that are not commercially acceptable. A genome-wide association (GWAS) study was conducted using a panel of 1600 lines used in Example 1 to locate the Bn7 location in the genome. A major peak on the chromosome 7 was discovered at ~120.6 cM, that discriminated yellow lines from the white lines. We did two genetic experiments to investigate this finding. First, the dominant mode of inheritance of Bn7 was tested. Second, recombination around this region was exploited to fine map Bn7 and develop marker assisted selection against it.

The maize brown aleurone layer is visually observable in the kernel in the fixed presence of the white allele of Y1, i.e., y1/y1, based on differences between pale yellow-comprising at least one Bn7 allele (Bn7/_) and true white (bn7/bn7) kernels, but is not visually observable in the present of lines comprising at least one Y1 allele. To determine Bn7 allele status of yellow lines fixed for Y1, i.e., Y1/Y1, the Y1/Y1 lines were crossed to a genetic stock 602C (available from the Maize Genetic Cooperation Stock Center) with a known genotype y1/y1 bn7/bn7 and then self-pollinated for one generation. The resulting F2 kernels are expected to be 75% yellow, 18.75% pale yellow, 6.25% white if the tested yellow (Y1/Y1) line is Bn7/_, compared to 75% yellow, 25% white if the tested yellow (Y1/Y1) line is bn7/bn7. We confirmed that Bn7 is a single dominant gene by crossing an inbred line with suspected genotype Y1/Y1 Bn7/Bn7 to a genetic stock with known genotype y1/y1 bn7/bn7 and self-pollinating for one generation. The pollination yielded the genotypic classes as defined in Table 3, which failed to reject the hypothesis of a single dominant Bn7, with a Chi-square value of 1.49 based on 2 degrees of freedom.

TABLE 3

| Phenotype (Genotype | Observed | Expected |
|---|---|---|
| Y1, Genotype Bn7) | Kernels | Kernels |
| Yellow (Y1/_, _/_) | 223 | 229 |
| Pale Yellow (y1/y1, | 65 | 57 |
| Bn7/_) | | |
| White (y1/y1, bn7/bn7) | 17 | 19 |

Two F2 populations were then generated. The first population pop1, PL602C (y1y1bn7bn7) X PL713A (Bn7Bn7y1y1), contained 577 individuals. The second population pop2, WH120791228 (y1y1Bn7Bn7/PL602C (y1y1-bn7bn7), contained 750 individuals. The segregating progeny were phenotyped visually for all color classes described above. The parents of these populations were re-sequenced and SNPs were discovered as described in these Examples. A total of 87 KASP markers designed for the SNPs spanned between 115-135 CM on chr7 and genotyped using KASPAR genotyping technology as follows.

Primers were designed using the KRAKEN assay design software (Kbio). 1.5 μl of the 1:100 DNA dilution was used in the assay mix. 12 μl of 100 mM of each forward probe, and 30 μl of the common reverse primer was combined to make each assay. 13.6 ul of the assay mix was combined with 1000 μl of KASP Master Mix. A Meridian (Kbio) liquid handler dispensed 1.3 μl of the mix onto a 1536 plate containing ~6 ng of dried DNA. The plate was sealed with a Phusion laser sealer and thermocycled using a Kbio Hydrocycler with the following conditions: 95C for 15 min, 10 cycles of 95C for 20 sec, 61C stepped down to 55C for 1 min, 29 cycles of 95C for 20 sec, and 55C for 1 min. The excitation at wavelengths 485 (FAM) and 520 (VIC) was measured with a Pherastar plate reader. The values were normalized against ROX and plotted and scored on scatterplots utilizing the KRAKEN software.

A total of 70 recombinants were identified in the pop1 delimiting the Bn7 region to 120.9 to 121.5 (ZmChr7v2_156613548 to ZmChr7v2_156617084). A total of 16 recombinants were found in the pop2, delimiting the Bn7 region to 120.6-121.7 cM. In addition, fine-scale natural recombination at the inbred level helped place Bn7 between 121.2-121.59 (the region between C105495-00-Q001 (SEQ ID NO: 99) and C10549P-001-Q001 (SEQ ID NO: 104)), a region of ~0.39 cM. Finely mapped flanking markers of the Bn7 region from 120.97-121.59 cM were found from MaizeGDB using the gene model notation from Zm-B73-REFERENCE-GRAMENE-4.0. Markers used in assays to detect the Bn7 trait are listed as SEQ ID NOs: 99, 104 and 115-208 and are shown in Table 4.

TABLE 4
Markers for Bn7 region

| | | White | Yellow | Position of |
|---|---|---|---|---|
| SEQ ID | | SNP | SNP | SNP in SEQ |
| NO: | Marker | (bn7) | (Bn7) | ID NO: |
| 99 | C105495-001-Q001 | C | T | 51 |
| 104 | C10549P-001-Q001 | C | T | 51 |
| 115 | C10591M-001-K001 | T | C | 201 |
| 116 | C10591N-001-K001 | G | A | 201 |
| 117 | C10591P-001-K001 | T | C | 201 |
| 118 | C10591R-001-K001 | G | T | 201 |
| 119 | C10591T-001-K001 | A | C | 201 |
| 120 | Zmchr7v2_148557381 | T | A | 201 |
| 121 | C10591U-001-K001 | G | C | 201 |
| 122 | C10591V-001-K001 | G | C | 201 |
| 123 | C10591W-001-K001 | G | C | 201 |
| 124 | C10591X-001-K001 | A | C | 201 |
| 125 | C10591Y-001-K001 | G | A | 201 |
| 126 | C105920-001-K001 | G | A | 201 |
| 127 | C105921-001-K001 | G | C | 201 |
| 128 | ZmChr7v2_153902319 | G | C | 201 |
| 129 | ZmChr7v2_154022588 | G | A | 201 |
| 130 | ZmChr7v2_154156304 | A | G | 201 |
| 131 | ZmChr7v2_154330403 | A | T | 201 |
| 132 | ZmChr7v2_154330549 | T | C | 201 |
| 133 | ZmChr7v2_154330550 | G | C | 201 |
| 134 | ZmChr7v2_154330751 | C | G | 201 |
| 135 | ZmChr7v2_154331150 | T | C | 201 |
| 136 | ZmChr7v2_154331279 | T | G | 201 |
| 137 | ZmChr7v2_154331450 | C | T | 201 |
| 138 | ZmChr7v2_155005881 | A | G | 201 |
| 139 | ZmChr7v2_155012364 | A | G | 201 |
| 140 | ZmChr7v2_155053571 | C | T | 201 |
| 141 | ZmChr7v2_155241237 | G | A | 201 |
| 142 | ZmChr7v2_155241535 | T | C | 201 |
| 143 | ZmChr7v2_155288963 | G | T | 201 |
| 144 | ZmChr7v2_155289435 | G | A | 201 |
| 145 | ZmChr7v2_155351605 | C | T | 201 |
| 146 | ZmChr7v2_155410715 | T | C | 201 |
| 147 | ZmChr7v2_155413697 | G | C | 201 |
| 148 | ZmChr7v2_155442292 | G | C | 201 |
| 149 | C105922-001-K001 | G | A | 201 |
| 150 | ZmChr7v2_155506719 | C | T | 201 |
| 151 | ZmChr7v2_155508499 | C | G | 201 |
| 152 | ZmChr7v2_155518701 | C | A | 201 |
| 153 | ZmChr7v2_155518918 | T | C | 201 |
| 154 | ZmChr7v2_155519232 | G | A | 201 |
| 155 | ZmChr7v2_155519405 | G | C | 201 |
| 156 | ZmChr7v2_155520657 | T | C | 201 |
| 157 | ZmChr7v2_155521495 | C | T | 201 |
| 158 | ZmChr7v2_155521516 | G | A | 201 |
| 159 | ZmChr7v2_155521591 | C | T | 201 |
| 160 | ZmChr7v2_155524172 | G | C | 201 |
| 161 | ZmChr7v2_155539594 | G | T | 201 |
| 162 | ZmChr7v2_155540225 | G | A | 201 |
| 163 | ZmChr7v2_155586401 | A | C | 201 |
| 164 | ZmChr7v2_155663891 | A | G | 201 |
| 165 | ZmChr7v2_155666082 | G | A | 201 |
| 166 | ZmChr7v2_155675886 | T | G | 201 |
| 167 | ZmChr7v2_155675939 | C | G | 201 |
| 168 | ZmChr7v2_155676116 | G | A | 201 |
| 169 | ZmChr7v2_155676268 | G | C | 201 |
| 170 | ZmChr7v2_155680489 | C | G | 201 |
| 171 | ZmChr7v2_155768407 | T | A | 201 |
| 172 | C105923-001-K001 | T | C | 201 |
| 173 | ZmChr7v2_156404407 | A | G | 201 |
| 174 | ZmChr7v2_156404423 | A | G | 201 |
| 175 | ZmChr7v2_156413328 | T | G | 201 |
| 176 | ZmChr7v2_156413350 | G | A | 201 |
| 177 | ZmChr7v2_156413461 | C | G | 201 |
| 178 | ZmChr7v2_156413676 | C | G | 201 |
| 179 | ZmChr7v2_156413913 | A | C | 201 |
| 180 | ZmChr7v2_156413983 | A | G | 201 |
| 181 | ZmChr7v2_156414056 | G | T | 201 |
| 182 | ZmChr7v2_156414135 | T | G | 201 |
| 183 | ZmChr7v2_156414147 | C | A | 201 |
| 184 | ZmChr7v2_156415059 | AG | A | 201 |
| 185 | ZmChr7v2_156612669 | T | C | 201 |
| 186 | ZmChr7v2_156613548 | T | C | 201 |
| 187 | ZmChr7v2_156617084 | T | C | 201 |
| 188 | ZmChr7v2_157041859 | A | G | 201 |
| 189 | ZmChr7v2_157056914 | G | A | 201 |
| 190 | ZmChr7v2_157057145 | A | G | 201 |
| 191 | ZmChr7v2_157146769 | T | C | 201 |
| 192 | ZmChr7v2_157162320 | T | G | 201 |
| 193 | ZmChr7v2_157162696 | C | G | 201 |
| 194 | ZmChr7v2_157163045 | G | T | 201 |
| 195 | ZmChr7v2_157384566 | A | C | 201 |
| 196 | ZmChr7v2_157385181 | A | G | 201 |
| 197 | ZmChr7v2_157385619 | C | T | 201 |
| 198 | ZmChr7v2_157386399 | T | C | 201 |
| 199 | ZmChr7v2_157547698 | T | G | 201 |
| 200 | ZmChr7v2_157547871 | A | T | 201 |
| 201 | ZmChr7v2_157547933 | G | T | 201 |
| 202 | ZmChr7v2_157622874 | G | C | 201 |
| 203 | Zmchr7v2_157833774 | C | A | 201 |
| 204 | C105924-001-K001 | G | A | 201 |
| 205 | C105925-001-K001 | T | G | 201 |
| 206 | C105926-001-K001 | G | A | 201 |
| 207 | C105927-001-K001 | G | A | 201 |
| 208 | C105928-001-K001 | C | G | 201 |

TaqMan PCR assays were performed using KASPAR genotyping technology with KASP tagged primers used to amplify the sequences in Table 4. Each of the markers in Table 4 could be used to detect the bn7 trait, with markers found in SEQ ID NOs: 99, 104 and 148-186 being preferred. The results show that Bn7/bn7 is a single dominant/recessive locus that in the presence of double recessive type y1 improves white kernel color.

### Example 4. Detection of P1 Gene for Cob Color

The P1 gene is the major determinant of cob color and is located on chromosome 1 at 100.9 cM. White cobs are desirable by corn end users, creating a need to efficiently select for both improved white grain while also maintaining white cobs. Marker C0031KD-001 (SEQ ID NO: 209) is located upstream of the P1 gene, within the P1 enhancer, which affects gene expression. This marker locus anneals to the same locus as public marker PZE-101064790. An allele call of “C” at position 61 of SEQ ID NO: 209 signifies P1-ww, or white cob. The locus was confirmed using 637 white cobbed inbreds and 697 red cobbed inbreds.

The TaqMan assays were developed as follows: Primers were designed using the primer 3 software (found online at bioprod.phibred.com/primer3/cgi-bin/primer3_www.cgi). Probes were designed using Primer Express Software. 1.5 μl of the 1:100 DNA dilution was used in the assay mix. 18 μM of each probe, and 4 μM of each primer was combined to make each assay. 13.6 μl of the assay mix was combined with 1000 μl of KASP Master Mix. A Meridian (Kbio) liquid handler dispensed 1.3 μl of the mix onto a 1536 plate containing ~6 ng of dried DNA. The plate was sealed with a Phusion laser sealer and thermocycled using a Kbio Hydrocycler with the following conditions: 94 °C for 15 min, 40 cycles of 94 °C for 30 sec, 60C for 1 min. The excitation at wavelengths 485 (FAM) and 520 (VIC) was measured with a Pherastar plate reader. The values were normalized against ROX and plotted and scored on scatterplots utilizing the KRAKEN software.

### Example 5. Combining Marker-Assisted Selection with a Color Sorting Device

The use of marker preselection for white corn line development is aided using a color sorter to remove Y1/_genotypes (yellow phenotype) at any segregating generation stage. The strong phenotype of the Y1 locus facilitates efficient screening using kernel color. Selection for the white variant of Y1 (y1) is possible with visual selection, but is a time intensive process, increasing the expense of breeding white corn lines. Average personnel trained in basic seed sorting took an average sorting time of 34 minutes (+/−13 mins) to select about 200 appropriately colored kernels for a F2 grain sample. Using a VMEK Metrix color sorter to select the white kernels reduced sample time to less than one minute per sample. This selection from F2 populations was performed using many white x yellow crosses, to remove the yellow (Y1/_) genotypes as described in Example 1. Resulting kernels were entered into breeding, backcrossing trials using marker assisted selection for kernel color and other agronomic traits.

### Example 6: Marker-Assisted Backcrossing for White Kernel Color

Backcrossing yellow genetics into white backgrounds has historically required very large population numbers to successfully recover white progeny due to the recessive nature of the white corn traits, making the process expensive and unmanageable. Molecular markers for key white corn traits (Y1, P1-ww, Bn7) are used in selection and allow these alleles to be held in a heterozygous state, while the rest of the genome is selected for the yellow parent. Yellow Y1 kernels are optionally sorted and removed visually or using a color sorter according to Example 5. For example, the expected proportion of BC2S1 kernels that are fixed for three recessive traits is 0.024%, but implementing marker assisted selection for all three traits to maintain heterozygotes at BC1 and BC2 stages will improve the proportion of BC2S1 progeny with all three traits to 1.563%. The marker assays disclosed in Examples 1-3 are optionally used. In this case, marker assisted selection improves the acceptability ratio of kernels from 4096 unacceptable to 1 acceptable to 64:1 and reduces the necessary population size of each intermediate generation significantly, moving the scale of population development to a more cost and resource efficient point.

### Example 7: Enabling Efficient White Corn Doubled-Haploid (DH) Production

Currently the ability to efficiently produce doubled haploids (DHs) from crosses between white and yellow inbred lines for use in white corn breeding is inefficient due to the need to select for several recessive traits at one time and the interaction of Bn7, Wc1, and Y1 on the kernel phenotype and resulting lines. The addition of markers for key white corn traits such as P1-ww would enable efficient recovery of acceptable white lines through marker-assisted preselection of F2 kernels to enter into the doubled haploid process by reducing error associated with visual phenotyping. Using a visual selection process either requires selfing to F3 to observe line cob color and remove it before submitting, which is inefficient, or producing a proportion of red-cobbed lines which do not have the desired white cob phenotype and are unacceptable to the market. In addition to a need to select for cob color, error associated with phenotyping kernel color results in a large number of Bn7/Bn7 DHs, reducing the efficiency of DH production.

A white x yellow kernelled population is produced following a cross of white corn inbred line and a yellow corn inbred line, and marker preselection of F2 kernels for bn7 and y1 homozygotes, as well as P1 is performed, with selection from marker data being supplemented with visual or mechanical selection for y1 homozygotes (white kernels) such as described in Example 5. Double haploids are then produced. The selection process is expected to result in production of doubled haploids with uniformly acceptable white kernel and white cob color and containing a proportion of elite yellow corn genetics thus enabling increased genetic gain from the elite yellow parent for non-color traits in the DH lines which have white kernels and white cobs.

### Example 8: Enabling Efficient White Corn Single-Seed Descent Line Production

Currently the ability to efficiently produce single-seed descent lines from crosses between white and yellow inbred lines for use in white corn breeding is inefficient due to the need to select for several recessive traits at one time and the interaction of P1, Bn7, Wc1, and Y1 on the kernel phenotype and resulting lines. The addition of markers for key white corn traits would enable efficient recovery of acceptable white lines through marker-assisted preselection of kernels to enter into the single seed descent (SSD) process by reducing error associated with visual phenotyping. In the current state of this process, error associated with phenotyping kernel color results in many Bn7/Bn7 lines being selected along with Bn7/bn7 lines, reducing the efficiency of SSD production. In a white x yellow kernelled population, marker preselection of F2 kernels for bn7 homozygotes, and optionally one or more of P1, Bn7, Wc1, coupled with visual or mechanical selection for y1 homozygotes (white kernels) such as described in Example 5, which is less susceptible to error, would result in production of SSDs with uniformly acceptable kernel and cob color, thus enabling increased genetic gain for non-color traits.

The foregoing invention has been described in detail by way of illustration and example for purposes of clarity and understanding. As is readily apparent to one skilled in the art, the foregoing disclosures are only some of the methods and compositions that illustrate the embodiments of the foregoing invention. It will be apparent to those of ordinary skill in the art that variations, changes, modifications, and alterations may be applied to the compositions and/or methods described herein without departing from the true spirit, concept, and scope of the invention.

All publications, patents, and patent applications mentioned in the specification are incorporated by reference herein for the purpose cited to the same extent as if each was specifically and individually indicated to be incorporated by reference herein.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth. Unless expressly stated to the contrary, “or” is used as an inclusive term. For example, a condition A or B is satisfied by any one of the following: A is true (or present) and B is false (or not present), A is false (or not present) and B is true (or present), and both A and B are true (or present). No language in the specification should be construed as indicating that any non-claimed element is essential to the practice of the invention.

1-43. (canceled)

44. A method of producing a corn plant having non-yellow kernels and comprising a bn7 allele, the method comprising
(a) genotyping a plurality of corn plants or corn seeds having in their pedigree at least one non-yellow corn variety at a bn7 marker locus, the bn7 maker locus being within 5 CM of SEQ ID NO. 70 and associated with kernel color,
(b) selecting from the plurality of corn plants or corn seeds one or more corn plants or corn seeds comprising a bn7 allele contributing to whiter kernels; and
(c) crossing the selected corn plants or plants grown from the selected corn seeds with other plants comprising the bn7 allele or self-pollinating the selected plants or plants grown from the selected corn seeds to produce a plant having non-yellow kernels and comprising the bn7 allele.

45. The method of claim 44, wherein the bn7 marker locus is between and includes SEQ ID NO: 118 and SEQ ID NO: 206 or between and includes SEQ ID NO: 99 and SEQ ID NO: 104 and wherein the selected corn plant comprises one or more of SEQ ID NO: 43-98.

46. The method of claim 44, further comprising genotyping a y1 marker locus associated with at least one y1 recessive white allele for a polynucleotide encoding a polypeptide having at least 95% identity with SEQ ID NO: 110 and selecting from the plurality of corn plants or corn seeds in step (b) one or more corn plants or corn seeds comprising the bn7 white allele and the at least one y1 recessive white allele.

47. The method of claim 46, wherein the genotyping a y1 marker locus comprises detecting the zygosity state of the y1 locus, wherein the corn plants or corn seeds selected in step (b) comprise a heterozygous Y1/y1 allele and further wherein the corn plants or corn seeds selected in step (b) comprise:
i. SEQ ID NO: 5; or
ii. a C nucleotide at the position that corresponds to position 301 of SEQ ID NO: 19.

48. The method of claim 44, further comprising genotyping a Ccd1 marker locus associated with Wc1 encoding a polynucleotide encoding a polypeptide having at least 95% identity with SEQ ID NO: 112, wherein the Ccd1 marker locus comprises a plurality of copies Ccd1, and wherein selecting the plant in step (b) further comprises selecting a plant having at least three copies of Ccd1.

49. The method of claim 48, wherein the Ccd1 marker locus comprises SEQ ID NO: 29.

50. The method of claim 44, further comprising genotyping a P1 locus for white cob, wherein the plant selected is homozygous for bn7, homozygous for y1, comprises P1-ww for white cob/white pericarp, or a combination thereof.

51. The method of claim 50, wherein the plant selected is homozygous for bn7, y1 and has at least three copies of Ccd1.

52. The method of claim 44, wherein selecting the one or more corn plants or corn seeds in step (b) further comprises using a color sorter to separate the seeds on the basis of color.

53. The method of claim 44, wherein the plant produced in step (c) has white kernels, a white cob or a combination thereof.

54. A method of producing a maize plant having non-yellow kernels, the method comprising:
(a) genotyping a nucleic acid isolated from a maize plant for the presence of a marker located within a chromosomal interval defined by and comprising SEQ ID NO: 115 and SEQ ID NO: 208, the interval comprising a C corresponding to position 201 of SEQ ID NO: 157;
(b) selecting a first plant on the basis of the presence of the marker genotyped in (a); and
(c) crossing the first plant with a second plant to produce progeny seed and selecting a progeny seed or plant grown therefrom comprising the C corresponding to position 201 of SEQ ID NO: 157, thereby producing a plant having non-yellow kernels.

55. The method of claim 20, wherein the selected corn plant comprises a G at position corresponding to 201 of SEQ ID NO: 148, a G at position corresponding to 201 of SEQ ID NO: 149, a C at position corresponding to 201 of SEQ ID NO: 150, a C at position corresponding to 201 of SEQ ID NO: 151, a C at position corresponding to 201 of SEQ ID NO: 152, a T at position corresponding to 201 of SEQ ID NO: 153, a G at position corresponding to 201 of SEQ ID NO: 154, a G at position corresponding to 201 of SEQ ID NO: 155, a T at position corresponding to 201 of SEQ ID NO: 156, a G at position corresponding to 201 of SEQ ID NO: 158, a C at position corresponding to 201 of SEQ ID NO: 159, a G at position corresponding to 201 of SEQ ID NO: 160, a G at position corresponding to 201 of SEQ ID NO: 161, a G at position corresponding to 201 of SEQ ID NO: 162, an A at position corresponding to 201 of SEQ ID NO: 163, an A at position corresponding to 201 of SEQ ID NO: 164, a G at position corresponding to 201 of SEQ ID NO: 165, a T at position corresponding to 201 of SEQ ID NO: 166, a C at position corresponding to 201 of SEQ ID NO: 167, a G at position corresponding to 201 of SEQ ID NO: 168, a G at position corresponding to 201 of SEQ ID NO: 169, a C at position corresponding to 201 of SEQ ID NO: 170, a T at position corresponding to 201 of SEQ ID NO: 171, a T at position corresponding to 201 of SEQ ID NO: 172, an A at position corresponding to 201 of SEQ ID NO: 173, an A at position corresponding to 201 of SEQ ID NO: 174, a T at position corresponding to 201 of SEQ ID NO: 175, a G at position corresponding to 201 of SEQ ID NO: 176, a C at position corresponding to 201 of SEQ ID NO: 177, a C at position corresponding to 201 of SEQ ID NO: 178, an A at position corresponding to 201 of SEQ ID NO: 179, an A at position corresponding to 201 of SEQ ID NO: 180, a G at position corresponding to 201 of SEQ ID NO: 181, a T at position corresponding to 201 of SEQ ID NO: 182, a C at position corresponding to 201 of SEQ ID NO: 183, an AG at position corresponding to 201 of SEQ ID NO: 184, a T at position corresponding to 201 of SEQ ID NO: 185, a T at position corresponding to 201 of SEQ ID NO: 186, a C at position corresponding to position 51 of SEQ ID NO: 99, or a C at position corresponding to position 51 of SEQ ID NO: 104.

56. The method of claim 55, further comprising genotyping a y1 marker locus associated with at least one y1 recessive white allele and selecting from the plurality of corn plants or corn seeds in step (b) one or more corn plants or corn seeds comprising the C corresponding to position 201 of SEQ ID NO: 157 and the at least one y1 recessive white allele, wherein the genotyping a y1 marker locus comprises detecting the zygosity state of the y1 locus and wherein the corn plants or corn seeds selected in step (b) comprise a heterozygous Y1/y1 allele.

57. The method of claim 57, wherein the corn plants or corn seeds selected in step (b) comprise:
i. SEQ ID NO: 5; or
ii. a C nucleotide at the position that corresponds to position 301 of SEQ ID NO: 19.

58. The method of claim 57, further comprising genotyping a Ccd1 marker locus associated with Wc1 encoding a polynucleotide encoding a polypeptide having at least 95% identity with SEQ ID NO: 112, wherein the Ccd1 marker locus comprises SEQ ID NO:29 and a plurality of copies Ccd1, and wherein selecting the plant in step (b) further comprises selecting a plant having (i) at least three copies of Ccd1, (ii) a P1 locus for white cob, or (iii) a combination of (i) and (ii).

59. The method of claim 55 wherein the plant selected is homozygous for bn7, homozygous for y1, comprises P1-ww for white cob/white pericarp, has at least three copies of Ccd1, or a combination thereof.

60. The method of any one of claim 55, wherein selecting the one or more corn plants or corn seeds in step (b) further comprises using a color sorter to separate the seeds on the basis of color.

61. The method of claim 55, wherein the plant produced in step (c) has white kernels, a white cob, or a combination thereof.

62. A method for introgressing one or more alleles contributing to kernel whiteness into an elite yellow corn plant, the method comprising
(a) genotyping at a bn7 marker locus a plurality of seeds from a population of seeds, the population derived from a cross of an elite yellow corn plant with a non-yellow corn plant, the bn7 marker locus being within 5 CM of SEQ ID NO. 70 and associated with kernel color;
(b) selecting progeny plants comprising at least one of: a C corresponding to position 51 of SEQ ID NO: 99, a C corresponding to position 51 of SEQ ID NO: 104, a G at position corresponding to 201 of SEQ ID NO: 148, a G at position corresponding to 201 of SEQ ID NO: 149, a C at position corresponding to 201 of SEQ ID NO: 150, a C at position corresponding to 201 of SEQ ID NO: 151, a C at position corresponding to 201 of SEQ ID NO: 152, a T at position corresponding to 201 of SEQ ID NO: 153, a G at position corresponding to 201 of SEQ ID NO: 154, a G at position corresponding to 201 of SEQ ID NO: 155, a T at position corresponding to 201 of SEQ ID NO: 156, a C at position corresponding to 201 of SEQ ID NO: 157, a G at position corresponding to 201 of SEQ ID NO: 158, a C at position corresponding to 201 of SEQ ID NO: 159, a G at position corresponding to 201 of SEQ ID NO: 160, a G at position corresponding to 201 of SEQ ID NO: 161, a G at position corresponding to 201 of SEQ ID NO: 162, an A at position corresponding to 201 of SEQ ID NO: 163, an A at position corresponding to 201 of SEQ ID NO: 164, a G at position corresponding to 201 of SEQ ID NO: 165, a T at position corresponding to 201 of SEQ ID NO: 166, a C at position corresponding to 201 of SEQ ID NO: 167, a G at position corresponding to 201 of SEQ ID NO: 168, a G at position corresponding to 201 of SEQ ID NO: 169, a C at position corresponding to 201 of SEQ ID NO: 170, a T at position corresponding to 201 of SEQ ID NO: 171, a T at position corresponding to 201 of SEQ ID NO: 172, an A at position corresponding to 201 of SEQ ID NO: 173, an A at position corresponding to 201 of SEQ ID NO: 174, a T at position corresponding to 201 of SEQ ID NO: 175, a G at position corresponding to 201 of SEQ ID NO: 176, a C at position corresponding to 201 of SEQ ID NO: 177, a C at position corresponding to 201 of SEQ ID NO: 178, an A at position corresponding to 201 of SEQ ID NO: 179, an A at position corresponding to 201 of SEQ ID NO: 180, a G at position corresponding to 201 of SEQ ID NO: 181, a T at position corresponding to 201 of SEQ ID NO: 182, a C at position corresponding to 201 of SEQ ID NO: 183, an AG at position corresponding to 201 of SEQ ID NO: 184, a T at position corresponding to 201 of SEQ ID NO: 185, or a T at position corresponding to 201 of SEQ ID NO: 186; and
(c) performing a plant breeding step selected from (i) backcrossing the selected progeny plants to the elite yellow corn plant or a different elite yellow corn plant and further selecting further progeny plants for further traits selected from bn7, y1, at least three Ccd1 copies and any combination thereof; and (ii) further selecting further progeny plants for further traits selected from bn7, y1, at least three Ccd1 copies, P1 and any combination thereof and producing doubled haploids from the further selected progeny plants,
thereby producing a corn plant which comprises white kernels and otherwise comprises substantially all of the loci of the elite yellow corn plant.

63. The method of claim 62, wherein the genotyping in step (a) further comprises genotyping for y1, P1, Ccd1 or any combination thereof, and wherein selecting progeny plants in step (b) comprises selecting for the presence of a (i) y1, (ii) P1-ww, (iii) at least three copies of Ccd1, or (iv) any combination thereof.

64. A method of producing a maize plant having white kernels, the method comprising:
(a) genotyping a nucleic acid isolated from a population of maize plants for the presence of a marker located within a chromosomal interval defined by and comprising SEQ ID NO: 148 and SEQ ID NO: 186 the interval comprising a C allele at a position corresponding to position 201 of SEQ ID NO: 157;
(b) selecting a first plant on the basis of the presence of the marker genotyped in (a); and
(c) crossing the first plant with a second plant to produce progeny seed and selecting a progeny seed or plant grown therefrom comprising the C allele, thereby producing a plant having white kernels.


SEQUENCE LISTING (209 sequences)

SEQ ID NO: 1 - DNA, 627 bp, synthetic construct
  source 1..627: mol_type = other DNA
ttcttctctccacgtctctccccccttctttctccagascgagcgtacgtatgctacacacagcaacagcacaacagtactagttccaccacaagaagatgcccaatgcmaagaaataacccatgcttcttgtcgacgatccagccgcactagagatggccaaacgggccggcccggcccggcccggcccgggcccggtgaagcccggccaaaaccggnccgggcctgctgagccagcgggcttaagtttctgtccaagcccggcccgcagcgggcctaaacaggccgggccggcccgtttagcacgaaaaaacgggccaaaaagcgggctaaacgggccggtaagcacgttttagtgtaaaaaaaacgggcttaacgggcttagaggtaaacgggccgtgccgggctagcccgccgtgcctagtttcctgtccaagcccgcccgcttattctaccgtgccgggctcggaccggncccaaaaagcgggcttcgtgccgggctcacgggcctcgtgctttttggccatctatgagccgcacacttagcatacatacgcaagaagaggagaggccggaggtgcgcgtgctccttgctgctgttctgctgactggtctcaccatctcatc

SEQ ID NO: 2 - DNA, 13 bp, synthetic construct
  source 1..13: mol_type = other DNA
cagcgggcctaaa

SEQ ID NO: 3 - DNA, 20 bp, synthetic construct
  source 1..20: mol_type = other DNA
gcgggcttaagtttctgtcc

SEQ ID NO: 4 - DNA, 24 bp, synthetic construct
  source 1..24: mol_type = other DNA
ttttacactaaaacgtgcttaccg

SEQ ID NO: 5 - DNA, 237 bp, synthetic construct
  source 1..237: mol_type = other DNA
ttcttctctccacgtctctccccccttctttctccagascgagcgtacgtatgctacacacagcaacagcacaacagtactagttccaccacaagaagatgcccaatgcmaagaaataacccatgcttcttgtcgacgatccagccgcacacttagcatacatacgcaagaagaggagaggccggaggtgcgcgtgctccttgctgctgttctgctgactggtctcaccatctcatc

SEQ ID NO: 6 - DNA, 13 bp, synthetic construct
  source 1..13: mol_type = other DNA
ccgcacacttagc

SEQ ID NO: 7 - DNA, 20 bp, synthetic construct
  source 1..20: mol_type = other DNA
cacacagcaacagcacaaca

SEQ ID NO: 8 - DNA, 20 bp, synthetic construct
  source 1..20: mol_type = other DNA
gatggtgagaccagtcagca

SEQ ID NO: 9 - DNA, 401 bp, synthetic construct
  source 1..401: mol_type = other DNA
ccgcagcggtcgtaggcttccttgagcccgttgcgtggcatgtccatgtcctggggcctggcgtcgaggaccggcgtgcgcagctggcgtttgagcaatgcggcctgcttgagcacgacgtcgtagaccttctgctcggacgagacgacggcctctcccgccgggttgacggcgaggctggagtakacggcgggggagggrcggccrrcstccsacgggtggaggccaakgagcgagcagggcatccaccggcgygccgccggcctcttmgtcttgagcagggtggagcactggagagtcccctggtggctgatgctgtcggcggcggagagccccggcgacgctgctcgtacgagtatgatggccatatatttgctatcttatcctaaagatggtggtgg

SEQ ID NO: 10 - DNA, 12 bp, synthetic construct
  source 1..12: mol_type = other DNA
ggagggacggcc

SEQ ID NO: 11 - DNA, 12 bp, synthetic construct
  source 1..12: mol_type = other DNA
ggaggggcggcc

SEQ ID NO: 12 - DNA, 20 bp, synthetic construct
  source 1..20: mol_type = other DNA
cgacgtcgtagaccttctgc

SEQ ID NO: 13 - DNA, 19 bp, synthetic construct
  source 1..19: mol_type = other DNA
gctccaccctgctcaagac

SEQ ID NO: 14 - DNA, 401 bp, synthetic construct
  source 1..401: mol_type = other DNA
ggcctggcgtcgaggaccggcgtgcgcagctggcgtttgagcaatgcggcctgcttgagcacgacgtcgtagaccttctgctcggacgagacgacggcctctcccgccgggttgacggcgaggctggagtakacggcgggggagggrcggccrrcstccsacgggtggaggccaakgagcgagcagggcatccaccggcgygccgccggcctcttmgtcttgagcagggtggagcactggagagtcccctggtggctgatgctgtcggcggcggagagccccggcgacgctgctcgtacgagtatgatggccatatatttgctatcttatcctaaagatggtggtggtggtggtggtggtgggatgagatggtgagaccagtcagcagaacagcaaggagc

SEQ ID NO: 15 - DNA, 12 bp, synthetic construct
  source 1..12: mol_type = other DNA
caccggcgtgcc

SEQ ID NO: 16 - DNA, 12 bp, synthetic construct
  source 1..12: mol_type = other DNA
accggcgcgccg

SEQ ID NO: 17 - DNA, 20 bp, synthetic construct
  source 1..20: mol_type = other DNA
cgacgtcgtagaccttctgc

SEQ ID NO: 18 - DNA, 19 bp, synthetic construct
  source 1..19: mol_type = other DNA
gctccaccctgctcaagac

SEQ ID NO: 19 - DNA, 601 bp, synthetic construct
  source 1..601: mol_type = other DNA
gtgcttttcttgctctgccacccaaattgttcgttccctccccctcgctagcagctcacgcccaccaagtgacgacgagagggagacgggacacgcactttctgttgccgtgctctcacccaaacttgcaaccacaatatatatataagccaccggattgataatatggaaatggggaataaatgaaaaggtgccatgcatcatgcatggatatgcgaatgataatgcttgctagccgaatgttaggtacgggacaggactgaagctgatcgagcacttgcatacagtaatacctaaaccyactgtaaagcaaaattgccgagagaagaagcaggcaagcagcagtgtcaaacaactgtaagaaagcaaataaagcgactaaaacaaygactgcatgcatgtgctgctratccatccatccatcggcatcatcacgtaaaagaaaagaaaaggcagagccgtaaacggtaatttattatacacataataaatagctaataagctactccttcttgtcctaagttaaaattaaattcgttttagttaattaatagattcatataatacttratgttgatctatgtgttttatatgcgtctag

SEQ ID NO: 20 - DNA, 19 bp, synthetic construct
  source 1..19: mol_type = other DNA
ctaaacctactgtaaagca

SEQ ID NO: 21 - DNA, 18 bp, synthetic construct
  source 1..18: mol_type = other DNA
cctaaacccactgtaaag

SEQ ID NO: 22 - DNA, 20 bp, synthetic construct
  source 1..20: mol_type = other DNA
gtacgggacaggactgaagc

SEQ ID NO: 23 - DNA, 20 bp, synthetic construct
  source 1..20: mol_type = other DNA
ctgcttgcctgcttcttctc

SEQ ID NO: 24 - DNA, 314 bp, synthetic construct
  source 1..314: mol_type = other DNA
agaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaamtgagctctcacaggctagcagatggccagtaagtccactcaacttcacattkcccacccagtatagcacagcatcctcacttcyttttctttgttaccattgcaggtatgggcttccctgttgttgtacaggcagatcctggatgagatcgaagcc

SEQ ID NO: 25 - DNA, 17 bp, synthetic construct
  source 1..17: mol_type = other DNA
ctgtgagagctcattta

SEQ ID NO: 26 - DNA, 18 bp, synthetic construct
  source 1..18: mol_type = other DNA
cctgtgagagctcagtta

SEQ ID NO: 27 - DNA, 22 bp, synthetic construct
  source 1..22: mol_type = other DNA
ttcatgaagaggcagatcaaga

SEQ ID NO: 28 - DNA, 20 bp, synthetic construct
  source 1..20: mol_type = other DNA
ggacttactggccatctgct

SEQ ID NO: 29 - DNA, 438 bp, synthetic construct
  source 1..438: mol_type = other DNA
aggtagaatttagtttatgcaagaaactctataaagaagatatttttagtatcgattcacacgttgcttcacatgttctatcctgacgactctactagatataatgctacttgtgagtagaaagaagattcttttattatattttaagtttgtaagagcctttagaaccggttgatggtaaaagctggcagtaaattaagggggtgtttaaatgcactagaactaatagttatttagctaaagattgttagtgaaattagatagctaaccaataactatctaactattaactaatttatcaaaaatagctaatagctgaaatattagccagggtgttttgatgtctcaactaattttagtcactaactattagttctaatacattcaaacacccctaagagccatttggtactggtttttgccatcaaacaatactaa

SEQ ID NO: 30 - DNA, 22 bp, synthetic construct
  source 1..22: mol_type = other DNA
aaatgcactagaactaatagtt

SEQ ID NO: 31 - DNA, 25 bp, synthetic construct
  source 1..25: mol_type = other DNA
ggtaaaagctggcagtaaattaagg

SEQ ID NO: 32 - DNA, 21 bp, synthetic construct
  source 1..21: mol_type = other DNA
gacatcaaaacaccctggcta

SEQ ID NO: 33 - DNA, 217 bp, Zea mays
  source 1..217: mol_type = other DNA
agagcaacatgtgtgatytgctcaggatcaacacygaccgcggtgtgatgattgscgatggcaagtcgcggttttcaatcaatgggaagcctatctaccactttgttgggacttccaccttcagcgagtacacygtcatgcatgtsggttgtgttgcaaagatcaaycctcaggctccccttgataaagtttgcgtccttagctgtggtatttctac

SEQ ID NO: 34 - DNA, 19 bp, synthetic construct
  source 1..19: mol_type = other DNA
tgggaagcctatctaccac

SEQ ID NO: 35 - DNA, 20 bp, synthetic construct
  source 1..20: mol_type = other DNA
caagtcgcggttttcaatca

SEQ ID NO: 36 - DNA, 21 bp, synthetic construct
  source 1..21: mol_type = other DNA
tgaaggtggaagtcccaacaa

SEQ ID NO: 37 - DNA, 27 bp, synthetic construct
  source 1..27: mol_type = other DNA
ctcattacctgccgccttgagaatcca

SEQ ID NO: 38 - DNA, 22 bp, synthetic construct
  source 1..22: mol_type = other DNA
gggaagagggtgatgaagttgt

SEQ ID NO: 39 - DNA, 24 bp, synthetic construct
  source 1..24: mol_type = other DNA
tgatatccattcaccttgtccaaa

SEQ ID NO: 40 - DNA, 19 bp, synthetic construct
  source 1..19: mol_type = other DNA
tgggaagcctatctaccac

SEQ ID NO: 41 - DNA, 20 bp, synthetic construct
  source 1..20: mol_type = other DNA
caagtcgcggttttcaatca

SEQ ID NO: 42 - DNA, 21 bp, synthetic construct
  source 1..21: mol_type = other DNA
tgaaggtggaagtcccaacaa

SEQ ID NO: 43 - DNA, 597 bp, Zea mays
  source 1..597: mol_type = genomic DNA
atgcagaagctagtccagcggtccttgaaggaggaagagaagctgcagcatatgcttgcgaacttaccttctggaatgcgcaagaatgattttgctaatcagcacgaacagagctcatcaaggatgctgcctcattttaactcttcttttactgaagctaatgaatgtgaactgaagattaaggatgaaccagttgcagctcctaggaagggaagagcacctgcacctcgttggtatatctcaaccgaggagcttgattcactctcatcgtacatgaggggaagactgactttggaaaaggttaatatcgcaataaatgaagtggcttcatatgcagatgccaatgctcatcttgttacatgccctaagaaaaagttgtcagaagatacatgggaaaaagctttggaactaagggatattgcagcaagtggtgcagtaaagggcaaccatttcttcctggaagcagatataaaaggacctgggttaaaacttgacaatactggcaaagctattcttaccgttcttcgccaccttgggcgcttccatgaagttcggattgggcaccatcgggtctttatactgtcaaagcaggcatga

SEQ ID NO: 44 - DNA, 1245 bp, Zea mays
  source 1..1245: mol_type = genomic DNA
atggcgaccagcaacggctcaggggtcgcgcagggggaggaggggaagacgccaccgcctgcggggtcgatcggcgggtacgagtccctgcacaggctcctggaggccaacctttcccccgatctattccaggaagcaagtcgattgctgctgggactgaattgtgcacgacctcttgaggctatctcactgcctgacgccacaaccactcttgcggaaacacacagttttgatttgcaggcattccgttttagtgcagacaaagagtttctgaggcaatcaagagttgttcgagtaggtctaattcagaactcaattgctgttcccactacttgtcactttgctgaccaaaagaaggcaatcatggacaaaataaaacctctaattgatgcagccggcgcttctggtgtcaatattatgtgcttacaagaagcctggacaatgccttttgccttctgcacacgcgagaaaagatggtgtgaatttgctgaaccagttgatggagaatctactcaattccttcaggaacttgcacagaagtataacatggtaatagtgagcccaatccttgaaagggatataaaccatggggagactatttggaatactgctgttgttattggaaacaacggcaacataattggcattcatcgaaagaatcacattccaagagttggtgatttcaatgagagcacatactacatggagggtaacactgggcatccagtgtttgaaacagcttatggcaaaataggagtgaacatttgttatggaagacatcatccccttaattggcttgcgtttggtctcaatggagctgaaatagtgttcaacccatctgcgactgttggtgaactgagtgaaccaatgtggcccattgaggcaaggaacgccgcaattgcaaacagctacttcgttggatcaattaatcgggttggcacagaagtgttccctaatcctttcacatccggtgatgggaaaccgcaacacgcagactttggccatttctatggatctagccatttctcagcacctgatgcctcctgcaccccatctctatcacgctaccgagatggcttgatcatctccgatatggacctcaacctatgccgccagatcaaagacaagtgggccttccgcatgactgctcgctatgagatgtatgcatccttgctctcagagtacttgaagccagatttcaaacctcaagtcattgttgatcccttgattaataagaaggcataa

SEQ ID NO: 45 - DNA, 1650 bp, Zea mays
  source 1..1650: mol_type = genomic DNA
atgtcggcggccgccgccgcccggctcctccgtttggccccgcgccggctccaggttccaaaggcctccccgctcgccgcgatttcgttcccgctcccgcgtacgacacctctctccgctgcctcggggcggcggcagcgcttctgcgccgcggcgcaggcccccgcccccgcatacgcggcgacagggacggcgggcgaggccgtgggggagttcaggaagcggctgcgggtggcggacgtgaagggcggggaggacgagggcgctgcctgggtgggcaaggagctcgccgtgcgcgggtgggtgcgcacctgccgcgcccagaggaccgtcaccttcgtcgaggtcaatgatggctcttgcttgtccaatatgcaatgtgtgttgactcctgaaacagaaggctatgaccagatagactctgtcactactggagcatctgtactagttgagggagttgttgcaagcagccaaggcggtaagcaaaaagtggagttgaaggtttcaaagatcactgtgattggtaagagtgaccccacatcttttcctatacagaagaaacgggcatcaagagagttccttagaactgtagcccatctccgtcctcggacaaacacttttggtgcagtggcaagagtgagaaatgctctggcatacgcaactcataaattctttcaagacaatggatttgtttgggtttcaagcccaattattactgcctcagattgtgaaggagctggggagcagttttacgtgactactttgctttcgaatagcaccgaaggtggttccctactcaaagatattcctgcgaccaaggatggaagggttgattggtcacaggatttcttctgcaaaccagcatttctgacagtatctggacagctcaatggtgaaacatatgcttcagctctatctgatatttacacatttggtccaacgtttagggctgaaaattcaaacactgcaaggcatctggctgaattttggatgattgaacctgagcttgcctttgcggatctaaatgatgacatggcttgtgcaactgcatatctccagtatgtagtaaagtatattctagagaactgcaaagaggatatggatttcttcaatacatgggttgagaaaggcatcatagatcgattaaatgatgtagtagagaagaactttgttcacttgtcttacactgatgctgtcgagctactacttgggtccaagaagaaatttgagtttccggtaaagtggggtttggatcttcaaagtgagcacgagcgatatatcacagaagttgcctttgatgggcgccctgtaataatcagagattacccaaaggaaatcaaagctttctatatgagagaaaatgatgatgggaagacagttgctgcaatggatttgttggttcctcgggttggtgagcttattgggggaagccagagggaagagcgtcttgattaccttgaagcccgactagacgagcaaaatctgaacaaggagagctactggtggtatctggacctacggcgatatggatcagttccccacgccggtttcggtcttggatttgagcggcttgtacagtttgctactggaatagacaacatcagagacgccatcccatttccccgagttcctggctcagccgagttttag

SEQ ID NO: 46 - DNA, 615 bp, Zea mays
  source 1..615: mol_type = genomic DNA
atgggggcgtacaagtatgtctcggagttatggaggaggaagcaatccgacgtgatgcggttcgtgcagcgcgtgcgctgctgggagtacaggcagcagccggccatcgtccgcctcacaaggcccacccgccccgacaaggcccgccgcctcggctacaaggccaagcaggggtatgttgtttaccgtgtccgtgtcaagcgtggcggcaggaagaggcctgtgccgaagggtattgtctatggcaagcccaagcaccagggtatcacccagctcaagttccagaggaacaagaggtctgttgctgaggagagggctggacgaaagcttggtggactcagggttctcaactcctactgggtgaatgaggatagcacctacaagtacttcgagatcatcctggttgatgtggctcacaccgccatccgcaacgacccaagaatcaactggctatgcaatcctgtgcacaagcaccgtgagctccgtggtctcacctctgcaggcaagaagttccgtggtctgcgtggcaagggccacactcaccacaagaacaggccgtccaggagggccacctggaagcgcaaccagaccctttccctccgccgctaccgttga

SEQ ID NO: 47 - DNA, 510 bp, Zea mays
  source 1..510: mol_type = genomic DNA
atggctacatatacaacctcaaactcaactgaagagtattacaccaatagtggatgtgatggggattgtcttcagccagttcacgtcattcttggatttgattcagttttccttgggaggtccgacattaagtgtgtccagctgaatggagccatgaacatcaccgaaaagggaagagccatagacatcttcacccatgatgcagactacagggtcttcctgatgagcttaaaagctggaggagtcgcgctgaatctcactgtagcatctcatgtgttcctgatggacccttggtggaatccagcggtcgagaaccaagcgcaggaccgtatccaccgaattgggcagttcaagccaatcaagagcactaggtttgtgataaaggacacggttgaagagcgcatactgcaactgcaacagaagaagcagctggtgtttaaaggcaccatgggggactcgccggatgccatgtcgaagcttacagaggcggacctcaagtttatgttctag

SEQ ID NO: 48 - DNA, 369 bp, Zea mays
  source 1..369: mol_type = genomic DNA
atgtctgtaagtccagccaaatttgagcagaaaggtgatgttttcgtttcaaagaaaactgacaagcaaaagaaaaggaagataaagaaggttgaggacaaaatgcttggatggggtgggcatgacgataagaagctgatgattcctgctacagtaattttaaggcacatgttcactcctgttgagctgagagctgatgaagagcttctttttgagcttgaggctgatgttagagaggaatgcatcaagtttggtctggtggataatgtcaaggtttgtgaaaatcatccacaaggtgtcatattggtaagattcaaagacaggaaagatggtgccaaatgcatagagaagatgagcggcagatggtaa

SEQ ID NO: 49 - DNA, 615 bp, Zea mays
  source 1..615: mol_type = genomic DNA
atgggggcgtacaagtacgtctcggagttatggagaaggaagcaatccgacgtgatgcggttcgttcaacgcgtgcgctgttgggagtacaggcagcagccggccatcgtccgcctcacaaggcccacccgccccgacaaggcccgccgcctcggcttcaaggccaagcaggggtatgttgtttaccgcgtccgtgtgaggcgtggtggcaggaagaggcctgtgccgaagggtattgtctatggcaagcccaagcaccagggtatcacccagctcaagttccagaggaacaagagatctgttgctgaggaacgggctggacgaaagcttggtggactcagggttctcaactcctactgggtgaatgaggatagcacctacaagtactttgagatcatcctggttgatgtggcccacactgccatccgcaacgacccaagaatcaactggctatgcaatcctgtgcacaagcatcgtgagctccgtggtctcacctctgcaggcaagaagttccgtggcctgcgtggcaagggccacactcaccacaagaacaggccgtcgaggagggccacctggaagcgcaaccagaccctgtccctccgccgctaccgttga

SEQ ID NO: 50 - DNA, 786 bp, Zea mays
  source 1..786: mol_type = genomic DNA
atgccgctggctgagtcgcccccgtggcgccgcaaggccacagatttcttctccacgtccagtgtcaagctgaagcaggcaggccaatcggccggggataatatagttgatgttgctgggaaggttgggtccgtggtgaagagtcggtgggctgtcttccaagaggctaggcagcagcagcagcagcagcaacgtccgccgcatgagacagtgcaagagcgtatcatcactgctgctgcctccactggtttgcttttcaggaaaggcatttcagagacaaaggagaaggttgcagtgggaaaggtcaaagttgaagaggctgctaaaaaaactgcagataaaagcaagagtatcttgaacaatattgaacgctggcagaagggagtcgcaagcactgatgtgtttggtgttcctattgaagctactgtacaacgagagcaatctgatagagcggtgcccctggtgcttgtgagatgtgcggactacctggttatatcaggtttgaataatgagtacttattcaaatctgaaggtgacaaaaaagttcttcagcagttagtttctctttacaatgaagactcgggtgcatctttacttgaaggtgtaaatcctattgatgtaggtgcactggtgaagtgctaccttgccagtatcccggagccacttactacattttcgctttatgatgagcttcgagctgcgaggcgttgctgctgcgagtcagccgtaaatcatcacttaacaagatggactcccgcagccttgctgtggaatttgcgcctttga

SEQ ID NO: 51 - DNA, 1725 bp, Zea mays
  source 1..1725: mol_type = genomic DNA
atgctgctacgcgccgcgctccacctcaaccgccttttctccactgacgccgccctcgcacccccgaagctgcggaacctgccctaccgtctccgtcacgtcgccgtccccgccgcgcgcgctgtcgtctccgagtacctccactcgacgcgctgcctcccgtcctcccacgccgactccatcgccacgcactccccgcgctccctcttttccttcctcgccgcgctccccaccgtgccgtgctccctccccaccgcggagtttcccgccctccttcgccgccacctcaacttccacccgctcaatgagctccccttcttcctcgaatcaatcggcgggcccaccgcggcgcccccctgcttcaactccatcttcctttccgaccatccctccctgctcggcgccgtcgccgcgctcgcgcacttcggattcccgtggtcacgcctcggcctcctctaccctggcgtcctcctcgacatgcccccggacctcatctccacgcgcctctccgttctcgaggcccgcctccacgggcttcctcgcgccgccatcatcgccgcctgcctcaccttcccatcacttctcgagggggatctatccgattatgacctactcattaaggatatcgccaccacgttcataggattgggaccggatttgagggccagcaatgacattgacgcgttctcggcggtgtgccggaggatgcggatgttttatgatgccgggacagagtttggtagcattgggggactcgtcggaggcagccagagggtgtttcttgagctcggggagaagaggattgctgagaggttatggttcttcaaagagctggggatggagggcaaggagatgggaaggtttttgctgagcaatgccaggatttttgatcttgatttctctgatgtggtgatctcagtgccaagatatctgctgaatgttggattggcagaggatgaggttgatgctgcggtggggaagcacccatatgttgttgggaagaaccagctggagaaccttgccagggtgctgcgggcaatggagctggagcatcggttcctggagaagatattggcaggtggggagagtttgcgctatttatcaccagaatttgttttggaggatgatagctatgatgccgaggtcgaaagagctttcttggatgggatggctaaggtcatggttgacaggaaagcacaatttgtagacaagaagctggagttcttgaaaagtgttgggtatggtgagaatgagataaccaccaaggtaattccagtcattaacagcaccaaggatttgctgttggagcggtttgactacctcctggaaagaggagtggagtataagatactatgtcggatcttgcgcgtttttccgaaggtgctgaaccagagtaaggatatgcttaatgagaagttgaactacctcacagaggagctgggctactccttggagtacctgggctgcttccctgcatttctgtgctttgatttggagaacagggtcaaacctaggtatacaatgctttggtggctccaagagcatggcttgcttagaaaaaaattagcccctgcaacagtgcttgctaactcagagaagaggttcattagtaccctttaccttgtgcatcctgcagttccaaagttgtggcttgagtgtttttcttcaaggatacatatggactattatctcaagaacatcaatcagcatccagacaacatataa

SEQ ID NO: 52 - DNA, 699 bp, Zea mays
  source 1..699: mol_type = genomic DNA
atggtgatgcatctgtattctcgctcctcttccgccggcatgcgaagcattcgccgggagctccagcgcaggaggtccaagcccttggcgccgacagggtcggtcgctaagatgccgtccgcgtcgcgtcacgcttcgagcggcggcggcgtccgggatccatgtcctcgtccaccgcatcgagcggccccgtcttcgacaatccgccatttccaaggcaccacagtcaccgacgcgtcacaacaccgtcggcctcgcgcttcgtcgcgctgcacccccgcctctcctccgccacctcggtctgccccatctcctggcgtccccgcgcatctgaagcctgggacggtggttggtgtccgcacgaggactacaacgctgaagacggggcaggttctcgtgctgtggctcaaagccacgatcgtgtcgtcgtctgccgacggatccggctacggtgttgtctacgacggcaactggccgcaaggcgatcccaagagcacggtctacgtcgcgccgcaccaagtcaggctattcagtccgtctccatcgccgacgactccaccgccgtccctgcctccacccaccgccactgccgccgcgactactaggaagaaggacaggccgaggccaaccacggcagggaagagcctgcgcctcatgcgcagcctctttccggagatggagttccccacccgggcttga

SEQ ID NO: 53 - DNA, 576 bp, Zea mays
  source 1..576: mol_type = genomic DNA
atggcggcaaccgcgcacctcaccggtgtcctcggcgtgtcgtcgccgcctctcgctccgtcccactgcagctgcagcggcgccaagaagcagagctgctcgctgcgtcaggggcggcagcagaggaggcgcctccgcgtcgcccgcgccgtcgaggtggacgcgccttccgccggcgcgccggagccggaggaggcggaggagccgtccgtcgacttcgcgttcgtcagcccgcggctgctgccggacgggacgccggacgtgcactaccggacggcgcgcggcgggcagaagctcagggacatcatgctcgacggctacatcgacctctacgggccctacgataaggttcttctcaactgctcagggggcggcgtgtgcgggacctgcatcgtcgaggtggttgaaggcaaggaaatgctgtccccgaaaactgatgtggaaaaggagttgctcaaaaggaaacccaaaacgtggagattggcgtgccaggcaacggtgggcaacgcagattcgactggacagatgatcattcaacagttgccggagtggaagatacacgagtgggacaagtag

SEQ ID NO: 54 - DNA, 2727 bp, Zea mays
  source 1..2727: mol_type = genomic DNA
atggcgggtggcgccggcgggcgggacccgctcgtcgcctccgagatccacggcttcctcacctgcgcaggtcgccccgccccgcccccgcacgagggcggccttttctccctccgttttctcttcttctcctttggtggtactgttgttctatatgatcgtaaagttgtccggaacttccgtaaagatggccataactggaagaaaaagaaagatggcaagactgtccaagaagcccatgaaaaactaaagattggtaatgaagaaaaggttcatgtgtattatgctcgaggagaggatgatccaaatttctttcgcagatgctactggctacttgacaaagagttggagcgcattgtcctcgtgcattatcgtcaaacatctgaggaaagtgccttaccaccttcgcatgtagaagctgaagttgcagaagtgcctcggatcaatatgattcattacacctcctcaacagattcagcctctgctcatactgagctatcttcttctgctgctgctgcaccagaagatatcaactcaaatggtggtggggcggtttctagtgaaaccgacaatcaaggttctagcctggaatcattttgggtagatctattggagtcatctatgaaaaatgatactcctgttgatgcttctgcttgcggtggatctttggtatcaaatcagcagactaataatgggatgggagattctggaaataatatcttgtatattaatgccacaagtaatgctatcttttctccaccaactaatgtggtttcagaagcttatgctaatcctggccttagtcaagtatcagagagttactttggatctctgaaagatcaagcaaaccatgctccatctcttttaacatcagatctggattcacaatcaaagcaacatacaaattctttgatgaaaactccggtgtctgataacatgccaaatgatgtacctgctaggcagaacagtcttgggttatggaaatacttggatgatgatattagtttagatgataatccaagctcgggcatactccccactgaacaggtgactggcgaaataccctttcagatcactgagatatcctcagaatgggcttattgtactgaggacacaaagaactacccaaataattgcttacttcctggtttggtggatgtggaggtccttgtggttggatgtttccatgagaactacagacatctggctgggaccaatctcttctgtgtcattggtgaccagtgtgttgatgcaaatattgttcaaactggcgtttaccgtttcattgctagaccacatgctcctggacgagtcaatctttatttgactttggatgggaaaacaccaatcagtgaggttttgagttttcactatcgtatggtgcctgacagtcaaaacttggctgaggatgagccccaaaagtcaaagcttcagatgcagatgagactagctcgattgttgttcaccacaaacaagaaaaagatagcacccaagttacttgtagaaggaagcaaggtttccaatctcttatcagcatcgacagagaaggaatggatggatctgtcaaaatttgttactgattctaaaggcacttatgttccggccactgagggattgcttgaactagtactgcgtaatagattgcaagagtggcttgttgaaaaattaattgaaggccacaagtcgactggccgcgatgatctaggacaagggcctatccatctgtgttcttgcttgggctatacttgggccattcacttattttctttgtcaggattctccttggatttccgtgattcttctggttggacagctttgcattgggccgcatactgtggaagggaaaaaatggttgctgctcttttgtctgctggagcaaatccaagcttggttacagatcctactcatgatgtccctggtggacaaactgctggtgatctggctgctggacaaggttatcatggcttagctgcatatctttctgagaaagggctgactgctcattttgaggctatgtcactgtccaagggtaagcgatcaacatcaaggacagagtcactaaaacggaataccaaggaatttgaaaaccttagtgaacaagaattgtgcttacgagaatctttagcagcctaccgcaatgctgctgatgctgctagcaacatacaagccgcactcagggagcgaactcttaagcttcaaacaaaagcaattcagttggccaatcccgagaatgatgcatctgcaatagttgctgctatgaggattcagcacgcataccggaactacaacagaaagaaaatgatgagagctgctgcaagaatacagagtcatttccgaacatggcagattaggaggaacttcatgaacatgcgaagacaagctatcaaaatacaagctgcatacagaggtcaccaagtgagaaggcagtaccgcaaggtattatggtccgttggagtagtggaaaaagctatcttgcggtggaggaagaagagaaaaggcctgcgtggcattgcaactggaatgccagtagcaatggccacagacgcagaagcagcaagcactgcagaggaggactactaccaggttggtcggcaacaagccgaggacaggttcaatagatcagtggtacgtgtccaagctttgttccgttctcatcgggcgcagcaggagtaccggaggatgaaggttgctcatgaggaggccaaggtggagttcggtcgaaagtag

SEQ ID NO: 55 - DNA, 1041 bp, Zea mays
  source 1..1041: mol_type = genomic DNA
atgtcacggtggcgcgcggccgcctccagcatctccgccgccgccgcgtcccgcaactccgcggcggaggcccgcgtcctctccagggcattggcgaaggccccctcgccacctccgcttcctccccaaaccctctcgagaaccttcgccaagtccgctgccgccactgccaccgccacgtcgcccggccctggcgccacctccccgcccgccgggacgtggtccgagatcagactgaattctatgttcttatccaagccctgttcattagcgttgccacctgactctcctcatagagcagtggatccacaatttaaaggcatcaggcgattccttctcacactgttgttgttctacagtaagcaaagcaagtccatcagaggagcaaatgttgtttatgatcgaatcacttctcaagttgatacacctgccatttatgatgtttttcagttggagaaaacatttaagacaacattttcgttgcttgttctccatatgtggcttgttctacgccgcttgaaggaagaagggaaggatggtgttaagtttggacagtacatctacgaaatgtacaaccacgatgtagagctcagagtttctaaagctggggtgtttgatactttcttgaatattacatttgtgtcccagaggacaccaaaatttggatgttgtacagtggaagtagttctgcagttctttttggtttctgatttcaatgttaacttgctgttgacaaaatggatgaaggagctagagaaaatattctatggaaacattgtgaagtatgatgctgcgataagcccagaggctcatcaagatgatcttgtaaatgtcatctggagaaatatttatgctgaggaaggttcagaggctatggatgttgctgctgcccctgctgtgcaggcattggcaagatacacacgacgagaggcaacttgtctgtcattgactgacagagattctatgttctctggaaacttcaaattcacaaccctggtgccggcaactcccagccccagcccaaagaagcatgggcgatga

SEQ ID NO: 56 - DNA, 786 bp, Zea mays
  source 1..786: mol_type = genomic DNA
atggagctgacgtcttcttcccgctccgtgctctcctcctgcaaccggccggtcgctcgagctctttgcaggacggcgtacccgggtgcaaggaggaaggagaagggaatagtatgtatcttgagcgaggagaaagaagtggtacaaaggacgatccggaggcgagctgccttttctttgttgctggcttcgccggtgctatcagtggcgttctcggcctatggaaagagtaaaaccatgaacccttacgatgaaaggcgtctgcttcagcagaataagaagattcaggaagccaaccgtgcccctgaggatttcccaaacttcatcagagaaggcttccaggtcaaagtagtgacaccggacaactatattacacgggcctctggactaatgtatgaggacattaaagtcggtacaggcgactctccaaaggatggtcagcagattatattccattatgtgggctacaatgaagcaggacgaagaatagatagcacctatattcagggttcccctgcaaaaattcggttagggaacggaacactagttccaggaaaatcacatctcattctagatattgatacttcaggttttgaagaaggcatacgggacatgaaaccaggtggtaagagaagaattataatacctccagagcttggtcctcctgttgggccttcgacgttcttcagtgcgaagcagttcgaagtgttcgatgtggagctactggcggtgcaggactgccagcggaggacgatagcattttactctgatgtcgtgtgcagctga

SEQ ID NO: 57 - DNA, 2388 bp, Zea mays
  source 1..2388: mol_type = genomic DNA
atgccgagctcgcggcggaccacgggccgcgggggcgcaggcggaggcagcgcgggagcgggtgatgcaggaggcggcgacggcgaccgggtgccgccgttcatgggcaacaaccgcgaccacaacccgcgggagctgcgctcctgggcgcgccgcacggggttccacccctcggccttcttctcgggggagtccaactcctccttcgcctcctccgccgcgccgcaggccccgccgccgccgccgcctcccccggtctcctcgcgtcgcccgccgccggcacgggagcgggaacccgaaccggacacggaggacgaccttgttcccgcgccgccactggaattagagcgcggcgggcccgcccccgtccgcggacgcggcggacgcccgcgccgccgcatcgacctccgcggcgagctcgtgatcccaccgggcttcggccgcgaggaggcggaaccggacgcgggaaaggggcgcgcgcgcggggacgccaggaggaggaacttcggcgtcgagatggatcagacgccggccaatgcgggccggaacgggaacggagcgctcgcggacgcggagtcgaggaagaagtccgaggaggcggaggcggacgcgaagcggaaggcggaggaggccgaggcgaggaggaagaaggaggaggaggagagggacgccgagctggctgcgtactaccaggaacagtgggcgaacgaggatgagggagctgccgacgctgccgcggccgagacggccccgctgtatgaggcgtcgggactacggtgcggtgtaaccgataacccagggtgggcactactaatattttatggcatgcaacattacttgtcaatagctggttcacttgtctttggtcctttgatactcgtacctacaatgggtggttctgatgaggatactgcaacagtcatctccaccatgttattagtctctggtcttactacaatactacatacttttttgggttctcggcttccactgattcaaggaagctcctttgtatatttggctccagcgttggtaattgcgaactccgaggagttcagaaatcttagtgacaataaattcaagcacataatgagggaactacagggggctatacttgttggttcagtgttccagataatattaggatacactggtcttatgtcactgtttctgaggttaataaatccagtagtggtggcaccaactattgctgcagtgggtttggcattttttagttatggtttccctcaggctggcagctgtgtagaaatcagcatgcccctcattctgttggttcttctgtgcactttgtacatgagaaaaatatccttgtttggcaaccatatcttccttgtctatgcggtgcccctcagtgttgcaattgtatgggcttatgcattctttctaactgctggtggagcatataacttcaaaggctgcagctcaaacatacccagttcaaacatattgttggattcatgcagaaggcatttagagaccatgaggcgctgccgtaccgatgtttctactgcgtggaaaactgctgcctgggtgagggttccctatccgttccaatggggccctccaacgttccatttcaagacggttatcatcatgataatagtttctctggttgcttcagttgattcgctttcatcgtaccatgcggcgtcactgctggtaaatctaagcccaccaacacgtggagttgtcagcagagcgattgggcttgaaggggtttcaacgtttattgctggagtgtggggtacaggtacaggatcgacaacgttaacagagaacatacacaccctcgaaacaaccaaaatgggcagcaggagggctttgcagcttggggcagccgtgttagtcatcttctccttctttggtaaaatcggagctctcctagcctctatacctcttgccttggccgcctctgttctgtgcttcacctgggcgctaattgtcgcgcttggtttgtccacattgcgatacacccaagcagcaagctccaggaacatgataatagttggatttaccctgttcatctccttgtcgatacccgcatactttcagcagtatgaacccagctccaacctaatcttgcccagctatcttcttccatatgctgcagcgtcaagtgggccagttcacacagccagcagcgggctgaactatgcagtgaacgcgctcctatccatcaacgttgtggtggctctccttgtcgcgttgatccttgacaacacagtgccaggcagcaaacaagagcgcggtgtgtacatttggacagatcccaaatccctcgaggtggatcctgctacattggaaccctaccgattgccggagaaggtttcgtgctggttcaggtgggcaaagtgtgtcggcatctga

SEQ ID NO: 58 - DNA, 804 bp, Zea mays
  source 1..804: mol_type = genomic DNA
atggcttcggccaaggtggagaaccgaggcgcgctgtccaaaaggtcacgtaacgacgtgtctgtaagggagggggactggaattgtcctcagtgtggtaatgtcaacttcagttttagaaatgtttgcaaccgtggagcctgtggtgcaccccgtccatcaccgagtccaagtccaagaatgatgtcaggacctcctgcttctggatatgaccggtcacctctattttacggaggaggtggtggcgccccacctccaattccgcttggaactggtagctatggtgctccatatccacaccttgggatgcgatatggctatggtccaccagtaggacctcctggttcatacggtcttttctcttcatatggtccacctgggccaatgagtggtatgggatatggcccaggacctgagctaggccgatatagctatgggtttagaggttctccaatgccggtttctagtccatggtctggtggagcattagctgaaaacaatgacaacattgcttcacgaaagcgccgtggaggccctgatggactttctgagggtgactggacctgtcccaaatgcgacaacattaacttttccttcagaagcacttgcaacatgaagaaatgtggagccccgaggccaactccgggaggtaacacgagctcatctcgcaaggacaacagcaataaggaagcccctgaaggaagctggacctgcccagagtgcaacaacctgaactacccattccgcacggtgtgtaacaggaaaggatgctcgtacagcaagccagccccggcgaacaactga

SEQ ID NO: 59 - DNA, 285 bp, Zea mays
  source 1..285: mol_type = genomic DNA
atgttcgcctggatcctccgcggctgccgcgacgagtgctccgcctccgaccagctcaagcaggctcacgatgtcttcaaggcgaaagaggtggtcctgcaaaagaagatctctcaagaggtggaacgggccaaggagttcacaaagtcgggaaacaagcaggcagcaatgcagtgcctgaagaggaagaggtactacgagagtcaaatgaaccaagttggaagcgttcagctgcgcattaacaccaaggagagaatgatcgctgatcacacgggcaacaaataa

SEQ ID NO: 60 - DNA, 738 bp, Zea mays
  source 1..738: mol_type = genomic DNA
atggccatggacgcggagcggcggcaagcggagctgatcgcgcagttctcctctcaggcggtggctctttcgtccgcgcagcagctcgccgctctggtgctcgaggccacctcccacccggccctcttcgccttctccgagctcctcgccctcccggccctctccatgctcgctggcactcagtactcgtcttcactggacttgctccggctcttcgcctatggcaccttgaaggactataagaataattcgggatcccttccagcattgttgcctgatcaggtccgaaagctaaagcaactcagtgtgctaactctggcagagtcaactaagatactaccatatgatcagctcatgcaagagttggatgtttccaacgtaagagaactcgaagattttctaatcaatgagtgcatgtattcaggtatcgtcagaggcaaactggatcagctgagaagatgctttgaagtacaatttgcagctggaagagatcttacacctgatcagctaaacaacatgatagagatcttatctgactggttgggaacatcagatagcctgttgcatcaaattcaggagaaaatcaaatgggctgacacaatgagtgatgtgaacaagaagcaccagaaagaatttgaagacagagtggaagaggccaaaaaatcaattaagttgaacaatttaagcaggcagacatcgacatacgagggcatgacgactacctctctgaatctgtag

SEQ ID NO: 61 - DNA, 1068 bp, Zea mays
  source 1..1068: mol_type = genomic DNA
atgccttttctcacagtacctgaatcttacactatgatctcggagtcgaagtttcaggtgcctgtggaggttctctttaatttcttgttgtctgatggtgcttttggctttgtggatgatttccacacaaaatgtggtgacaaagaatttagttgctccaaatggcgtacagatgagcaagggggacttgtaagggatgtctcatttttgcatccaataaaaatttatcttggtgcaaaatttgggacttgtcaagaggttcagaaacttcgtctttacaaaaacaggcgtctagtcattcaaacctctcaatcaataggtgatgccccatatggggaccattttactgtagagggcatctgggatgttgaacaagatagcctagatgagaactgttgtgacctcaggatatacatcaatgtagccttctcaaagaaaacaatatttagagggaaaatagagcagtcaacaaaagatgaatgccgggaagttttcagtctctggattaagctcggtcatgatcttttgaagcaggagtatgatcgatcaaaaggcgcctccagcacacctgattctggtgttcaaccaggggctacaacaaacgaggaaagtgccgtggaagtagcagttccagtggtgagttcctcacatgatgaatctggtgcgagaagcctcattcctcctactcaagatcaccagcagaggacaggaagagatccttccatcacgtccacttcacaagaattgtggggttctctatcgtcatacatgaggtcaaggcaacttggtccagtactagcaatgtcactggtagctctcattatcctaatgcaggtgaccatcatagttctactaacaagacccccccaggtccaaatggatcctcatgggatctggacgggcagttcagggtacagcaaagaaagcatagagtgggtgcagaagcgcctcagcttactaagcgaggagatgcagctcgcggaggcacacatggagacgatgaggcgcgagtttgcctggctcaggtcttacctggagagactgcagaggacgcggggcagcacatga

SEQ ID NO: 62 - DNA, 1317 bp, Zea mays
  source 1..1317: mol_type = genomic DNA
atggcaatggtcaccgccacgatctcttgctcttatccacagctccggtatcgcccctctcctcgatgccggcgccacggccccgcagcggccaccgtggccgccttggcgacgccgcggtcggagcaggacatcgtcatcgtcggcgcgggcgtcgcggggctggccacggcggtggcgctgcggcggctcggcgtgggcgccgccgtgctggagcagggcgacgccctgcgcgcgggcggcacgtccctgacgctgttcaagaacgggtggcgcgtgctggacgccatcggcgtcgccgacgagctccgccccaagtacctccgcatccaggggatgaggatgaggtcgccggggcgggatctgcgcgagttctccttcgacgaggaagctcccgggcaggaggtacgcgcggtggagaggagagcgcttctcgagacgcttgcttctaagctgccaccaggcgccatatcgttctcgtcgaagctgaaatcgatctcggagcaagggcgtgcaggcaccctgctcgaactggaagacgggaggcagattcttgccaagatcgtggtcggctgcgacggagtgaactcgccgatcgcgaggtggatgggtttctcggagccccggtacgtcgggcacatggcgttccggggcctcgcggagtacgccgacggccagccgttcgagcccaaggtgaactacatctacggccgaggcgtccgtgccggctttgtccccgtctccgcaaccaaagtctactggttcatctgcttcaacagacaagatccagggcctaagatcaccgaccccacggcgctcaagaccgaggcgctggagctcgtccgcggctggccatcggacctcctggcggtgatgcgcagcacgcccgagggcgccgtggtgaggacgccgctggtggaccggtggctctggcccgggctggccccggccgcgtcgaggggcggccgggtggtgctggccggcgacgcgtggcatcccatgacgcccaacctcgggcagggcgcctgctgcgcgctcgaggacgccatcgtactggcgcgccgcctcgccgacgccggcgcggacggggcccaggccgccgccgccatgcgcgcgtacgaggcggagcggtgggcgcgcgtgttcccgctcacggcgcgcgcggggctggtgggtgcgctcgtgcagtgggagaacgcggcggtgtgcgcggcccgcgacggcgtggtcatccccaggctcgtcaggctgggaccgttcctggagcacaccaacttcgagtgcgacctgctcgaaccggcgccggagtcgccatga

SEQ ID NO: 63 - DNA, 462 bp, Zea mays
  source 1..462: mol_type = genomic DNA
atggagcagaccttcatcatgatcaagcccgacggcgtccagcggggcctgtttcttgcgcagatcggggacatcatcagtcgcttcgagaagaaagggttctacctcaaggggatgaagttcatgaacgtggagaggtccttcgcgcagcagcactacgctgacctttccgacaagcctttcttccccgggttggtggagtacatcatttccggccccgtggtggcgatggtgtgggaggggaaggacgtcgtgttgactggccgcaggatcattggggccaccaggccttgggaggcagcccccggtaccattcgtggggactacgccgtggaagtcggcaggaatgtcatccatggaagcgactccgtggagaacgggaagaaggagatcgctctctggttccctgaaggtgtggcacagtggaagagcaaccttcatccctggatctacgaggcttga

SEQ ID NO: 64 - DNA, 987 bp, Zea mays
  source 1..987: mol_type = genomic DNA
atgggcaaccttcacgccaaaggtgctggaacgggcgccaaaggtgctggagcagcaaacacttccatctcgaattctcgggactcgatgtccaatgccgccacaccttctccagtagagcaggcaaccttcaagtgggtcattgatggcttttcctccctgcttgacaaggatcaaggatggacatactccaacgtatttgagctcatgggtgtagaatggtatctgaaactgaatccaaagtacgaagtcagtaatgaggagtgtgtctctctcaggcttgaactgtcgcagagttcagtgaaactcgacactatctttgaggcatctttcaagttcatgatatatgatcagttaatcggaaagcacaaggtgcatctaggtaaccacagcttccagactgcaagtacgagctctgccatcgcattcatgttacctctgaaagcactcagacaatcctctagatttattgtcaataacagctgcgtatttggcattggcttcatcaaagttgccactatcaaagttaacacgacgctagaaacactgtttgtcaggaagatgaacatcttcaacgaggccaaagtttacacttggaaaatcgaggatttctctgctctgaagaacccgtcccactcaccggagtttgagatcgctggatacacatggattatctctttgaatccatcctacgatggtaacagcctgtccttattcctgaaaatgaagaagacaaacgacgtccccaaaggctctgggagcctggtcgagttcgctttatccatcaaagaccaggaaaacggcaaggacaggaaatatccaggcaggtgccagttctcgagcaaacaccatcgctggggatggaagaagttcatctcgctggaggacttcaaggactcgtcaaagggttatctcatcaagggcaagtgctgcatcgaggctgaggtcgcaatcagtggctcttcgaagacggagtactctatgtaa

SEQ ID NO: 65 - DNA, 249 bp, Zea mays
  source 1..249: mol_type = genomic DNA
atgggtgttgctgagatcaacattactgacatctacgatgctgcgaagctggacctgaagcacgcctctgatgggacaaggatcaagacgatctaccctgtcggagtaaactaccttggcggcgagagccatgttcagtggaaggatggcaaggtggtccaggacttgattctgaagctgaggaaggtcgagagtggcctgattgtggtgcagctggagtgggttcatgttcctggcgtgaagctgtga

SEQ ID NO: 66 - DNA, 2322 bp, Zea mays
  source 1..2322: mol_type = genomic DNA
atggcctcttcggtggccgccatgccacacgagtccgcctcatggagggacccctcccgccccaccccgtccagaggcttcttcaacatcctcatcccctcccccgaagccgcagcggcctcctcctcctccgccagcgagcccacgcccaggcgccgccgccaaatcctcgaccgctgggcgaccgccgccgccgccgtcacggccagcgccccgccggcccccgccgaccagcgccgccgcgccagggacgccgagctgtccgagctcgcttccgccacgcgccccgtcgccgcgcgcgccgccgtcttccgggagccgtccccggcgccctccgacgcctcctccacggccgcgcccgcggctccggcggcggcggcggcggccaccgccgccgccgttgccacgccctccgagctcccgcccgcggggccccgggcctcgtcgctcatccagcgctggcgcgcgatcgaggccgtcgggcccgccacgccgcgccccggtagcgccgccgaccccgccacgccgcgccccggcagcgccgccgacccggcctccgcctccgactccgacaccggctcgccgcgcggccgcgtcggctgcatcgtcaagaagctcagcggggcgtcgtcgctgcccgaggatgagctcgacgacgccgccaagtccgagctctcgctctcgcagtccgcgcccccgtcgcccgcgcccacgcgcgacgcctcgcagtacccgccgtacgcgtcgggcgcgatcaactgcccccgcccgccgcagctcgtcgtccggacggtgcgcggccgccgcgccatggaggagctcgtcgccgccatcgcgcaccggcggaggcgcgaggtcgccgcgctcgccgagaggcacgccgtgtcccgcttcgcgcacaagggcaggatccagtccatgcttcgtctgagattgttacgccagagaggtacagttgaggatgaaccgtggagtctactaaagccggttaggccacaccaacaaaaacatgtgggcaagttgaggtacgatagcagggacacagacttacaaaaagcaaacaattcctgtcaacaaaataatagcaagtgcatgcctgacgaacaactttataacgatagagttccagctgaggaaaaatcaaatgatatatccgctgaaggccttgttgatggttcaggaaatctacagtgtgatgagcagatgaaagcaaagggggatacatgtcagaaagattgtgaaaacttttgtgttcatagtcaacactattctgaagcatcaagctttgcaaggtatggtgagcatagtactatcgatgagaatcagtttgtggaagatatctccccaagcactacaagcacgttgcatgaactagaaactccatcgtccagaggagataatcttcgcgaagaagacaaccaaagcataaacggttcctgggaagagagagggttatggataagcagcctaggttggcctgcaccaattgatactatgagtccggatagctggcatcaggaaacaatgggtgatattgagaatcataataatccaattcaattcaatgaccgcccctggatagagtcccctaactcgtggagatcactatgtgttgtcacgcaatcagactgtcatgctctgtcacacaatgctgacatttgcaaccttcttgagagaaaaaaggtctcaaagtctcttgagagtgacttcagcaataaaatgaaccagctgttgctaacagctctgcaaaaacaaaggcaacaacggatgatggatgactttggaggatactatgatgaacgcatgtactggagacaaaatgatgaaattcgtgatgctgataaggaggcatctgctccatgttcattagcccctgtcgcacatcttggagctcatcagcaagagagttggcagcattcttcatttggaagtcaacatcaacaccacgataaccaaaatttacttgaaatggaagtcagggtaaggggtgaaatgtcccagattcatcaggaaatatatcaactgcggaagttggtggaaagttgcattgcatcccaagtaaagatgcagcactccattaaagaagaggtgtgcagtgcacttcgtgaggcaggcttgatgcgaagccaacccgacacagcagcagcaaaaaggggtaactgctgcatctgccatcgaatgcaagttgactctctactttacaggtgtgggcatgtgtgcacctgttttgactgcgccgatcagctgaaatcgagcggcatgagctgcccaatctgtcaatctcctattgatgatgttgttctggcgcggttgaatttttga

SEQ ID NO: 67 - DNA, 1095 bp, Zea mays
  source 1..1095: mol_type = genomic DNA
atgaggacgccgcggggtgtctcgtcgtcgttccttgccctctacgggttccttctcctcgtcgccgtctcggtggccgcgaccggctacgccgcttcgccgggggagagttccaacggttacggcgctgcagccggttcgaacggcggcggccaggagggaagctacacggtgccgccgtaccagaagtccgtggacggcctcgacgagcggtactacgagaggtcgtgcccgaaaatggaggagatcgtcgggacggccgtgatgaaggccgtgaaagccgacgagaccctcgccgccagcatcatccgcctcttcttccacgacttcgccgtcgggggcgtggacgggtcggtcctggtagacgtgcccgggcagagcgagaagtacgcggaggcgagcaggacgctgcgcgggttcgagctgatcgaggggatcaagaaggaggtggaggccaagtgccgcgccaccgtctcctgcgccgacatcctcaccgccgccgctcgcgacgccgccttctcggccggggcgccctactggtcgctcaagtacgggcgcagggacaggaaggactactccagcgccgaggcggccgaccgcgacgtccccatgggcggccagcgcgtcaccgagctcgtcgccttcttcgatacgaagggtctcaacatccaagacctcgtcgcgctttccggagctcacacgatcgggcgcgcgacgtgcggatccgtgaggccggggctgtgcaagcgccggaaggcacgcacgctggaccggcagtacggcgacttcctgcagcgcaagtgccgcgccggcggcgacgcggcggaggcgtacgtggagctggacggcgagacgcccacggcgttcgacaaccagtactacaagaacctgctgcacgggaaggggctgctggacacggaccaggcgctgctggcggactcccggacggccgggttcgtcaagtcgttcgcgtaccagaagccccaggagttcgcccaccagttcgccaagtccatgcgccgcctcggcgaggcgcaggtgctcaccgggaacgagggcgaggtccgccgcaagtgctcggccgtcaactactaa

SEQ ID NO: 68 - DNA, 1227 bp, Zea mays
  source 1..1227: mol_type = genomic DNA
atggcaccgccggcgaggagcccgacagcggggcctcggcggcgcgccctcgtcgtcctcgccctcgccctcctcctgcttctgcccctgctgctgctcctccacctcatctcctcgccttcgcctcgacacctccccgcgccccgcacgccctcccagtctcaggcctgcgactactccgccggcgagtgggtgcgcgaccccttcgcgggctcctcgctccgctacgaccatacctgcaaggagatcttcaagggatggaactgcatcgccaatggcaagggtaacgcccgcgacttgctcagctggcggtggacgcccgcggggcccgggtgcgagctcccccgcctcgacccgcgccggttcctggagcgccacagggacaccagtatcggatttgtgggtgattcgttgaaccgcaacatgtttgcctcattagtctgcatgctcaggggagtcaatggtgaggttcgcaaatggcgtcctgctggggcagaccgtggcttcaccttcctacgctataacctcactgtggcataccacagaacaaatctcttggtacgctacggtaggtggtcaaggaatccaaatggaggccctctagaatctcttggatataagcaaggttacagagttgatgtggatattcctgaccaaacatgggtagaggcatgtagcttccatgatgttcttattttcaacactgggcattggtggtgggcaccctcaaaatttgatccaatacggtcaccaatgctgttctttgagaaggggaggccaattgtacctcctttactgccaccggctggactggatctggctcttcaaaacatgatatccttcgcgaacaaagcaacaagaccaaacacagtgaaattcttccgcactcaatctcctagacatttcgaaggtggtgactggaatgaaggtggatcttgccaatgtaatcagcccttgtcgtcaaaagaggtggaagaaattttctctttggacaataacagcactaatgtagaagcacgcttggtaaaccaacacttggtgaaggctcttcaacagactagtttcagagttctggacatcactcgcatgagtgaattcagagctgatgcccacccttcaacaactggcgggaaaaaacacgatgattgcatgcattggtgtttgccaggaccaacggatacatggaacgatttgctagctttgagtctcgaggcacttgagagctag

SEQ ID NO: 69 - DNA, 3783 bp, Zea mays
  source 1..3783: mol_type = genomic DNA
atggcaatgacgcggccgttcttggcgccgctgatgatagtagcggctgttcttctttcacgcatggcggcggcggcagcggacgacggcgacgtgctgctgcaggtgaagagcgcgttcgtcgacgatccccagggggtcttagcgggctggaacgacagtgctggctccggcgccggctcgtcggggttctgttcctggtccggcgtggcgtgcgacgcgtcggggctcagggtcgtgggcctcaacctgtccggcgccgggctggccgggacggtgtcccgcgcgctggcgcggctcgacgcgctggaagcgatcgacctgtcgtcgaacgcgctcacgggccccgtcccggcggcgctcggtgggctacccaacctccagttactgctgctctactccaaccagctcacgggccagattccggcgtcgctgggggcgctctcggcgctccaggtgctccgcttgggagacaacccgggcctgtcgggcgccatcccggacgcgctaggcaagctcggcaacctcaccgtgctcggcctagcctcctgcaacctcaccggcccgatccctgcgagcctcgtgcggctcgacgcgctcacggcgctgaacctgcagcagaacgcgctgtccggcccgataccgcgcggcctcgccggcttggcgagcctccaggcgctcgcgctcgccggcaaccagctcacgggcgcgataccgccggagctcgggacgctcgcggggctccagaagctcaaccttgggaacaactcgctggtgggcgccataccgccggagctcggcgcgctcggcgagctccagtacctcaacctcatgaataatcgcctcaccggccgcgtcccgcgcacgctcgccgcgctctcgcgcgtgcacacgatcgacctgtccggcaacatgctctctggtgcgctgcccgccgagctcggccgcctgccgcagcttactttcttggtgctatccgacaaccaactcaccggcagcgtccccggcgacctgtgcggcggcgatgaagcggagtccagtagcatcgagcaccttatgctctcgatgaacaacttcaccggggagataccggaggggctctcgcggtgccgagcgctgacacagctcggcctggcgaacaacagcctctccggcgttatccccgccgcgctcggcgagctcggcaacctgacggatctggtccttaacaacaacagcctctccggcgagctgccgccagagctcttcaacctcactgagctccagacactggcattgtatcacaacaagctctccggtcgcctgccggacgccatcggccgtcttgtgaacctggaggagctgtacctgtacgagaaccagttcaccggcgagataccggagtccatcggggactgcgcgagcttgcagatgatcgatttcttcgggaaccggttcaacgggagcataccggcgtccatggggaacctgtcgcaactgatcttcctcgacttcaggcagaacgagctgtccggcgtgatcgcgccggagctcggtgagtgccagcaactcaaaatccttgatttggccgacaatgctctgtccgggtcgattcccgagacgtttgggaagctccgttcgctggagcagttcatgctgtacaacaactcgctctccggcgccatcccggacggcatgttcgagtgccggaacatcacgagggtcaacatcgcgcacaaccggctcagcggcagcctcctgccgctctgcggcacggcgaggctgttgtcgttcgacgccaccaacaactccttcgacggcgcgatcccagcgcagttcggccggtcgtcggggctccagcgcgtgcgcctggggagcaacatgctctccggaccgatcccgccgtcgctcggcggcatcaccgcgctgacgctgctggacgtgtcaagcaacgcgctcactggcggcttcccagcgacgctcgcgcagtgcacgaatctcagcctcgtcgtcctcagccacaaccgcctgtcaggggcgattccggactggctgggctcgctgccgcagctcggcgagctgacactctccaacaacgagttcaccggagcaatcccggtgcagctcagcaactgctccaatctcctgaagctgtcgctcgacaacaaccagatcaatggaacagtgccgcctgaactcggcagcttggcgtccctcaacgtgctcaatctcgcacacaaccagctctcaggtcagattccaacgacggttgcaaaactgagcagtctctatgagctgaatctgtcacagaattatctgtccggtccaatccctccggatatcagcaagctgcaagagctgcagagcctgttggacttgagcagcaacaatttcagtggccacatccctgcatcactcggctcactctccaagctggaagacctgaacctgtcccacaacgctctggtcggcgcggtgccgtcgcagctcgctggaatgagtagcttggtgcagctggacctgtccagcaaccagcttgaagggaggctgggcattgagttcggccggtggccccaggccgcgttcgccaacaatgcagggctctgcggtagccctctgagaggttgcagcagcaggaacagccggtcggcgttccacgcggcgtccgtcgcgttggtgactgcggtggtcacgctgttgatcgtgctcgtgatcatcgtgcttgcgctgatggcggtgcgccgccaggccccggggtcagaggagatgaactgctcggcgttctcgtcgtcgagctcgggcagcgcaaaccggcagctcgtcatcaagggctcggcgcggcgggagttccggtgggaggcgatcatggaggccacggcgaacctgagcgaccagttcgccatcggatccggcgggtcgggcacggtgtacagggcggagctgtccactggcgagacggtggccgtgaagaggatcgcggacatggacagcggcatgctgctgcacgacaagagcttcacgcgggaggtcaagaccctgggccgcgtccggcaccggcacctggtcaagctgctcggcttcgtcacgtcccgcgagtgcggcggcggcggcggcatgctcgtgtacgagtacatggagaacggcagcctctacgactggctgcacggcggcagcgatggccggaagaagcagacgctcagctgggacgcgcggctcaaggtcgccgccgggctcgcgcagggcgtggagtacctccaccacgactgtgtgccgcggatcgtgcaccgggacatcaagtccagcaacgtgctcctcgacggcgacatggaggcgcacctcggcgacttcggcctcgccaaggccgttcgggagaaccggcaggccgccttcggcaaagactgcaccgagtcaggttcctgcttcgccggatcatacgggtacatcgctccagagtgtgcttactccctgaaggcgacggagaggagcgacgtgtacagcatgggcatcgtgctgatggagctcgtcaccgggctcttaccgaccgacaagacctttggcggagacatggacatggtgaggtgggtgcagtcaaggatggacgcgccattgccggcccgggagcaggtgttcgatcctgctctgaagccgctggcgccgcgtgaggagtcgtcaatgacggaggtgctggaggtggcgctccggtgcacgagggcggcaccgggggagaggccgaccgcgcggcaggtctccgatctgctgctccacgtttcactcgattactaccgcgcctgcgaaaaacgttag

SEQ ID NO: 70 - DNA, 243 bp, Zea mays
  source 1..243: mol_type = genomic DNA
atgccggcgacgccgacgatcatcggtgccctcctggggctgggcacccagatgtactcaaacgctctccgcaagctcccctacatgcgccatccatgggagcatttgttggggatgggtctcggtgcagtgtttgtgaaccagctggtaaagtttgatgagaagctcaaggaggacctcgacaagatgcttgagcgcgccaggctagccaacgagcagcgctacattgacgatgatgaatag

SEQ ID NO: 71 - DNA, 912 bp, Zea mays
  source 1..912: mol_type = genomic DNA
atggggcaccactgctgcagcaagcagaaggtgaagcgtgggctgtggtcgccggaggaggacgagaagcttgtcaagtacatcaccgcccacggccacagctgctggagcgccgttcccaagcatgccgggctgcagcgctgcggcaagagctgccgcctccggtggatcaactacctccggccggacctgaagcgcggaaccttctccgaccacgaggagcgcaccatcatcgacgtccaccgcatcctcgggaaccgctccccgaccaaggccgccgcctacggcaccgtcacggccacgccgccgccggagacgatggcgccggcaacgtacgacgtccctaataattaccctgacggcatgctcatgaggcatgatcaggcagccacggcggctgcgattccgggatacccgtaccaccccggaaacggcggcggcgtgctgatgagcttcagggaccagaatgctggtctccagacatctgtggacttcgtgaacggctcctcgtcctcctcctccatggatcacgccgccggcatgcctaacggcaacggctttagccaacaaggcatgggcgcggccttcatggacgtgacggcgggaatgtggagcaccgcagccgtggattctggcatgtgtgcagggatggaagtggctcagccaccgccgccgccgccgcagcagccgctgccaccacaagggctggtgcagggggaggtgctcgtcggccgacaaactgtgatgaacggtggcggtgcagttgctgacaagggcatggacatgatggacgtctcttcagtgtatggcggcgccggtgcaatggcgttcgatctggagctgatggagtcatgcgcgttgttttgcggcagcaccggcaacaccatggagcagctgcaatgggactgctaa

SEQ ID NO: 72 - DNA, 312 bp, Zea mays
  source 1..312: mol_type = genomic DNA
atggcggttgtggaggcgtgcgaccccgcaaggatcgacatctcgtccaagacagcagcctcctcagagcggacgacgcagcttatctctggtggaaggcacgcagccacgcccccactcccctcctcgtgctccccgccgacgcctgcctcgcgctcctcgtcgaccgtctctccctctcctccgtcgttgcatctcgcctctcgcgcacgcacaaagctgaggacgttcctgctcaagccgttcaagctcatcaccgtcttcctccacgagaccagccacgcgctcgcctgcaagctcacctgcggtgat

SEQ ID NO: 73 - DNA, 603 bp, Zea mays
  source 1..603: mol_type = genomic DNA
atggcggtcccgaggcctcgaatccgcgccctacgtcgaggcacgctgctcctcttcctcctcgtcccgctcatctactccggacttagagctctgaacaagattggcctatcaagtgaagagaactattctggagaacatgtttcttttgttactgtatttacaacctacaattctgtctcagctggagatggcaatgtttcacctgattctgtaactgttggaaatagttcttatagcaaaatagaaagatccatgaccattctgaacactttcatcagtttcataaaggtatcaatgccaagaagcgatgtgatcatattgactgatcctggttcaaaattttcagtaaatcaagggagtgctacactattgcctattgaaggaaactattctcgaggaaatttgatgcttcagagaataaagacatacattggatttctggagcaaaagcttgtggaatttgataggacggagaggttgaatcattttgttttaactgattccgatatagcagtggttgatgatcttggacatatatttgaaaaaatccacattttcatctggctgttacttttcgtaataacaaagggcaaccattga

SEQ ID NO: 74 - DNA, 6615 bp, Zea mays
  source 1..6615: mol_type = genomic DNA
atgaaggagaggagctctttatgcgaaagtgcagccgatggaaactggggtttgaaatataaaaggaaaagaagcaagttaacagttagcccatctaatgagaatgaggctacctcaccaacatctgactctcgaatgagccatggttccaccaaaaagaagttgaagcatgacaataacatttctccatcagccaagaagataagagggcacgatgggtattttttcgagtgcgtagaatgtgatcttggtggcaatttgctatgttgtgatagctgtcctcgtgtttaccacttggaatgcctcaatcctcctcttaagcgtgcaccccctgggaagtggcaatgtccaagatgccgtccgaaaaaagttagcttgaagctgttaggtaatgcagatgttgacacttctaaacgtgaaaaaagtacaaggacatatgcaagtacggcatcagatagtccaccttcgcatacaaaagtctcctttaagaccaggaactccacacaagacaaaactggattaaacgaacaaggaaaagagtcatctggtggcacaatgaatgggggtgaaaccagtacgaagaataatgaagttgagaaaaagaagacactgatattacatttgaagaagcgttcaaccaaggaattgtcagagaacactaaaccctcgaagtcagaatttgttgaactttcagaagagaaaattgtaaaacatggaagtgttttgaaactcaagaaacatccacatcgtatggacttgtctccaagtaaatctaaaaccaggaggcaaagtagccagagagagagtaaaagatctggcacaaaaaagattaaatattccatgtctgatgatgacagtgtttcctcatcagaaccatcgacatctcttgataacagtgaatcaccaccaaaaagaaaacactcagatggaaaagcaccttcatccagtaccaagaaagggaaaaagaaagtgaaatttgttgagaaaaagcactctgaggagcagggcgtggtaggtgacaaaatgatgactacccaggaggatcagcaggtcgatcgcatcctaggctgccggcttcagatgagccaaatcaacccgccgtcccatgcttcattggagcagtttgaatcagctaatctccaggtggatggtatggttcttccacatgcttccgctggcattcctaataggcctcatgatggaacagagtcttctagaaatggtacaattgaggatatatgtgctgatgaatcagccaatcatagtggtcacaatttagaaatgcgaaaggatacggacagtaaatcccatgaaaaggagtcacataagcgagaacaagtcaagaagatattatcagtatgttctggtgatgagaccaatataatgaaagatgatcaagtagtcagagaaaatgtttcagcttccaaaaaaggtgaagatgagacatctgatttgccagcagaaaaggatgataccatacttccagtcaccagagcttgtacaatgatcaggaccaatcaagatcatacagatgaaagtaaacagcatgggaaaatagaacaaataacaaataaggactacaatgatgctggatatgagttcttgatcaaatgggttggaaaatcaaatattcacaacagttgggtttctgaatccgaggtaaaaatattagcaaaaaggaaactagaaaactacaaagcgaagtatggaatgagtttgataaacatttgcaaggaacagtggtgccagccacagcgagttattggtcttcgtgcttctgtagatgaagtggaagaggttttaatcaaatggtgtggtcttccttatgatgaatgcacttgggaaagatcagatgaacctaccctgatgaaatatagccatttggtcactcagttcaagaattttgaatgccaggctttggataaagatgtggtaaaagattatgcaaatgcaaggaaccgacatgagcttaatgtgttagttgatcagccaaaagaacttcagggcggcatgctcttccctcatcaattggaagcgttgaactggctgcggaaatgctggtacaagtcaaaaaatgttatccttgctgatgagatgggtcttggaaagacagtgtcagcttgtgcttttctatcatccctatattgtgaatttaagattaatttgccatgtcttgtcttggttcctctttctacaatgcccaattggatggctgaattttcatcttgggctcctcatttaaatgttgtggagtatcatggttctgcaaggtcaagatctatcattcgccagtatgagtggcatgcgggcgatgcaagccagattggcaaaacaaagagatcgtacaaattcaatgttttgcttaccacttatgaaatggtgcttgttgatgctgcatatcttcgttctgtctcatgggaagttcttatagttgatgagggacatcgcctgaagaattctagcagcaaactctttagtttacttaattcattctcatttcagcacagagttttgttgactggaactcccttacagaacaacattggtgaaatgtataacttattgaacttcttacagcctacttcttttccttctctatcatcatttgaggagaagtttaatgaccttacaacagcagaaaaagtagaggagctgaagaaacttgtagctcctcatatgcttcggagattgaaaaaagatgcaatgcaaaatatccccccaaagatagagcgaatggtacctgttgaattgacatcaatccaggctgaatactaccgtgctatgcttacaaagaactatcaagtactgcgcaacattggaaaaggtggtgcacaccagtctttattgaacatagtaatgcagcttcggaaagtctgcaaccatccctatcttattcctggaactgaacctgaatctggttcacctgagtttctgcatgaaatgcgcataaaagcctcagcaaagctaactttgttgcatttgatgctaaaaatattacacaaggaaggccatcgtgttcttattttctctcagatgacaaaacttcttgacatccttgaagattatctgactttagaatttggccctaaaacatttgaaagagttgatggctcagtatctgtggcggagcgccaggcagcgattgttcgctttaatcaggataagacacgttttgttttcttgctgtctacacggtcatgtggacttggtattaatttggcaactgcagatactgttatcatatatgattcggatttcaacccacatgctgatatacaggcgatgaacagggcacacagaattggacaatcgaacagacttctggtttacaggcttgtagtacgtgctagtgttgaagagcgtatcttgcaattagccaagaagaaattgatgcttgatcaacttttcgttaacaaatcagaatcacagaaagaagtggaagatatcattcgctggggtacagaggaactctttggaaatagtgacagtgtggatggtaaagataacagtgaagtcccaggtcctgttgctgatgttgaatttaagcatagaagaaaaactggtggcctaggtgatgtttatgaggacaaatgtattggtggttctaccaaacttgtttgggatgaaaatgctattttgaagcttcttgatagatcaaaccttccatcatcaatggctgaaagcactgacggggatttggacaatgatatgcttggtactgtgaagtcaatagattggaacgatgagctcaatgacgaccctggtaccaatgaggatactgcacctattgacaatgatggtagcgagcaggcatctgaatcaaagcaaggtgctaccaatcgttcagaagaaaatgaatgggataaacttttgcgtgtcagatgggagcaatatcaaatagaggaggaggcatgtcttggtcgaggcaaacgtttaaggaaggctgtttcttacagagagacattcgcaacacttcctaacgaagctttaagtgaggactctgatgagggagatgaaccaaagagagaatatacagcagctggactggccttgaaggaaaaatatggaaagcttcgagctagacaaaaggaaaggattgctcagcgacacattatcaagaactatgctgatgataatcttgaagagttcatgactccatacgactctattgctaatgaacctgctgaaaatcctttggtaatcgtagaagatctaaattcttcacagctatctggcgcgaaacgtttcagtgactcaactggagaaatgaggcaatcctccaagaaatcaaagagatattctgaaatccctcaagatctttatgccaaaattcctggtaacacagcttcattcaagcaccactcaaaggccactgatgtattcaatccagggaccccaaatcacttgctacctgtacttggcctttgtgctcctaatgctgatcaggtgaactcatacaaaaactcaatttgtgcaccaagcataaaggagcgtaaaagggccagtggtgacattgtcaaacaactgtcaactcctgctgaccattctaatgtgcatagaaacgaagctcaatcagcttctgataaggccatctttcctggtgcatctgaggaggctttgcgaagaataagtaacatgattccagatagttattttcccttcagtcatattcctcctatatcagggaagggggttgatcctgttgaaaatcctggaccctccattgcatcatttcaagggaatctgggtcttccaaacttcagtctggaagacaacgctccacttaagcatattaagcctatacctgatatatttcctagcctgtcactgggtgctcataaggattatattcgtagttctgtcccagagttgccagatggttcattgctgccaaacttcatggcagatattactggtacttcgaagcaaaagtcatttatgtctggtctacttcctgggctgggtcttagcccagggcaaccaatacattcggctatgcctgataatcacaagaaagtcttggacaatataatgatgcgtgctcagtatgcgtcaaacaaattcttgaagaagagatcaaaactagattattggtcagaggatgaacttgatgctctgtggataggagttcgtaggcatggaaggggaaattgggatgctatgcttagagatccaaagctgaagtttttgaacaaccgaactagtgaggagctggccttgaggtggattttggaggagcaaaaaattatagatgaacctatgtctacatccaccaggcgttctaactcagcagcatttcctggaatatctgatgcaatgatgtctcgggcactgaatgaaagtaatttctctaaaatgaggatggagcagcctaaattgcagtcccacttaactgatattcagttaggttcgagtgatatcctatccacatttcctcatgttgacgctgctagttacattaactcaggggagggtggcccatctcaaataccatggcaggattttaagcatagatcaagttatgctggagattttcctgccggtgcatttgacaagttggagaaacctgatgttggaccgatcccgccatttatgccaaatcctttcatgactgacagcattggttcacttcccataaacagaaaaaacaatagttccatcccgcatagtgaaatcaggtcaagttctcgtgagaacattctttctggtgtttcggatggacagatcaacttgcttcatgagatgcagcgtcgtcgtcgtgtgaggtcaggcaaagaacccattgagatgaatctcaaccacatagaccactcaaatctccagcttgataatactagtgaccttggaggtttaaaaccaaacaagttacctcattggcttcaggaggctgtacgtgctccttcatctaagccacctgaacgtgagctaccagcaacagtttcggcaattgctcagtcggtctgccttcttcttggagaacaagaaccatcaatacctccattcttgatcccgggaacacctctttctcgcccaaaagatccaaggattaattcaaagaagaaactccgcaaggctcagcaatctaactctcatgtagagcattcaaaaattggatctggtgagggtgattgtgtcactaccccagcacccccatccattgaagcttcacctgcacttcctgtcgattgcgatgatggtgagccttcactcaacttgaaatctgcatcgttgtcatcttcagctggcagcaaggggctagatgaattacctcccacatttaaagaagcaaaccaaaccgtggatggctcagaagctgttgcggccaagtcagatgctcctgaaattggttgtcaggtacctggttcctctccattacatgacaaaggtagtgaatcctcggggtcgccagtgaaggacactcctgacgctggaatcagattgccaggatcagataactcagttatggcattttctgcactcccacttgttgatgaagcacctgggacaagcagcagagcagctgacacgcctgtgccatgtgatggcaatgagtcgaaagaggatgttccactagacaacgctgaaagcaccaggaaactggagccgaacggatga

SEQ ID NO: 75 - DNA, 798 bp, Zea mays
  source 1..798: mol_type = genomic DNA
atggatgatgcagcaggaaatattcttgcatcacttggccgtgtgcatctagttgatctcgtggcctctgagggcctgccttctgacaactataagatgtgtgtatcaactctgatgcagtcactgtctcagtattcgacagccatcattcagctgtcaccagctgatggtgccttgttgaggtcaggtttggaatcggctcgctttttcttccaccagcaagggtataattggaatgaggctgtccacacgaatgatccctgtgaatggcctggtattactgcaaaagatcccagcagtgcaaaggaattgccccgtcaggagcaacagcttgataagactttgcttaccctagttaagtcagacaggtcaggcctatatgttaaagacttacatggatgctggattcttgttgatggagatattggtccacacgatgttgttgtctatcctggccttgcgctttatcaggaaactgctggctatgtaaatccagctgtacataaaacagaggttggaaacatacatggatgtatgtttgggcgatgttctctgatgttcaagttcatgcctagatcagttgcaaggctgagtggttcagagatgagagctgctggccatggcgttgatgctcagtttcagatcattttgctgatcaactgtttcctaagactgaaccatcattacacacagaacaagattcagcccatgtacatgccgtgtaaacgctacacgatggcttacaatgtccgtttagtcagtcgtttatctcggttaaccaccgtttcgtctgtttaa

SEQ ID NO: 76 - DNA, 1020 bp, Zea mays
  source 1..1020: mol_type = genomic DNA
atggggaagctgctccctcgccacccttcccccctcctcctcctcgccgcggcgatctgcatctccagcagcagcctcgcggccgtccggggcgcgccggtgtaccgcgcggactacctggtggacgggaaccagctggtggacatgcagtaccacatgggccccgtcgtgtccgggacgccgaccaacctctacctcatctggtacgggcggtgggaggccgcggcgcaggccgtgctccgggacttcctcgcctcgctctcggccccggccccgttcccggccgtctccgactggtgggcgcgcacgccgcggatgtacgcggaccagacgggcgccaacgtgacgggcgccttcgccgtcgccggggagcactccgacgccgggtactcccacggcgcgtcgctccggcgcatcgacgtgcagtccgtgatccggtcggcggtgtacgcgtacccggacccgctgccgctggacccctacagcggggcgtacctggtgctgacgtcccccgacgtgcaggtggacgagttctgccgcgccgtctgcggcttccactacttcaccttcgcgtccgtggtcggggtcacggtgccctacgcctgggtcgggaacagcgccacgcagtgcccgggcaagtgcgcgtacccgttcgccgccgccgccgagtacggcgccgggggagcccagccccaggcggtgctgcggccgcccaacggcgacgctggggtggacgggatggtgatcgtgctggggcacgagctggcggagatgtccacgaacccgctggtgaacgcgtggtacgccggggacacgcccacggcgccgaccgagatcgcggacctgtgcctgggcgtgtacggtgatggcggcggcgcgggcggcctggtcgggaacgtgtcccgcgcgcccgatggcagcgcctacaacgtcaacggcgtcaacggccggcggttcctcgtgcagtggctctggaaccccgtccgcggcgcctgctacggccccaactccagcaactga

SEQ ID NO: 77 - DNA, 2568 bp, Zea mays
  source 1..2568: mol_type = genomic DNA
atgtccaccgccggggacccttcccgcctctccggcgagtcctcgccgtcgtcctccacgtcctccggctcctcctcccactcctctggcgccgccgatgccgccgccaccaacctcgccctgacagcaccaacctccgccctcgccgatgacacagacgccgatgcccccacctccccgcgcgtggggacgtactttgagaccgaggacgacgcgtacgagttctacaaggcctacgcggcccgtctcggcttcgtcgtccgcaagtccaacaagtccaagaactcacggcacaccgtcacccgccgcctcttcgtctgctccaagcagggcttccgccaggagcccaagaagccccaggacgaaaccgcaggctccggagtcgcgtcctcgccctcgctgtcgctggtgcctgcgcctcggtgcccggactcgcgcacaggctgcctagcgtcgctcaccatcaagctcattccttccgcaaacgccttccgtgttactgacttcgtcgccgatcacaaccacccgctcgcctcttcctcgcccgctgtgtcactagccttgctgtcgccgagctcttcacaccacagcattgtagcagtggccagcttgccggacccaagggatgggccgcgggccgatatgcattttgagacagaggaggatgcatatgtcttctataacagatatgccgaacatgtaggcttcagtgtacggcgctcctacaagaagcggaagcgagggatgatagtgtcgcgtatttttgtttgttctcgcgagggtgtcagtgaccgcaccaagcaagagggtggagccatagtcattgcaaatggtggtgcagggtcagcaggcacacctaggccaggtccgccgccaacacggaccggatgtcaggcaagaatggtgataaagatcaccccctgccgaacataccgtgtagcaaagttttttccagagcataaccatcctctggccaatccagacagcgtgcataagctgcggtctcataaaatgagagcccgtgcacatgagcttggagcaggggaaatgcatcgaaggaagcaagggaagggtgtgcagcttggggatgttggcgcagcattgcaatacttggaggagttgcaggtagaaaatccttcagtttattacgcggtaggagtggggcctgatgggaaatccgctgtgaatttcttctgggctgatgcaaaatcaataattgactacaggagttttggggatgttgtttgctttgatacaacatatgcattgaacatttatggacggccatttgcactctttgttggtgttgacaaccataagcagttacttgtgtttggcgcagcactgctctatgatgagagcattcagtcgctgaaatgggtatttgaggtgtttgctgatgccatgcatgctaggcacccacaaactattttgattgatgaacgtcctgagtgtgccattgcagcagcagaggtgtggcctggcaccaatcactgcacaggggtgtggcatatctaccataattcaaagaggcacttgaagcaggtgtttgaaagctcaaaaagtttcagcaatgctttgaaccattgtctctttgaatgtgaggatgagattgagttcttgtcagcatgggaaaagctagttgagaaacatgacattggtgagagtgagtggctcagcagactttacctggagaaagaaaaatgggctttaccttatcagagaaccatgttttctgctgatatactctcaacccttcgtaaggataacatgattaatgaactaagacgagacctcagtgagcaagaagatatcctgcagttcttcaggcgttatgagtctattctggaagagcatcgatcaaagaaattgcatgctgatgttgatggcagccaggttactctgcctatcccatcgttgcgaatgctaaaacaatcatcaaatgcttatacacctgaagctttcaagatgttccagggagagtttgaggcttacatgaattgcatgtccttcccctgcggtgtggttggcacgatctctgagtacaagattgtgcttgatgagaagccatcagagagttttgtcaaatttgatgcactagatggctcgaccacttgcagctgcaagaagtttgaagctgttgggattcaatgctgtcatgtactgaaagtgctggatctcaagaatatcaaagaacttccagaacaatatattttgaagagatggaggaaagatgctcgttctgttcaaattggggaggaacctacctatgggtctggcagtgtcatgcgatcaacctcagaagctcgattcagtaacatgtgtcggttggctagcttgattgcttcaagagctgccaaatccgaggatgtgatgtcatacatcgaaagtcagtcaaatgctattcagaaacatctggatgatattctacacacaagctatcctgatatgggaaatcacactgttgcttcaagtagtcaagcaatttcctttgtaggcacccaacatcctgaccattccacaccagtagtggttgcacagacaacaaatggcctgatggggctttga

SEQ ID NO: 78 - DNA, 1461 bp, Zea mays
  source 1..1461: mol_type = genomic DNA
atgtcgtccatctcgcggaatctccggcgatccctcgccccggaccgcctgcgccgcctctcccagcacccgcgagagtatgtgacgtcggagtgccaccgccccgtcgtcctccacaagcgcggccccgacatcctccacgacccctggttcaaccggggcacggcgttctcgatgaccgagagggaccgcctcggcctccgcggcctgctcccgcccaacgtcgtctcctcgcagcagcagatcgacagattcatgcttgatctgaaacggctgcagagatatgcaagagatggcccgtctgatacttatccattggcaaaatggcgcatccttaacagattgcatgacagaaatgaaacgatgtactacaaggtactaatagataacattgaggagtatgcacctatcgtttacacaccaaccgttggacttgtttgccaaaactacagtggtctgtttcgacgccctaggggaatgtactttagtgcagaagatcgtggggagatgatgtccatggtgtacaattggccagctgatcaggttgacatgatagtggtgactgatggaagcaggatattgggtcttggtgatcttggggtgcatggtattggcattgctattggaaaattggatttatatgttgctgctgctggaataaatcctcaaagggttcttcctgttatgattgatgtcggaacgaacaacgagaagctccttaaggatcctctttatcttgggctacaagagcaccgtcttgagggagatgaatatgtttcagttattgatgaattcatggaagctgtttttactcgctggcctaatgttattgtacagtttgaagatttccaaagtaaatgggcctttaggttgttgcagcgttacagaaagacttatcgcatgtttaatgatgatgttcagggaactgctggtgtcgcaatagcaggccttttaggagctgtacgggcacaagggaggccaatgatagattttccaaagcaaaggattgttgttgctggcgctggcagtgctggtattggagtggtgaatgctgcaagcagaaccatggcaagaatgttgggaaacaatgacgttgcttttgagagtgcgaggagtcaattctggatagtggatgcccatggcctaataacagaagaccgagaagatatcgatccagatgcacggccttttgctagaagaaagagtgagcttggtcaccagggcctaagtgagggggcaagcttagttgaagtggttaaaaaggtgaagcctgatgtcatacttggattatctgctgttggtggtttgttctcgaaggaggtaagaatagttgcatgttactttctgcttattctgaagcatttactccaatgctatagtattaatagtaattcatgcttactgggcagttacttttttcacccttctactggtttccttattgcaacttttttttcataa

SEQ ID NO: 79 - DNA, 627 bp, Zea mays
  source 1..627: mol_type = genomic DNA
atggcaccggtgtcggcgctcgccaagtacaagctcgtcttcctgggggaccagtccgtcggcaagaccagcatcatcacccgcttcatgtacgataagttcgacaacacttaccaggctacaattggtattgatttcctgtcaaagacaatgtaccttgaagatagaactgtgagactccaactctgggatacagctggtcaggaaaggttcaggagtttaattccaagctatatcagagactcttcagttgctgtcattgtattcgatgttgcaagcaggcagtctttcttaaatacatccaagtggatagatgaagttcgcactgagaggggcagtgatgttatcattgtgcttgttgggaacaaaactgaccttgttgacaagaggcaagtctcgatagaggaaggagaaggcaaggcaaaggaccttggtgtcatgtttattgaaaccagtgctaaagctgggtttaacattaaggcgctgttccgtaaaattgctgctgcccttcctggtatggagacactctcatcagcgaagcaggaagacatggttgatgtgaacttgaggtctggcaatgcaaactcgtcccagtctcaggctcaggctgggggatgcagttgttag

SEQ ID NO: 80 - DNA, 327 bp, Zea mays
  source 1..327: mol_type = genomic DNA
atgcagtatttggcatcatttgaatttggagaagaaatttccaatgtgtttggagatttgaaagatgacaatggatccgatactggcattaattttgataacattttttgggcaatagtccattttatgcatatacaattaaaggagcttctgaagtatagggatgcagcacaaatggttgatgttgaaaccacgcaagaagctgctatggcagagatccttgttcgatgtttaagatgcgcagagggaggagaagaatgtggaaaaaatgtcagggtggctaccaagtgcaatgatatggaatctgcaaagctatggccagtctga

SEQ ID NO: 81 - DNA, 3828 bp, Zea mays
  source 1..3828: mol_type = genomic DNA
atggcgacgcagtcgcactcctcctccgctgccgcgatagacatgtatgagacggcctctcagccggacgcctccgttgccggcgacgcctacaccttccttgagttcaacactcagggtgacgaattcgactaccccgactttccggagctttctcaaccgccgccccgatcgacgccgctgacgtcttccatccccggcgctggcgcgtcgtcctcgtcgtggcctgccccgccgccgccgccctccgacgcggcctccgctgaacccgatctcacgtctctggacgtccccaccccgccggcctcctcctcctcgccgtcgccgcggtctgcctccaaggcgcgctcgttggctgcggcagatgggctcgcctccggggtagccgcactcagcttcgaggatcctgttggagccggcgccggagaggacgggtacgactacggcaagggagacttcgtggaacacgcctgccggtactgtgggatccacaatcctgcatgcgtggcgaggtgcaacgtgccctcctgccgcaagtggttctgtaactcgcgggggaacacctctggctcccacattgtcaaccacctggttagggcaaagcataaggaagtgtgccttcacaaggatagccctttgggagaaacaatccttgaatgctacaactgtggctgtcggaatgtatttcttcttgggtttatctcagcaaaattggagaatgttgtcgttcttttatgcagagagccgtgtttaagtgttaatgcactgaaggatatgaattgggatctcagccagtggtgccctcttattgatgacaggtgtttcttgtcatggcttgtaaaggcaagggtaccttcagagcaggagcagctaagagctcggcaaattagtgcacagcaaattaacaaagtggaagagctttggaagacaaatcctgatgcatctcttgaagatcttgaaaagcctggcgtagatgatgaacctcagccagttgttttgaagtatgaagatgcttaccagtaccaaaatgtttttgcgccactgataaaacttgaggctgattatgacaagatgacgaaagagtcacagagcaaggataatgtaactgtccggtgggatattgggttgaacaagaagcgtgtagcttactttgtctttccaaaggaagacaatgagttgaggcttgtgcctggagatgagctacgcttgagatatcctggtgatagctcacatcccacatggcaatctgtgggacatgtgattaagctcactgcacaagaggaggtggcacttgagcttcgtgcaagtcagggggtgcctactgagctaaacgttggattcagtgttgatttcgtttggaagagtaccagttttgataggatgcaaggagcaatgaaaacttttgctgtggacgagacaagtgtcagtggatacatataccaccaccttttgggacatgaagttgagcatcaaataatacgtaacaccctaccaagacgctttggtgcacctggacttccagaactaaatgcttcccaggttttagcagttaaaagtgtcctgcagaagccagttagtttgattcaaggtccacctggcactggaaaaactgtcacatctggtgcaattgtgtatcacatggccaaacaaggtcaaggacaggttcttgtatgtgccccaagtaatgttgctgttgatcagctggcagagaagataagctcaaccggtctcaaggttgttaggctgtgtgcaaaatctagagaagctgtttcgtctcctgttgagcatctcacacttcattatcaggttaggcaccttgatacctctgaaaagagtgaatttcacaagttacagcaactgaaagatgaacaaggtgaattgtccagcagtgatgagaagaagtacaaggcactaaagcgtgccactgaaagagagatactgcagagtgctgatgtcatttgttgcacctgtgtcggtgctggagatcctcgtctttcaaacttccgtttccgacaagttcttattgatgagtccacacaggcaacagagcctgaatgtcttattcctttggtgcttggtgtcaagcaggttgtccttgtcggagatcattgccaattgggtcctgtcatcatgtgcaaaaaagcagcccgtgcaggattagcacaatctctctttgaacgccttgttatccttggagtgaagccattcaggctacaggtccagtatagaatgcatccatgcctctcagagtttccatccaactgcttttatgaaggcacactgcaaaatggagtgactgttaatgagaggcattcgtctggaattgattttccttggcctgttccaaatcgcccaatgttcttctatgtgcagatgggacaagaagaaatcagtgctagtgggacatcgtatctcaatagaactgaagctgcaaatgttgaaaaaattgtaacaacttttttaagaagtggtgttgtaccaagccagatcggagttatcacaccttatgaagggcaaagggcatacattgtaaattacatgtcaaggaatggctctcttcgccaacaactttacaaggaaatcgaggtcgctagtgtagactcatttcagggtagagagaaagactacattattctatcttgtgttagaagcaatgagcaccagggaattgggtttctcaatgacccgcgcaggttaaacgttgcgttaactcgtgcacgttatggcatagttattcttggtaatccaaaagtactaagcaagcagccactttggaatagcttgttgacacattacaaggagcatgagtgcttggtagaaggacctctgaacaacttaaagcagagcatggtgcagtttcagaagcccaaaaagatatataatgatcggagattgttactgggcggtgctcaaggcatgcatggagctaacttcggggctggggcctcaagccaagcagcagataagagaagtggcagggggaaagcagggcaatcatttgtttcttatggccctccaaatggggtgcacaagcctggtgtgcacccagcaggttatgctgtacctcgtatgccttttcctccatttcctggggctccacattctcaaccatatgcaattccgactcggggtttgcatgggcccattggagctgttccaccggtgcatcaacctggaagtagaagttttggggcacctcgttcaaatactggtgggcctattggtggccaccttgctcatcaacagaattcccagcaagctatgggtggcattgggtcaaattttaactacgctggcatggagaattcgagtagtcagccttctggtgtagcccaaatgtcccaatcgggattaatgacacagatgcctgttcaaggactaagtcaaacattccgtgatggtttttccattggtgggatgtctcaggacttcattgtagatgattttaagagccaaggatcacatgtgccatacaacattgcagaattttctacccaggcttctcaaggtggttatggtgttgagtttactcaagcgccccaatctggatactctgggaattatatgaaccaaaacacacatcctggatactcccatattggtacaacaaatgacattgtatctcaggatcacatggcccatggatctcacggaatgtttacgcaagtggggtataatgatccgtcgcaagatgagtcatctcagattcattatggaatggctgctgctggtcccctacattcgcagagcatgatgaaccccttatactctcaatcctatgctcactacaacacccagccacattctcttcaacctcctccccagtga

SEQ ID NO: 82 - DNA, 612 bp, Zea mays
  source 1..612: mol_type = genomic DNA
atgctgcgccacgccgcccgccgtctcgtctcccgcaccgccgcggcgcccaccgcccgccgggcgctcgtcacggcagaggtgcctgcggaggccgccgaggactccacgtttgtggaggcgtggaagaaggtcgcccccaacatcgagcccccaacgacgcccctcttcctcatgcagccgcgcccgcccacgccggctaccatccccactaagctcaccgtcaacttcgtcctcccctacaagtccgagatcgccaacaaggaggtggacatggtaattgtaccagctacaactggtcagatgggcgttttgccaggccatgttgccacgattgcagagctcaagcctggtgttctctcagtacatgaggggaacgatgtgaccaagtactttgtgagcagtggatttgctttcgtccatgcaaactccattgctgatattgtggctgttgaggctgtccctgtggatcagatcgacccagcttttgttcagaagggtctcgctgaattcactgccaagcttggttcggcctctactgacctggagaaggctgaggctcagatcggtgtcgatgttcacagtgcgctcaatgctgcattgaccggttaa

SEQ ID NO: 83 - DNA, 822 bp, Zea mays
  source 1..822: mol_type = genomic DNA
atgtataaacaggggggagcggggggaggggctgggctggaccggaagcggatcaacgacgcgctggataagcatctcgagaaggcggtcgcgtcgccgtccacgtcgagggggtcggcgggcggcgggcgcgaccaccaccgcctcgtcgtgccgtcctccgtgtcgtccatccccaagggccgctgctccgaaggggagtctgattctgatagtgaagcatcggatgttagcggctctgatggagaagacacctcatggatttcatggtattgcaacttaagagggaatgagttcttctgtgaagtcgacgatgattatattcaagacgacttcaacctttgtggtctcagcagccaagttccttattatgattatgctctcgatctcattttagatatcgagtcatctcatggtgatatgttcactgaggaacaaaatgagctcgtagaatcagctgcagagatgctttatggtctgatacatgcacgatacattctgaccagtaaaggactggctgcaatgctggagaagtataagaactatgactttgggcggtgcccgcgtgtttactgctgtggtcaaccatgcctgccagttggacaatcagacatccatcgatctagtaccgtgaagatatactgccccaagtgtgaagacatctactacccacggtccaagtaccaaggcaacatcgacggcgcatactttgggacaacgttccctcatctgttcctgatgacatacgagcacctcaagccacagaagctctcgcagcgctatgttcctcgtgtctttggcttcaagctccacaagccatga

SEQ ID NO: 84 - DNA, 1062 bp, Zea mays
  source 1..1062: mol_type = genomic DNA
atgacgcgcatccaccgcctccgccccctcttcctcttcgttctcttggcgtcgctgcaatgcggtaatgccgccaaaaccctagccgggctctccgccgccactgtggcgtccacactcgagtctccactggaccagtcctcgctcttgtttcactcactacacggtctgtcacttgatccgattgcaggtgcggcgacggcggcggctggatacggaggtggcgccgaggggttctgctcggcggagccgtccagcaagtgctccggggatcagccgctctactggaaggtcacccatcccaccctcgcgcccgcgcacctccaagatttacctggcttcacacgtagtgttttcaaaagggatcatgctttaataacaccagagagtcatgtattcagtcctttgcctgattggatcaatacagtaggagcatatttgatcagtccagctattggtgcacacttcattatgtatctagcaaacatgcaagatggttcaaaatcagccctaccaccgaaagatgttgaaaggtttgtttttgtactacaagggagcatttcattgactgtggggactggaactatccattctctgctggtggattcttatggatatcttcctgcaaacacaaagcattctatgatctcagatgaaccaaccactcttgtaatatttgagaggaggtacaatgccattgaggattcccatcctgatctaattgtgggttcaacagataagcaaccccttctcgaaaccccgggagaggtatttcaactaaggaagctgctaccgacttctctgtcctatgactttaatatccatataatggactttcaaccaggcgaatatctcaatgtaaaggaggttcattataatcaacatggacttcttcttttagaggggcaaggaatataccgactgggagagagttggtatcccgtgcaagctggggataccatttggatggcaccgtttgttcctcagtggtatgctgcccttggtaagaccaggacgagatacttgctgtacaaagatgtcaataggaatccattgatatga

SEQ ID NO: 85 - DNA, 1209 bp, Zea mays
  source 1..1209: mol_type = genomic DNA
atgggaagcgcttgggccaacgtccgcgagatctgccgcgcacagcgtgccgacggccccgcagccgtgctcgccatcggcacggccaacccggcgaactgcgtgccccaggacgagttccccgacttctacttccgcgccaccaagagcgaccacctcactggcctcaaggaaaagttcaagagagtttgccagaagctgggcgtgcagaagcgctacctgcaccacaccgaggagctgctgagcgcgcacccggagttcctggaccgctcctcgccgtccctcgacgcgcggctggacatcgtcaagaccgccgtcccggagctcgccgcgcaggcctgcaggaaggccatcgccgagtggggccgcccggccgccgacgtcacgcacctcgtcgtcgccaccaactccggcgcgcacatcccgggcgtcgacttccagctggtctcgctcctgggcctccgcccgaccgtgcgccgcaccatgctctacctcaacggctgctccgcgggcgccgccgcgctgcgcctcgccagggacctggccgagaacaaccgcggcgcgcgcgtgctcgtcgcctgcgccgagatcaccgtcctgctctttgacgggcccgaggagggctgtttccagacgctcgtcaaccagggcctgttcggcgacggcgcgggagccgtcgtcgtcggcgccgacccggcggccccggccgagcgcccgctgttcgagatcgtgtccgccgcgcagaccgtcataccggactccgagggcgtcatcaccatgcacctcaccaagggtggctacggcggcaacatctccaccaggcaggtccccgtactcatcggcgaccacatcgagcgctgtctcacggacgcttttgcgccgctcggcgtcggcgccggatggaacgacctgttctgggacgtgcacccgggctcgtcggcgatcctggaccaggtcgacgccgtgctcaagctcaagcctgagaagctggcggcgagcagacgtgtcctcagcgagtacgggaacatgttcggcgtcacggtgatcttcgtgctcgacgagctgcgccgccggatgacgaatggggaagaggagggggcgcctgcagagtggggggtcatggtggcgtttggacccgggctcaccgtcgagacgatggtgctccaccgatcaggcaccccagccgagacgaaactaggtgaggcatga

SEQ ID NO: 86 - DNA, 333 bp, Zea mays
  source 1..333: mol_type = genomic DNA
atggcggaggccatcgttggtccgctggtggggaagctccaggagatggcggtgagcgaggcgaaggctctagtggcagtgaacgacgacatccgcggcctccgggaccggctcatgtggatgcaggccttcttgcggcatgccgacccgcggcgacgcgacacctccgacgagctcatcagggtgtggctcaagcagacccgcgatgttgccttcgacgccgaggacgccattgacgactactccctcaaggtcgacctctcaagccaagaacctgaagggagactgggagtacacatcgccactgacaaatcaaacatcctttctttgtaa

SEQ ID NO: 87 - DNA, 3078 bp, Zea mays
  source 1..3078: mol_type = genomic DNA
atggcggaggccatcgttggtccgctggtggggaagctccaggagatggcggtgagcgaggcgaaggctctagtggcagtgaacgacgacatccgcggcctccgggaccggctcatgtggatgcaggccttcttgcggcatgccgacccgcggcgacgcgacacctccgacgagctcatcagggtgtggctcaagcagacccgcgatgttgccttcgacgccgaggacgccattgacgactactccctcaaggtcgacctctcaagatatccaaattggagccagtccattgtttgttttttctctggattcactacacaagtaagcattcgacacaatctatcaagaaagatcgcagagatcaacttaaggcttgaagacatcattaacaataaggagaaatacaagattgaagctgcacccaatgattttccattggtgacatggaaaccttctacggacatctctactgtggcaaaaagtttggatgatgtccaaccatcttcgatcaaacgggaggagctggggaagcagcttgaagaagcacttttctgtgacaataagaaccgacttgttatatgtgtgacaggggaaggtggtgtgggcaaggcaacactaatcagagaactatatgaaaagccaacaacaaagtcaaagcagtttgaatgtcaagcttgggttagcttcccaccgtatttaagttcttcaagcatcctacagttaatccatcaggaattggaggaaacagatacttggtgtcccagaaaacaagtagacaagaaactgcacgaaatattggataaggaccggttcttgctcgtcatagatggagatgttagcaatagtgattggaatgccatccttgccgcattaccagaaaagaacaacggtagtaggattgtccgtatcatgcaaggtatacataagcagccacgtggtattgctgcaaaacactggattgagctgaaatgttttgaaacagaaaaaaccactagcctattcagccaaagggtgtgcatggaagaaaaaataaatattgaaaactttgatgaagttctgcatggtataaccaaaggccttcctcttgccattgttcttctgtctggccttgtacgaacaaaggagtacccaaatgagtggcaggcagtacttgaacacctcaaatccaagaaatcaaaaaggcttgatagcatacttgccatgtcttttgacgatctcccacatgacctaaaatcatgcttcctctactttgctgcattgcctatgaacacgcctgttgaggctcgcaaattggtatgcatgtggatggcagaggggtttctaagaccaaaagatgggagaacaatggagaaagttggaagaatctatttgaaggagttggttatgaggcatctagttaagttagtgaagacagacaatgccggtggtggtgatgaatttgtggccgtccaccataaggttcatgaatttttgcagcacgaagctcaagaagctaactttgtggatatccacaatggtgacgatatcccttctttggcaactactcgtcgcctatctctacaaaattatacagataaatatgctgctctgtccagctctttgccaaaactacggtctatcttgtccaatttccaggaagaggttgaagatgacgaggaagatgaagataatgaggaaagtgaagatgacgaggaagatgaagacagtgaggaaagtgaagatgacgaggaagattcagaaaaggaaggagacatggaggcagggcataaggtagaaggtgatggcgatggagaggtagaggtagaagaagatgaaaatactgagattgtgttatgtggagacaatgaaaacaatggggaggttgaggggtcatttgaaattactgaggaaggaggggaaaatgaaacaagtgaagaagcaaaacaggatgtgatgcccagtacaacaatgccattattaccaaacttgcttcagtgttgggtgacgccactatcaggcttgcttcgttgttgtggggaacatggcagccctcaagagagcaccaactcatacataaaagaaatgttgcaagtatccaagtttcttcgtgtcatcaatctacaaggcatcgagatcggtgagagcttaccaacaactattgggaatgtagcacaccttcagtacctcggtgtcactgcttgttccttgaaatatatcccatcgacaattgaaaaccttaaaaatcttcagacgttggatgttcgagacacgtttgtttataaacttccagaagcattttggagtataacgacactgcggcatgtatttggtgagggtcttttcttgcctaagcaggtgggtgacttgaaacacctgcagacacttgaaagcatagatcctgataaggataatggttgggacagcagcacttttgagaaaatggctcaccttcagtcccttcatgtttgggattctaaggagaatggagttaatgctgaggctctatctgctgttattgataaagctaatttcctcgaacacctagataccctcactcttgatgttcatgacatcccattgagtgtgttcaccagttcttccctgaggcgtcttcgtaccctaaacttagacggcaagttggacatgttggtgctgcaatctaaagaaaaggagtcaaaatttcatttccccaaccttagcttcctatcattggcaaacacagaggtgtcgcagaactttatcaacaagcttgggaagctgccttcgctcaccactctaatattggatgtaaaatcctacaaggatgatcaagatcaacttgccttccaagctggtggatttacaagccttacaaagctgatactttctgatctggaaattgtgaaactcgagataaagaaatatgcacttccagaactcacagatttggaggttgagttgtactacgacgaaatcgaaatcgaggtatatggcgagcatgagtttgtgaagaaaattgaacaagtggatgaatacctctatggccgtatcactcaagttccggttgctccaaagaaaattggtcagcttcctagcatgaagcaacgcgtggctagctga

SEQ ID NO: 88 - DNA, 1323 bp, Zea mays
  source 1..1323: mol_type = genomic DNA
atgcggtggagatctcccgccgtgctgcggctgcagctgtggctgctcgccgtctccgcgtcgttggctgccctcggtgttctcgcggctgatcttagcaaagaaccatttactatccgtataagctgtggaagctttgacgatgtccggacggagcctacgaacacattgtggtacagagactttggttatactggaggcaggttcgctaatgccacccggccaagctttatcgttcctcctcttaaaacgcttcgatatttcccgctatctgatggccctgaaaattgctacaacatcaacaatgttcccaatggccactatcaagttagacttttctttgcgttactggacaaccctaatcttgacagtgagccgattttcgatgtttctgttgagggtactctgttcagctctttgcttctgggctggagcagcgacgatgagaagacatttgcagaagctttggtttttgtccaggactcaagcttatcagtttgcttccatagtacagggcatggtgatccatcgattctttctattgaagttctacaaatagatgacgatgcctataactttggtccaccacgggaaaaggggacagtgcttagagcagtcaaaaggctgaaatgtggctcaggaaagcctgcttttgatgaagatctaaatgggattcactggggtggggacagattctggttaggattgcaaactttatcatccagttctgatgatcaatcaatatcatccgagaatgttatagcagagacattgcttgcaccaaatttttaccctcaaggcatataccaatcagctatcgtgggcactgataggcaaccgagtttgtctttcgaaatggatgttaccccaaacaagaactattcagtatggcttcactttgcagagattgataatggggtaactgcagaagaacaaagagtatttgatgtactcatcaatggagacactgcttttaaagatattgatataattcgcatgacaggagagcgttttactgcacttgtcctgaataaaactgttgctgtcagtggaataacactaaaaatcatcttgcaacctgtgaaaggaacccgtgccattattaatgccattgaggtgtttgagataatcccagctgaaaagaagactttgcctcaggaagtgagtgcattgcgaacgttgaagggttcacttggtcttcctcttcgatttggctggaatggtgacccctgtgttcctcagcagcatccatggagtggagttgattgccagtttgacagcaccaaagggaactggatcattgatggattgtatgttctctag

SEQ ID NO: 89 - DNA, 1677 bp, Zea mays
  source 1..1677: mol_type = genomic DNA
atggggcccgccgtccgcagtagcgtcgtgatcagggccactgtggtagggctcgtgctggttggcatcgccctggccctgccgctggccgcggcgctgcggccgctccgggagcacgtcgcgtccatcggagccgcagcctccgctggttcgtggggcgatgagcatgccttctacaaaagggatgagaacgatataggcccatattcatggaatattactggaacatataaagggagctggacttttgctggtgccacaaatggttcgtctagatttcttgagtttgtggaatctaaaggtgattcagttttggaattgttgagcactccaacaaagataagcggggtgcattatgttcagggttcagttacattccatgatgtcattgacaatgcccacaatggtggagtggctcaaataaggttagaaggagtttacatatggccttttagacaacttcgcatggtagcaaacagtggtgcagatggtgaaccactccaggaagaggattatttcttgtcaaatccataccatttattgcggattttctcctctcaagtgtttcaagaatcttctgaagagaagaaccgaaggaagaattctctcacatatgacatggagaaacattgcaacacagaaatagctgctaaggtggttcgtgtgtcatctaacccaaatgatggagaacacgagaagtaccgtttggagggtttgatggagagtccagccgtcgatgatgatggggaatgcttctctcctatcttattgaatgcaacttctttgaatgttgaagtttattacaacaaagcagtcaactacacactgatgggagctgctaaggtttctattttaatgattggccaacaagctatcatggatgcatatctctgtctacttcacctgactgctgggatattggttgagtcactcttcaatgccttcgcaacggctgcattcttcaaatttgttgtattttctatttttgaaatgaggtaccttctcgccatatggaaagcaagtaggcctttaaacagtggagaaggttgggagataatgaggcgagagctgtctgttctttacagccgcttttatggcattctgttgggaggaatccttctaatgtatgagctgcacaatttcttacgcccacttctcctcctgatgtactccttttgggtacctcaaattgtcacaaatgtcatcagggatacaagaaaacctctgcacccacaatatattttaggcatgacagttacacggcttgctattccattgtatatatttggttgccctagtaatttcatgcgtatcgagcccgacaagaaatggtccatagctgttacagtattcatgggtatccaagcagcagtgcttctgcttcagcactatcttggttctcgctgctttattcctcgccagatactgcctgagaaatactgctaccataggaaggtagaggacagcacaaatcagcccatcgattgtgttatttgcatgactacaatcgaccttacccaaagaacaagtgaatacatggtgggggaactgatgtacaataagtatttcggcccagcgttttggtctgagctttgtgcaaagtttgttaacgccgtgaaagagttcgagatgtgcattcttgatttggtggacacattagtagcgtgtttggtttga

SEQ ID NO: 90 - DNA, 684 bp, Zea mays
  source 1..684: mol_type = genomic DNA
atggacaatgaaagggaacaatcgttgggggatttggagaaagagttggaggcaaaagtgtcggacacccagtgcgctgctgctgaaattcaggatctattgaaacaagtggatgctcagccggtgaatgtgaaggatgtggatagaatgcgtagggagatgcaggctattgaagatgacattgccaacaccgagaaggggaaagcagcactggaggataagttctgggaagttgaagctaagctggttaccaagcttgaggagcttgagaggcatgctcatcagttcaaccaggccctcaaacagttgaaacctactgttgcttttcagtacatgatagattcaaaaggatcttctcctgctgagatgctaggtactggttacaaaacagtactaaaacctgcacttctggctcatgctgaggagaacaaaaggatctgtctctcaaatcttgaaaacttaaatgatctacagaagcagctgcaaggaaatgccaaggttctagaggaggaaaggaataatattttcagtctccaggcaaaaaatgacgatctatgttgtgcagtagtgggaacttttttaggttctggaagggatgtacttccacgaactgatcgagatctcacacggcgaccgacggcggtaaatgaatattcttgtggtgtagacacactcaacatttga

SEQ ID NO: 91 - DNA, 1452 bp, Zea mays
  source 1..1452: mol_type = genomic DNA
atgtcttcggccgccccgctgctaggcgacgaggccggcggtggcgagcccgcgccgcggacgccgccgccgccgtcctcgtgggtggggcgcgtggtggacgcggccgaggcgcgcgcgcagctggggttcgcggtgccgatggtgctgaccagcatggcctactacggcatcccgctggtgtcggtgatgttctcgggccagctcggcgacgtccacctcgccggcgccacgctcggcaactcctgggccaccgtcaccggctacgcgttcgtgaccggtctgagcggcgccctggagacgctgtgcgggcaggcgtacggcgcggggctgcaccgcatgctgggcctctacctgcagtcgtcgctcatcatgtcggccgccgcgtcggcgctcgtctccgcgctgtggtggttcacggagccggtgctcctgctcctgcggcaggagcccgaggcgtcccgcgccgcggcggcgttcgtccgcgcgcaggcgcctggcctcttcgccttctccttcgtgcagtgcctgctccgctacctgcagacgcagtccgtcgtgctcccgctcgtcgcctgctccgtggccccgttcgcgctccacgtcgcgctggcgcacctcatggtcaacgtgctcggcctgggcctcacgggggcggccgccgccgtctcggccacgttctgggtctcctgcctcatgctgctcgcctacgtgctgcgctccgacgccttcagcgacacgtggaggggcttctctgccgacgccttcaagtacgtgctgcccaccgtgaagctcgccacgccgtccgccgtcatggtctgcttggagtactgggcattcgagctcctggtcctgatcgccggattgcttcccaactccacagtgagcacgtctctcatcgccatgtgctccagcacggaggcgatcgcgtacatgatcacgttcggcttcagcgccgccgtgagcacgcgggtgtcgaacgagatcggcgcggggaacgtggagagggccaagaacgcggtgtcggtgacgatgaagctgtcggtgttgctggccgtcaccttcgtgctgctgctggcgttcggccacggcctgtgggcgcgcctcttcagcgggagcgccaccatcgcgtcggagttcgcggccatcacgccgctcatggtgatctccatcgtgctggactccgtgcagggcgtgctgtcgggagtggcgaggggctgcgggtggcagcacctggcggcggtcaccaacctggtggcgttctacatcgtcggcatgccgctggccatcctgttcgccttcaagctcgacctgcgcaccaggggcctgtgggcggggctcatctgcggcctcacgtgccaggcctgctcgctgctggtcatcaccgtccgcaccaagtggtccaagctcgccgatgccatgaaggaggagaaggccaactacgtcgcgtag

SEQ ID NO: 92 - DNA, 1341 bp, Zea mays
  source 1..1341: mol_type = genomic DNA
atggatgggcaaggcacaaagcacaggattttgatggtgtccgactttttctttccaaactttggtggggtggaaagccatatctactacctgtcacaatgtctacttaagcttggccataaggttgttgtcatgacacatgcatatggaaacagatctggagtacgatatgtcacaaatggtttaaaggtctattatgtaccatggaggccattcctgatgcagaatacacttcctacactatccttgacgtttccaattgtaaggacaattattattcgagagaagatttctgtcgtgcatggacatcaagctttttcaacactgtgtcatgaagcattaatgcatgctaggacaatggggtacaaagttgtcttcacagatcactcactttatggttttgctgatgctggaagcattcacatgaataaggttctgcagtttactcttgcagatattgatcaggcaatatgtgtctcacatacaagcaaagagaacactgtcttgaggtctgggatatctccagagaaggttttcatggttcctaatgcagttgacactgcaatgtttactccctctcccaagcgcttaaactgtgatgaaatagttattgttgtggtcagtaggttagtataccgaaaaggcgccgaccttcttgttgaagtcattccagaagtgtgtcggctatttccaaaggttcactttattgttggtggtgatggtccaaaacgtgtgcgccttgaagaaatgagggagaagttctcccttcaggatagagttgagatgttaggagctgtacctcatgctcaggtgcgatcggttctgatatctggacatatatttttgaacagttcactcacagaggcattttgcatagccattctagaagcagctagctgtggactgttgacagtgagcactagagtcggtggtgtccctgaggttcttccagatgacatgatagtacttgcagaaccagctcctggagatatggtaagagctgtcaagaaagccatcgacatgctacctggaatagacccccagattatgcatcttcggatgaaagaactttatagttgggacgatgttgccaaaagaactgagattgtttatgatcgtgcaatggaatctccaacaacaaatttactagatcgccttccccggtatcttacttgtggatcttgggctggaaaactgttttgcctagttatgatcatcaattacctactatggcgccttttagaatttctacagcctgctgtaggtattgaagaagtcccagatataggcccactacatgctcatttaggttcaaggaatgatgaggctcaagagaagtga

SEQ ID NO: 93 - DNA, 1431 bp, Zea mays
  source 1..1431: mol_type = genomic DNA
atggcagcccctaccgtcgtcttactgcccgtgtggggtgctggccacctcatgtccatgctcgacgccggcaagcggctgctcacccgcgggggtcgcgcgctctcgctcaccgtgctcgtcatgcgagcgcccacggagcagctcgcggccgatctcgacgcccacatacgcagggaggaggcatcgggcctcgacgtccgcttcgtccgcctccccgccgttcagcccccgacggacttccacggcatcgaggagttcatctccaggctggtacagctgcacgcgccccacgtcagggcggctatctccagcctggcttccccggtggcagcggtcgttatggacttcttctgcacggcgctgctcgacgtgacccgcgagctcgccgtgcccgcgtacgtctacttcactgccagcgccgggatgctcgcgttcttcctccgcctcccgtcgctccacgaggaggtgacggtccagttcgaggagatggagggcgcggtggatgtgcccgggctgccgcccgtgccgccgtcttccctgccggtgccagtgatggacaagaaccacccgaattacacgtggttcatgtaccacggcaggcgcttcgccgaggccgacggcatcatcgtcaacacggctgccgagctcgagcagagcgtgcttgctgccatcgccgacgggcggtgcacgccgggtgtccgtgccccgacggtctatccaattggcccggttatctcgttcagccccccgcccactaacacagaacatccgcacgagtgcgtgcggtggctcgacacgcagcccgcggcctccgtggtgctcctctgcttcgggagccaagggttctccgcggcgccacaggcgcacgagatcgcgcacggcctggagcgtagcggccaccgcttcctctgggtgctgcgcggcccaccggcacccggcgagcggcacccgtcggacgcgaacctctccgagctgctccccgatggattcctcgaaaggaccaagggcagaggcctggtgtggccgacgaaggcgccgcagaaagagatactagcccacgccgccgttggtggtttcgtgacacacggcggctggaactcggtgctcgagagcctgtggttcggggtgccgatggcaccgtggccgctgtacgccgagcagcacctgaacgcgttcacgctggtggcctatgtaggcgtcgccgtggcgatgaaggtggacaggaagaggaacaatttcgtggaggcctcggagctggagcgagcggtgaaggagctgatgggcggtggcgaggaagggaggaaggcgagggagaaggccatggagatgagggatgcctgcaggaacgccgtggaggagggtggctcatcctactcttcactacgaaggctatcagagaagatatgtaaagtcgacaagaacctatga

SEQ ID NO: 94 - DNA, 2889 bp, Zea mays
  source 1..2889: mol_type = genomic DNA
atggcagcccctaccgtcgtcttactgcccgtgtggggtgctggccacctcatgtccatgctcgacgccggcaagcggctgctcacccgcgggggtcgcgcgctctcgctcaccgtgctcgtcatgcgagcgcccacggagcagctcgcggccgatctcgacgcccacatacgcagggaggaggcatcgggcctcgacgtccgcttcgtccgcctccccgccgttcagcccccgacggacttccacggcatcgaggagttcatctccaggctggtacagctgcacgcgccccacgtcagggcggctatctccagcctggcttccccggtggcagcggtcgttatggacttcttctgcacggcgctgctcgacgtgacccgcgagctcgccgtgcccgcgtacgtctacttcactgccagcgccgggatgctcgcgttcttcctccgcctcccgtcgctccacgaggaggtgacggtccagttcgaggagatggagggcgcggtggatgtgcccgggctgccgcccgtgccgccgtcttccctgccggtgccagtgatggacaagaaccacccgaattacacgtggttcatgtaccacggcaggcgcttcgccgaggccgacggcatcatcgtcaacacggctgccgagctcgagcagagcgtgcttgctgccatcgccgacgggcggtgcacgccgggtgtccgtgccccgacggtctatccaattggcccggttatctcgttcagccccccgcccactaacacagaacatccgcacgagtgcgtgcggtggctcgacacgcagcccgcggcctccgtggtgctcctctgcttcgggagccaagggttctccgcggcgccacaggcgcacgagatcgcgcacggcctggagcgtagcggccaccgcttcctctgggtgctgcgcggcccaccggcacccggcgagcggcacccgtcggacgcgaacctctccgagctgctccccgatggattcctcgaaaggaccaagggcagaggcctggtgtggccgacgaaggcgccgcagaaagagatactagcccacgccgccgttggtggtttcgtgacacacggcggctggaactcggtgctcgagagcctgtggttcggggtgccgatggcaccgtggccgctgtacgccgagcagcacctgaacgcgttcacgctggtggcctatgtaggcgtcgccgtggcgatgaaggtggacaggaagaggaacaatttcgtggaggcctcggagctggagcgagcggtgaaggagctgatgggcggtggcgaggaagggaggaaggcgagggagaaggccatggagatgagggatgcctgcaggaacgccgtggaggagggtggctcatcctactcttcactacgaaggctatcagagaagatatcgtggagggaaagattagacgccgacatgccgagcccaaccatcgtgctcctcccgatgtgggctacaggccacttctcctcgatgctcgaagccggcaagaggctgctgttctgcagcgccggccgcactgcagcatacagcctcactgttctcgtgacgcccccgccgatggccgcgtcgtccgaggccagcagcagccagcaggcacactgtgagatgatggccaccgtcgacggcatcgttttccaccacctccccgccatcgagcaccgcaccgacctcgcccacccgtcggagtacatccggctctacgcgccgcaagtcaaggaggccatcgcgggtttggcggccccggtggcagcggtcgtcgtcgacttcttcgggaccccgctcctcgacgtcgcccacgacctcgccgtgccagcctacgtgtacttcgcgtccaccggcgccatgctcgcgctcatgctgcgtttaccgggcattcaggaggagctggcttccaggttgcgggaggagggaggggtcgttgacgtgccagggatgcctccggtgccggtggcctctatgccgtcaccggacgtgaacgactacacgtggttcgcgtactacggccgccgcttcctggaaaccagaggcatcatcgctaacacggcagccgagctggagccaggcgttcttgcgtccatcgccggcggcaggtgcacgcctggcgggcgtgctccaacggtgtacccaatcggcccggtgctctcgccgaagccgcgcgtcgtcgtcgatgctcggtcgtcgtcgtctgcgcaagagtgcatccggtggctcgacgcgcagccgccggcgtccgtcgtgttcctctgcttcgggagcatggggtggatgaacgcggagcaggcgcgcgaggtggccgccggcctcgagcgcagcgggcacaggttcctgtgggtgctgcgcggcccaccggccggcagcggctcggggcacccgacggacgcaaacctcggcgacctgcttcccgacgggttcctggagaggacgaaggcccagggggttgtgtggcctggttgggcaccgcagctggagatcctcgcccacgccgccgtgggtggcttcgttacccactgtggctggaactcggtgctggagagcctgtggcacggcgtgccgatggcgccgtggccgctgtacgccgagcagccgcttaacgcgttcgagctcgtcgcgtgcatgggtgtcgctgttgaccttagggtcgtcggcacgggaagggccagctccgtggtggaggcagcggaactggagcgagctgtacggagcttgatgggcgggtcagaggaagggaggaaggcgaaggagaaggccaggaagatgaaggcggcatgccggaaggccgcggagaagggcggatcggcgtatgctgcgttgcaggcagtggtgcaggatatgctagaaagccatgtacccggcacggcacccaaataa

SEQ ID NO: 95 - DNA, 621 bp, Zea mays
  source 1..621: mol_type = genomic DNA
atgccggactcctcctcctccgccggcacaggcggcatggccccctcgagctcggcggcggcgtccattgccgcgctcgcggccgccgcggcgtccggcgacggctcggacgactccggggcggccaacaacaacggcgcgctgccgctgccgctgccgccgccgcacggggtggaggacaacaacagtcccgcggagctggcgggcgcgggtggcacctcccgggcggtggcgaggagggtcgcggcggccggcgcctccccggcggcggcggcgaggaggggcgccgccgtgaacgcccaggcggccggcggcgccgggccgagctgccaggtggagcggtgcagcgccgacatgggcgtggagaagcggtacaaccggaggcacaaggtgtgcgacgcgcaccgcaaggcgtccgtcgtcctcctcgccggcctccgccagcgcttctgccagcaatgcagccggttccacgagctgtcgcacttcgacgacaccaagcgcagctgccgcctgcgcctggcggggcacaacgagcggcgccggaggaaccccgcggaggcgcaggaccagcacggcgggcggggcgacccggggagccaccacctccagatcagataa

SEQ ID NO: 96 - DNA, 1056 bp, Zea mays
  source 1..1056: mol_type = genomic DNA
atgtcattcatgtctccattcccggggatggatctaagtaaaatggatcccgggactctgaccctccttggggcagctggttgtgtaatgctgactatgcatttcactgtgcaattggtgtcacaacatctcttctattggaaaaaccccaaggagcagaaagccatacttattattgtgctgatggctccactatacgctatctcttccttcgtgggtcttcttgatatccagggtagcaagacatttttcacattcttagatgctgttaaagaatgctatgaggcactggtgattgctaagtttatggcattgatgtacagctacttgaatatatctatcagcaagaacatagtccctgatgaaatcaaagggagggtgcttcatcattccttccctgtttctcttttcctgcaaaagggtgttagggagggagaagagaggaagaacgggagggaagctggccagcaacctgcagccctccctgctggttctggagcctggtgcccgactcagtacttgctctggtcagctgcagcacggtcgatctggaaggggatgccccgcaatgtccgattggagcacaaaacattgaagcttctgaagtattggacctggcaatttgttattgttaggccagtatgttccattctgattattgcactccagcttcttgggctgtaccccagctgggtcagctggacgtttagtattatactgaacttttcagtctccatggccttatatgcattggttctcttctaccacttgtttgctaaagagctggcacctcacaagcctcttgcaaagttcttgtgcatcaaagggattgtcttcttctctttctggcagggttttgcgctcgatgttttaacccaagcaggcgtgatcaaatcccaccatttctggctggacgtggagcacatccaggaggcgatccagaacgtcctggtgatccttgagatggtggtgttctccgtcatccagcagtacgcataccacgtcgccccctacagcggcgctgacagggccaagtttgagaagaagaacgaatga

SEQ ID NO: 97 - DNA, 1482 bp, Zea mays
  source 1..1482: mol_type = genomic DNA
atggcgagcctcaacgtgctgctcctccctgagccgggctccggccacctcatgtcgctcatcgaagccgggaagcgcctgctgggccacggcgcgggcggcggggacggcctgggccgggagttcacggtgaccgtcctcgtcgtgcggccgccgacgccggagtcggccgccgaggtgggcgcgcacgtccggcgcgtcgcggagtccggcattgctggcgtccggttccaccacctcccggccgtggagcccccggcggactgcgccggcaacctgcaggagttcaagtcgcggtacctggagctccacaggccgcacgtggaggccgcggcgcgggagctcggcgccgcggcgctcgtggtcgacttcttcgccaccgccgtgctggacgtggcgcgcgggctcgccgtgccggcgtacgtctacttcacctccacggccgcgctgctcgcgctcacgctgaggctgccggcccttgccgagactgtcgccgccgacttcgagacgttcgagggcacggtcgacgtgccgggcctgccgcccgtgcccgcggcaagcgtcccggggttcctgggacgcaaggacagccccaacttcacgtggttcgtttaccacggccggcgtttcatggacgccgacggcatcgtcgtcaacacggtggccgagctcgaaccggggctgctcgccgccatcgccggaggccggtgcgtgccgggacggccggcgccgccgctgtacccgatcggtccggttctcaacctcggtgcggggggaggtgcgtccggcgacgaggcgtgcgtccggtggctcgacgcgcagccgcgtgcgtcggtggtgttcctctgcttcgggagcctgggctggttcgacgcggcgaaggcgcgcgaggccgcggcggggctggagcggagcgggcaccggttcctgtgggcgctgcgcgggcctcccgcggccggatcgcggcacccgtcggacgccgacctggacgagctcctccccgcggggttcctggagcggaccaggggcaggggcctcgtgtggccgcggtgggcgccgcagaaggcggtgctcgcccacccggccgtgggcggcttcgtgacccactgcgggtggaactcgacgatggagagcctgtggcacggcgtgccgctggcgccgtggccgctgtacgcggagcagcacctgaacgcgttcgagctcgtggccgtcgtgggcgtggccgtggccatggaggtggacaggaggcgcgacaacttcgtcgaggccgcggagctggagcgggcggtgagggccctcatgggcggcggcgaggaggggcgcaaggcgcgggagaaggcccacgagatgaaggccgcgtgccgcaaggccgttgaggagcgcgggtcgtcgtacgcggcgctggagaggctgcgtcatgcgattcgcaagggtgcaaatacagctactgctcatttcaaggcgccgcagccgctcatttga

SEQ ID NO: 98 - DNA, 1443 bp, Zea mays
  source 1..1443: mol_type = genomic DNA
atggcgacgcccgcgctggtgctcctaccggagtgggggtccggccacctcatgtccatgctcgagtcctgtaagcgcgtcctcctcagcagcagcggcggcaaggccttctccatcaccctgctcgtgatgcgcccgccgacggccgaggccacctccgaggtggaggcgcacgtgcggcgcgaggccgcgtgcgggctggacatccgcttccaccgcctccccgccgtggaggcccccaccgacgccgtcggggtcgaggagttcatcgcgcgctacatcggcctccacgcgccgcacgtccgtgacgccgtggccgccatggcgtgccccgtggccgcgctcgtgctcgacctcttcgccgcgccgctggtggacgtggcgcggggcctcggggtgccgtcctacgtcttcatgtcgtccacgggggccatgctcgcgctcatgctgcacctgcccgtgctccacgagtccgtccccgccgtggacttcgacgagatggagggggacgtgccgctgccggggctgccgccggtgccgccggactccatgccgtgccccgtggcggacaagaggagccccaactacgcgtgcttcgtgcgcctcggcgaccgcttcatggacgccacgggggtcatcgccaacacagcggacgagctcgagccggggcccctggcggccgtcgcccggggccggtgcgtgcccggccggcccgcgccgccggtgtacccgatcggccccgtgctgtcgctcggcggccgctcgccgtctcacgagtgcgtcgcgtggctggacgcgcagccgccggggtccgttgcgttcctctgtttcgggagcatggggtggttcgacccgccgcaggttgcggagatcactgccgcgctcgagcggtgcggccaccgcttcctgtgggtcctgcggggaccaccctcggacgcgtccgggtcggagcacccgacggacgcgaacctcgacgagctgctcccggaggggttcttggagaggacggggggcaagggcctggtgtggccgacgtgggcgccgcagaaggagatcctggcgcaccccgccgtgggcggcttcgtgacgcacggcggctggaactcggtgctggagagcctgtggcacggcgtgcccatggcgccgtggccgctgtacgcggagcagcacctgaacgcgtttcagctggtggccgacatgggcgtggccgtgccgctcaaggtggacaggaaacggggcaacttcgtggtggccgcggagctcgagcgcgctgtgcggtccctgatggacgccgacggcgagcaggggaggagggcgagggagaaggccgcgcgtatgaaggccgtgtgccggaaggccgtggaggagggcgggtcgtcgcacgcggcgctgcagagactcgcagaggcgctccacgacggcgccgtgatcccgaagaagtga

SEQ ID NO: 99 - DNA, 101 bp, synthetic construct
  source 1..101: mol_type = other DNA
gcttaattgatctatgtctaaattactattgttagcatataattcaattcyaaggatccaaactgggcctaatgatatttcaaacaatcctcaaaatacat

SEQ ID NO: 100 - DNA, 20 bp, synthetic construct
  source 1..20: mol_type = other DNA
aattcaattctaaggatcca

SEQ ID NO: 101 - DNA, 17 bp, synthetic construct
  source 1..17: mol_type = other DNA
tcaattccaaggatcca

SEQ ID NO: 102 - DNA, 16 bp, synthetic construct
  source 1..16: mol_type = other DNA
gctcgctccctgacca

SEQ ID NO: 103 - DNA, 20 bp, synthetic construct
  source 1..20: mol_type = other DNA
atcattaggcccagtttgga

SEQ ID NO: 104 - DNA, 101 bp, synthetic construct
  source 1..101: mol_type = other DNA
tactcacatggttggcacagagattcaaatgcctcccacctcccacgttgytgcacagtgttttgcccatcagaatatttcatgacacttcctaagacaga

SEQ ID NO: 105 - DNA, 14 bp, synthetic construct
  source 1..14: mol_type = other DNA
cccacgttgttgca

SEQ ID NO: 106 - DNA, 14 bp, synthetic construct
  source 1..14: mol_type = other DNA
cgttgctgcacagt

SEQ ID NO: 107 - DNA, 20 bp, synthetic construct
  source 1..20: mol_type = other DNA
tggcacagagattcaaatgc

SEQ ID NO: 108 - DNA, 20 bp, synthetic construct
  source 1..20: mol_type = other DNA
cgcgtgatctgtcttaggaa

SEQ ID NO: 109 - DNA, 1233 bp, Zea mays
  source 1..1233: mol_type = other DNA
atggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 110 - protein, 410 aa, Zea mays
MAIILVRAASPGLSAADSISHQGTLQCSTLLKTKRPAARRWMPCSLLGLHPWEAGRPSPAVYSSLAVNPAGEAVVSSEQKVYDVVLKQAALLKRQLRTPVLDARPQDMDMPRNGLKEAYDRCGEICEEYAKTFYLGTMLMTEERRRAIWAIYVWCRRTDELVDGPNANYITPTALDRWEKRLEDLFTGRPYDMLDAALSDTISRFPIDIQPFRDMIEGMRSDLRKTRYNNFDELYMYCYYVAGTVGLMSVPVMGIATESKATTESVYSAALALGIANQLTNILRDVGEDARRGRIYLPQDELAQAGLSDEDIFKGVVTNRWRNFMKRQIKRARMFFEEAERGVTELSQASRWPVWASLLLYRQILDEIEANDYNNFTKRAYVGKGKKLLALPVAYGKSLLLPCSLRNGQT

SEQ ID NO: 111 - DNA, 1653 bp, Zea mays
  source 1..1653: mol_type = other DNA
atggggacggaggcggagcagccggacatggacagccaccgaaacgacggcgtcgtggtggtgccagcgccgcgcccgcgtaaggggctcgcttcctgggcgctcgacctgcttgagtccctcgccgtgcgcctcggccacgacaagaccaagccgctccactggctctccggcaacttcgcccccgtcgtcgaggagaccccgccggccccaaaccttaccgtccgcggacacctcccggagtgcttgaatggagagtttgtcagggttgggcctaatccgaagtttgctcctgttgcggggtatcactggtttgatggagacgggatgattcatgccatgcgtattaaggatggaaaagctacctatgtatcaagatatgtgaagactgcccgcctcaaacaagaggagtattttggtggagcaaagtttatgaagattggagaccttaagggattttttggattgtttatggtccaaatgcagcaacttcggaaaaaattcaaagtcttggattttacctatggatttgggacagctaatactgcacttatatatcatcatggtaaactcatggccttgtcagaagcagataagccatatgttgttaaggtccttgaagatggagacttgcagactcttggcttgttggattatgacaaaaggttgaaacattcttttactgcccatccaaaggttgacccttttacagatgaaatgttcacattcggatattcacatgaacctccatactgtacataccgtgtgattaacaaagaaggagctatgcttgatcctgtgccaataacaataccggaatctgtaatgatgcatgattttgccatcacagagaattactctatttttatggacctccctttattgttccgaccaaaggaaatggtgaagaacggtgagtttatctacaagtttgatcctacaaagaaaggtcgttttggtattctcccccgctatgcaaaggatgacaaactcatcagatggtttcaactccctaattgtttcatattccataatgctaatgcttgggaagagggtgatgaagttgttctaattacctgccgccttgagaatccagatttggacaaggtgaatggatatcaaagtgacaagctcgaaaacttcgggaatgagctgtacgagatgagattcaacatgaaaacgggtgctgcttcacaaaagcaattgtctgtttctgctgtggattttcctcgtgttaatgagagctatactggcagaaagcagcggtatgtctactgcactatacttgacagcattgcgaaggtgactggcatcataaagtttgatctgcatgctgaaccggaaagtggtgtgaaagtacttgaagtgggaggaaatgtacaaggcatatatgacctgggacctggtagatttggttcagaggcgatttttgttcccaagcatccaggtgtgtctggagaagaagatgacggctatttgatattctttgtacacgacgagaacacagggaaatctgaagtaaatgttatcgatgcaaagacaatgtctgctgatccagttgcggtggttgagcttcctaatagggttccttatggattccatgccttttttgtaactgaggaccaactggctcgacaggcggaggggcagtga

SEQ ID NO: 112 - protein, 550 aa, Zea mays
MGTEAEQPDMDSHRNDGVVVVPAPRPRKGLASWALDLLESLAVRLGHDKTKPLHWLSGNFAPVVEETPPAPNLTVRGHLPECLNGEFVRVGPNPKFAPVAGYHWFDGDGMIHAMRIKDGKATYVSRYVKTARLKQEEYFGGAKFMKIGDLKGFFGLFMVQMQQLRKKFKVLDFTYGFGTANTALIYHHGKLMALSEADKPYVVKVLEDGDLQTLGLLDYDKRLKHSFTAHPKVDPFTDEMFTFGYSHEPPYCTYRVINKEGAMLDPVPITIPESVMMHDFAITENYSIFMDLPLLFRPKEMVKNGEFIYKFDPTKKGRFGILPRYAKDDKLIRWFQLPNCFIFHNANAWEEGDEVVLITCRLENPDLDKVNGYQSDKLENFGNELYEMRFNMKTGAASQKQLSVSAVDFPRVNESYTGRKQRYVYCTILDSIAKVTGIIKFDLHAEPESGVKVLEVGGNVQGIYDLGPGRFGSEAIFVPKHPGVSGEEDDGYLIFFVHDENTGKSEVNVIDAKTMSADPVAVVELPNRVPYGFHAFFVTEDQLARQAEGQ

SEQ ID NO: 113 - DNA, 876 bp, Zea mays
  source 1..876: mol_type = other DNA
atggggagggcgccgtgctgcgagaaggtggggctcaagcgagggaggtggacggcggaagaggaccagttacttgccaactacattgcggagcacggcgaggggtcctggaggtcgctgcccaagaatgcaggcctgctccggtgcggcaagagctgccggctccggtggatcaactaccttcgggcggacgtcaagagggggaacatctccaaggaggaagaagacatcatcatcaagctccacgccaccctcggcaacaggtggtccctgatcgccagccacctccccggccgaacagacaacgagatcaagaactactggaactcgcacctcagccggcagatccacacgtaccgccggaaatacaccgccgggcctgacgacaccgccatcgccatcgacatgagcaagctgcagagcgccgacaggcggcgcggcggcaggaccccgggccggccgccgaaggctagcgccagcaggaccaagcaggcggacgccgatcagcccggcggcgaggcgaaaggcccggccgcggcggcgtcgagcccgcggcacagcgacgtggtgaacccgggcccgaaccagcccaacagcagcagcggcagcacgggcacggccgaggaggaggggcccagcagcgaggacgcgagcgggccgtgggtgctggagccgatagagctcggggacctagtctggggggaggccgacagcgagatggacgccctgatgcctatcgggcccggcggccacgactcggctgccctcgaagggcttggcgcggtcggctgcgaggcccaggtggacgacctgttcgacatggactgggatggcttcgcggcccatctgtggggcgggccggagcaggacgagcac

SEQ ID NO: 114 - protein, 292 aa, Zea mays
MGRAPCCEKVGLKRGRWTAEEDQLLANYIAEHGEGSWRSLPKNAGLLRCGKSCRLRWINYLRADVKRGNISKEEEDIIIKLHATLGNRWSLIASHLPGRTDNEIKNYWNSHLSRQIHTYRRKYTAGPDDTAIAIDMSKLQSADRRRGGRTPGRPPKASASRTKQADADQPGGEAKGPAAAASSPRHSDVVNPGPNQPNSSSGSTGTAEEEGPSSEDASGPWVLEPIELGDLVWGEADSEMDALMPIGPGGHDSAALEGLGAVGCEAQVDDLFDMDWDGFAAHLWGGPEQDEH

SEQ ID NO: 115 - DNA, 401 bp, Zea mays
  source 1..401: mol_type = genomic DNA
attttgttaattgaaaatgatattggtagaacacatgtaaattgtatcagtagggataaggcataacacaagaaaactatacatgaaaatatgagtagttgttaatcacaaggtaccttcgaatttgyggttgaaggtatagttctccaagatcacgtgggacttctttatgccagaacctatatataataaaaaaacagyaattgttaataaakgytgtagctatatgagttgggtagcagggcagcaaccaaacacatgccccaasaacatactattcatattttagtatgtgtgaactgcaagcatattgaaactacactcaaccaaacacacgccccaacactcaactattcatgtgacactacaagcactaaaactacaccatgtatttactatga

SEQ ID NO: 116 - DNA, 401 bp, Zea mays
  source 1..401: mol_type = genomic DNA
gagcgtggtcggaccngcgcactcccagtcggagccgtcgccgcgctccgcctctcgcgcattcaccggccgtagccgcaccgatactaggatccnctcctcgctagccattgctgtactccrcgtctgcttttcttgctatggatccaaccttaaaggaacacaaacaaagcgactgacccatcaatggatgcycagtcrcycacatgcgtcmcgcacawtggcaygaccgaaccagcgtcatgttyatgcgtatcgattgcaaaatgcagcgagagccggcaaaagcgnccaacactggtaatgtaggcaggccgacaagcagtgacccgtgaagagcgtggtcggtgctgaccccgcggatggcctccctgcgcggaagttgtggctcggtacgatac

SEQ ID NO: 117 - DNA, 401 bp, Zea mays
  source 1..401: mol_type = genomic DNA
cacacgtgccaaatatgttttgggcagggactgtgtgattttgccaagttgtattacgaattgtctgaggtttgagcttggacaataytgtttctcaggtcgtggaccgtccgatcttgttgggcggaccgttcgcctgtgtaatagttatgtgggcagagctcgggttttgaaagttgccatgtcttggaccttttagtyagggtcagcggaccatcggaacctgacttttctgacagctctgacatgtttcaaacggaaattatagcagttagttgtacgatggaccgtccgtccagaaggcgcagaccttctgcaagtgcgcaaaagggcatatcgcccataacgactaatattgaatggggtagtataaatagaagtggtgctcgtgtatggaaatt

SEQ ID NO: 118 - DNA, 401 bp, Zea mays
  source 1..401: mol_type = genomic DNA
agagaaagctccaactgatgtgctgggatggagcccttctcatcatttcccctagatgttgttctcatagtatccacctcccactgtgtgttatacttctgctgattttccataatgtggactagctccatatctcttgaagctctctcagaattattctcattcaaacacgttcttttctttggcgataaatcgtcttckcttgctggratcaagttattaccctyagntcctgncacatcacaagcacgttacgttagtaaatttcctggggccaaattgtacatcacaagttgaaaagatggggaaaattactttgtattttggtctgtccatttagctcaacattcttgtggctaggaatagacaagtgcctttgagttgattgcttatantccaaa

SEQ ID NO: 119 - DNA, 401 bp, Zea mays
  source 1..401: mol_type = genomic DNA
ataaccgaggtgaagtttatgcacggctgcgtacgtatatacacaaatataatggtctagcctatagaccgtagagagaggtccatctcattgtcatgcatgacttcataggtctcctctctcccatctcttcggctcttttttacgcacccagtaaggccttgttcgtttatgtcggattgtacccggaatcgtttcagmtagtcaaagtttatataaattagagaagtgaattgttccgacccaccaattcggcacaaacgaacaaggccnnaaagtctatcgatctcatctccgaggctttttacatttcacgtcgtcagagcaaatctttttcgcttttcgttccttgccaattcagtttcttcccttttcgtacgttcacccacctttcttgtttt

SEQ ID NO: 120 - DNA, 401 bp, Zea mays
  source 1..401: mol_type = genomic DNA
gagcgtgatctaagtgggcatcsgccttcgaaagcatggtcgagtacttcctcgatgcgcttcacaagcagaatctgacagcagaaaaatatatttgataagcacygaattaaaangccggtttcaccaactncttccaaaattacatatyaagaggttttatgtttgaatctagtttgtggatgcccctaaactgcaggwaaatctagctttagttttagtagctccaaggytcagaaancgtcgaagatcaaacaacaccttagtaccaaaaagctgcccaagnaacatgcaaacaacaatgcaacaacatacctccatgtcggagagaattngtaatggaacctctgataagtccttcaagttcctttcaggtaaaattactctcttgattccataac

SEQ ID NO: 121 - DNA, 401 bp, Zea mays
  source 1..401: mol_type = genomic DNA
cttcagcgacggcaaggcctcggcgtcaggccgacagagasaaaaggcattgcattggtctcgttaagcaagcagtcgggctagctayyaactcanccttctggacctggaagaactggagcgcgaccccgagcgcgtcgccgtacttggagtcgatggcccccggcggccccggcaccggcagcggcttgatgtggcggsccccgggcttgcgcttcatgatggcgaacatcgcggccgtggccaccagcgccgcaatcagcgccacgatcagccagccgcagcaccsgcncgtgtacttggaccccatctccgccggcgccgcgcctcctcctagagcattcgscgggaagccgcctcctcargtgttccgctgcgtttcggaggcctcscgstggccg

SEQ ID NO: 122 - DNA, 401 bp, Zea mays
  source 1..401: mol_type = genomic DNA
caatctattatcactgcctttacattcttgccttaggagtagcaggtagtcatcctcatcctcaatcttcacctcccctcaactctacgttgtttagaagcgtatgatccggacccaagacaaccctatgatctttcctcctgatgggatccctccccgagggcgagatccaggttctgtaagagctcatcgctttctccscacacatcgtggacggtccggtgacctaccgcggatggtccatcgcctcacagagaggttcctcaaggtggtctcgttcctcgtgaacgggatctagagatagtaagtgcttggcccgaaagggtgccaacacaagttctatgtagcattagcaactgaatatatgtaaaagttatagcagtacaaaaatgatgagaacc

SEQ ID NO: 123 - DNA, 401 bp, Zea mays
  source 1..401: mol_type = genomic DNA
gcgtgcccgnggtccgtcacgaatattagcaaccgctggcttggggctgcaggggcgggctggagctgctcctggagaactccaccaaggtgcacaaggtggaggtcaccacgcccaaggacggccaaggcaaggtgacgatgaagttcttgctctcttgggkcaaggagaatctgatcaaggagcgmccggagatgttcstcaaggctgattccgtgtacgtgttgttttgctccastttctcttcattcacttgtgtagntrgtaggrttgatatttaatgggcattgaaacaagacagattctgcgccaaggtcgtggtctgcttcagttgctgccgtttctcttatgatgaatagccttttcctgacggttaactagtgtggactgataattagttc

SEQ ID NO: 124 - DNA, 401 bp, Zea mays
  source 1..401: mol_type = genomic DNA
cccaaacagaacgtaggttacttcgatttggctgtcttaacctatctaagttttgtatcgtctcttatacaccatcataattcatcattcgagtcctttataaataaatccaccatgtggatcatcccrtgtgggctatacggtactaaacaccgacacaacaccaccaagctgtctactaagactggcccaaatacgttmctaaataaatttsacttgaaaatggttcttaacaatgtgcactctttagttactgtagtagaagtaaatggaatcaattcttaggccttgttcgtttgtgtcggattagtgggtcggaatgatttctaaccggattgtttctctaatttatattgttggcgtttcgagaccgggggggtctctgggccgacgagtgaaat

SEQ ID NO: 125 - DNA, 401 bp, Zea mays
  source 1..401: mol_type = genomic DNA
gcactgcgttgtcgtagatgtcgcttccagtyccagcatcyctccggtagatgctcagaaccttgctggcggctaccttgaaagccccttgctccatcagctctctggtgaggtgcttgtcccttttggggaggttcgccaggatctgaagcgcagcatcttgctcatntgctgcggcgtttccatcgtttacgaatgcarctttgactaggcagccaagcagcgtcggacttgcacggaagacttgggcgatctcgtastccatgtggaacgagattttgtgtatcagctttatggatgcggtgcggctttcctggtgatcgctctcgacaaactgcaccaggttggtgvtagcgccggaagattttatagccgagacgatcgatagcactgtccccgtg

SEQ ID NO: 126 - DNA, 401 bp, Zea mays
  source 1..401: mol_type = genomic DNA
agattccgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgatctagagattcgtcaacaaagaaatgttcagattttctcacttaacagtaagcctaaattymaaaaacgttcacacccaaattaaagarcctactattctaatacaggtcagcaaggcatgccacacaaagtcaaacaagactattgcccaagaatgccaagaagaaaaagaaacatgttgagtcaaagctattaabtcagtactaccaacttaaacctgcatgttgtgtgcaccaacataaaargcacaagggaaaaatcaggaataaatacagtacaaagaatcaca

SEQ ID NO: 127 - DNA, 401 bp, Zea mays
  source 1..401: mol_type = genomic DNA
aatctcaggtaccagatagctattggagttgcaagaggactgvcctacttgcacactagctgtcgggactgcatcatacaytgtgacatcaagccagagaacatactccttgacgcatcttttgtgcctaagatcgcagattttggaatggcaaaggttttggggagagagtttagccatgcggtgaccacaatgagaggsactattgggtacctcgcgcctgaatngattagtgggacagccgttacatctaaagtcgatgtgtacagctatgggatggttttgtttgaavtcatatcgggaaggaagaattcgagtccagagtactttggggatggcgattactcaagttttttccctatgcaggttgcacgcaagcttcgcagtggacasgttgagag

SEQ ID NO: 128 - DNA, 401 bp, Zea mays
  source 1..401: mol_type = genomic DNA
atgagaggcactattgggtacctcgcgcctgaatggattagtgggacagccgttacatctaaagtcgatgtgtacagctatgggatggttttgtttgaagtcatatcgggaaggaagaattcgagtccagagtactttggggatggcgattactcaagttttttccctatgcaggttgcacgcaagcttcgcagtggacasgttgagagtctggtggatgaaaagctgcagggcgatgtgaacctcaaggaggttgaaagagtatgcaaggctgcgtgttggtgcattcaggaaaacgagtctgctcgaccgancatggccgaggtggtgcagtttcttgagggcctctctgatctcggtatgccaccactgccaagactgctcaatgcggtcacaggagg

SEQ ID NO: 129 - DNA, 401 bp, Zea mays
  source 1..401: mol_type = genomic DNA
tgactgggtcyagaaaagttyggtgcttaagggagaaaattgaactgtgtttatctgccaagcactcatgctatgttgttattakttttttttamattgccttttcttactggtatatactattcaaaacytttcagattatacattggagggttcacaaagaagaatgtgaaaggtttgcagaacaaatgaggcacatyratgcgcngagccagtttccrttcacattcttggaayctcctgctctggtacttttctgtacccattgttttttttcatagctaagatcttctgggttgctattacctccctcgaaatgacatggtggaaggtactataaatttcagtgccttgcttcctaaactgcactttgcccacatggactgtctattttttctcta

SEQ ID NO: 130 - DNA, 401 bp, Zea mays
  source 1..401: mol_type = genomic DNA
ttcagttattgctagagagtgagaaaaccatgcaagcacctgcggttgaaaggatgagcagtgagatcactgttacaagagatcacgacgaagagcagcaggaggctctgcggcgtgcgatcatgctaggggttgctgatgtaaacatgccatcaacatatggtacgttcaacgaaaatcctacttctgaaccagccgagrcctccccattactatcagacagcggaaggcgcagggcagtatgggaggagtggatcgcaaggatcttcgagaaggatgaatcagggcaaatggttccacggagatgatcgtggcgacagctcaaggcatgcttgcttgatgggtcggtttcttgatgctgcttccactctgtttcgagcctattttgaaacttcggaacg

SEQ ID NO: 131 - DNA, 401 bp, Zea mays
  source 1..401: mol_type = genomic DNA
gtgggagcgagcatctgacatgaactcracgacctccttgcgaactggtggctccatgcgscccaggcgctgcgcgacaagctgcacagcctggtgkcgcagcatctcytgctgaactatygacagmggctgcgcgtaatgcatcttgggcaasaggggcctagagtatgtgaagagcacattgrtmagggacgcggctgwgaacctgatggcgagatggagctcgcccatcttcttgactcctgacgggscgaggacgagcaacggatacgagtgcgtgtacacacgccccgtctcaagygtcgaaagccggatcctsactttcccgatgggtttatccatgtggyskscwgaggarccgttgtctccacttatgtgacagttgtcaaacagaccaaygg

SEQ ID NO: 132 - DNA, 401 bp, Zea mays
  source 1..401: mol_type = genomic DNA
tgggcaasaggggcctagagtatgtgaagagcacattgrtmagggacgcggctgwgaacctgatggcgagatggagctcgcccatcttcttgactcctgacgggscgaggacgagcaacggatacgagtgcgtgtacacacgccccgtctcaagygtcgaaagccggatcctsactttcccgatgggtttatccatgtggyskscwgaggarccgttgtctccacttatgtgacagttgtcaaacagaccaayggtgagcacggttccgtgatcgaagacttcccacgtgtactgctcattgaaccgagggctcaggctgtcaactatcgtgcgcgtccgracccattttgaaccatacttggcaacacagtacgcrtcacaggacccacggtcgtttctg

SEQ ID NO: 133 - DNA, 401 bp, Zea mays
  source 1..401: mol_type = genomic DNA
gggcaasaggggcctagagtatgtgaagagcacattgrtmagggacgcggctgwgaacctgatggcgagatggagctcgcccatcttcttgactcctgacgggscgaggacgagcaacggatacgagtgcgtgtacacacgccccgtctcaagygtcgaaagccggatcctsactttcccgatgggtttatccatgtggyskscwgaggarccgttgtctccacttatgtgacagttgtcaaacagaccaayggtgagcacggttccgtgatcgaagacttcccacgtgtactgctcattgaaccgagggctcaggctgtcaactatcgtgcgcgtccgracccattttgaaccatacttggcaacacagtacgcrtcacaggacccacggtcgtttctgg

SEQ ID NO: 134 - DNA, 401 bp, Zea mays
  source 1..401: mol_type = genomic DNA
kscwgaggarccgttgtctccacttatgtgacagttgtcaaacagaccaayggtgagcacggttccgtgatcgaagacttcccacgtgtactgctcattgaaccgagggctcaggctgtcaactatcgtgcgcgtccgracccattttgaaccatacttggcaacacagtacgcrtcacaggacccacggtcgtttctggstttcgttgggttaaggccgttggcgcttaaaatgccaacctcaagcatgccgatgggtggcttccacagctgcttcatcgtcggacggagatcactgcagtactgtgtggactcgtcgagcacatggtagccgccttcgaggcagacrcggaggcttaccttcgcatagaacttgtcttccttcttcttgagcagctggt

SEQ ID NO: 135 - DNA, 401 bp, Zea mays
  source 1..401: mol_type = genomic DNA
gtycacatcgtcgatcagtcctggcctcctgagatcaacccactgcggcggcaccggcttgtggtcagcccrcctcggwagcctggcsagtggtatgacrgcttcaccgatcacctcgtctttgttctgtgccacacrgtcytstaccgacacgaccaggtcgtcctcgaaaggctctgccgcgacgaacatgagctcstynttccacatgaagtttctygtcggcgagcgaacctgcctcgtsytgagcagctgatggcccagcctcaccttgaccagmacatcangyacgnggttctccgccatcgggtatacgtcgtgcgcctctakcacgttgaccctsacgtaccacatcctcggcgcagggtagaccttcccgcggatgtggctgtggagatggg

SEQ ID NO: 136 - DNA, 401 bp, Zea mays
  source 1..401: mol_type = genomic DNA
tgccacacrgtcytstaccgacacgaccaggtcgtcctcgaaaggctctgccgcgacgaacatgagctcstynttccacatgaagtttctygtcggcgagcgaacctgcctcgtsytgagcagctgatggcccagcctcaccttgaccagmacatcangyacgnggttctccgccatcgggtatacgtcgtgcgcctctakcacgttgaccctsacgtaccacatcctcggcgcagggtagaccttcccgcggatgtggctgtggagatgggactccaccggcgttgaccccgcatggatggcgctcgggaagcactcgtctgcctgggtgccrtaccasaccgcgagcatcagctcccctctgtccctcaygccgtccttgcccacgagacggtaccact

SEQ ID NO: 137 - DNA, 401 bp, Zea mays
  source 1..401: mol_type = genomic DNA
cgccatcgggtatacgtcgtgcgcctctakcacgttgaccctsacgtaccacatcctcggcgcagggtagaccttcccgcggatgtggctgtggagatgggactccaccggcgttgaccccgcatggatggcgctcgggaagcactcgtctgcctgggtgccrtaccasaccgcgagcatcagctcccctctgtccctcaygccgtccttgcccacgagacggtaccactccggygccagcggactgtcygggggcaccctcagggggacctckctcaggtcaacrctcactcggccgacgtagtcgtcccggatgacgtccttrtccttgacgacgacctcgagcgtcgycgactgcatgacctccttggggaacgcgaagacctcgtcccactcgggcc

SEQ ID NO: 138 - DNA, 401 bp, Zea mays
  source 1..401: mol_type = genomic DNA
gatgcatctgtattctcgctcctcttccgccggcatgcgaagcatycgccgggagctccagcgcaggaggtccaagcccttggcgccgacagggtcggtcgctaavatgycgtccgcgtcgygtcacgcttcgagcggcggcggcgtccgggatccatgtcctcgtccacygcatcgagcggccccgtcttcgacaatccrccatttccaaggcaccacagtcacygacgcgtcacaacaccgtcggcctcgcgcttcgtcgcgctgcacccccgcctctcctccgccacctcggyctgccccatctccwrgcgtccccgcgcatctgaagcctgggacggtggttggtgtccgcacgaggactacaacgctgaagacggggcaggttctcgtgctgtggc

SEQ ID NO: 139 - DNA, 401 bp, Zea mays
  source 1..401: mol_type = genomic DNA
cttgcaaaaatgccagtatctttttctaagtgaaagaagatgttgtcaccggagcatgctcttggattgatgcgctgaccttttcgcgccaatttttcagaaacccaaracgtggagattggcgtgccaggcaacsgtgggcaacgcagattcgactggacaggtatgcacaatcgtctttcacgtagcgtattccccagrcgtggtagaaaatcaaactgtccgtacgtcctgaaagttgctctgaaaaaacactctgagatttggtatctgcgcagatgatcattcaacagttgccggagtggaagatacacgagtgggacaagtaggggttactgctttactggtcttgaagagtgctctcgtggccatactatagattacatgcantggaaatggga

SEQ ID NO: 140 - DNA, 401 bp, Zea mays
  source 1..401: mol_type = genomic DNA
ctttgtgtcctcagtacaataagcccattctgaggatatctcagtgatctgaaagggtatttygccagtcacctgttcagtggggagtatgcccgagcttggattatcatctaaactratatcatcatccaagtatttccataacccaagactgttctgcctagcaggtacatcatttggcatgttatcagacaccggagytttcatcaaagaatttgtatgttgctttgattgtnaatccagatctgatgttaaaagagatggagcatggtttgcttgatctttcagagatccaaagtaactctctgatacttgactaaggccaggattagcataagcttctgaaaccacattagttggtggagaaaagatagcctgccaaaaaaagatgttgattcatc

SEQ ID NO: 141 - DNA, 401 bp, Zea mays
  source 1..401: mol_type = genomic DNA
acattgcaatttgacttgagacaaagctaagtgcactataatttgaaacggatggagaatgagatagatgtgttgctgyctttctttcacaatagaagtcttctgaagttatgtgtgatgggtacattkattgaatgctagaaatatttatgctgaggaaggttcagargctatggatgttgctgctgcccctgctgtgcrggtaagcttcgtttcctgatgaggacaaytacactgtsaaaacatagccatttttcagctttcacaatctaaagctacttattctgttaatttttaggcattggcaagatacacacgacgagaggcaacttgtctgtcattgactggttaggtttttgcaactctaaacacatttrtaattttcacctcgaataggttgc

SEQ ID NO: 142 - DNA, 401 bp, Zea mays
  source 1..401: mol_type = genomic DNA
gcattggcaagatacacacgacgagaggcaacttgtctgtcattgactggttaggtttttgcaactctaaacacatttrtaattttcacctcgaataggttgcactcactggatacttcttgtctggtatattctaactacagacagagattctatgttctctggaaacttcaaattcacaaccctggtgccggcaactcycagccccagcccaaagaagcatgggcgatgaactgccgtggtacatcaatatttcatacgaagattttwaaacagcaggatcagtggatatatgtcattctctcagcaatacaaataacaggcttcttccattgattgtttattccggggtcctcacgattcgaacccttttgatggaaatacttcgttctagataggat

SEQ ID NO: 143 - DNA, 401 bp, Zea mays
  source 1..401: mol_type = genomic DNA
tttaaagacarttaaatatcawaccatgrccatgagttcatcaaaataattgttatmcatcagggtcaaatcactaatsaatgsctgccattaaactacaatgaccctgatgaaacacaatacatgcaatcaaaagtataaynatgtgaaaaggacattatcacttacctccacggcgctttcgtgaagcaatgttgtcaktgttttcagctaatgctccacsagaccatggrctagaaacctattagacaaaacatgatcacaatcccccaataaaaaagacaatacaagtttatggtttygctgaagacaaaccggcattggagaacctctaaacccatagctatatcggcctagctcaggtcctgggccatatcccatacctacaacaattacaataa

SEQ ID NO: 144 - DNA, 401 bp, Zea mays
  source 1..401: mol_type = genomic DNA
cattggcccaggtggaccatatgaagagaaaagaccrtatgaaccaggaggtcctactggtggaccatagccatatcgcatcccaaggtgtggatatggagcaccatagctaccagttccaagcggaattggaggtggggcgcyaccacctcctccgtaaaatagaggtgaccrgtcatatccagaagcaggaggtcctgrcatcattctctgcaaaacaaacataagttaataaaaggacatctcaaycagttgaatagttgataacccakacatgatgcataaacatatccctttcacgtggctaccatgaaacacatcattttttaagcaatcagtgtcataagtattcatcataactctttgactagcagatggtagcgggtgctcgtataacatat

SEQ ID NO: 145 - DNA, 401 bp, Zea mays
  source 1..401: mol_type = genomic DNA
tctacattctcwaactaggacaaattgcatcatgcacttgyagttgtgctcactgaagttaatgcagtttcatttcttttatttactgtgctrctgaaccaaccaaaaactcaaaacttactaaattaaactaaaagagtgtgatctgatctggttgacgtawttcatttctcgatgctgaattgatcacttgcagcatnygctggctctgcaggctcacgatgtcttcaaggcgaaagaggtggtcctgcaaaagaagatctctcaagaggtggaacgggccaaggagttcacaaagtcgggaaacaagcaggcagcaatgcagtgcctgaagaggaagaggtactacgagagtcaaatgaaccaagttggaagcnttcagctgcgcattaacaccaagg

SEQ ID NO: 146 - DNA, 401 bp, Zea mays
  source 1..401: mol_type = genomic DNA
cgagaagcctcattcctyctactcaagatcaccagcagaggacaggaagagatccttccatcacgtccacttcacaagaattgtggggttctctatcgtcatacatgaggtcaaggcaacttggtccagtactagcaatgtcactggtagctctcattatcctaatgcaggcaagtattctggagactagatttggtctwyctgctggttcttgstcacctatttgtgcgcccagtagctacakctgaacataagcgatggcatgaaatgatgaaaatgttcagtgaccgccaacggttctgacacatmtctctgttgcaggtgaccatcatagttctactaacaagacccccccaggtccaaatggatcctcatgggatctggacgggcagttcagggta

SEQ ID NO: 147 - DNA, 401 bp, Zea mays
  source 1..401: mol_type = genomic DNA
ccatgctgcacacagatgatacaagaacaaacttggtctggaaagcggaaaggtggagnrtttggggccacaggttcctgacccyggagaagagagcgtgcacaaggcacgtccactggtgtgatgtgcgggaagtgaacgggagcgacggcgaacatcacccaaggttgcatgctgctgagtgacttgctgaaccctctsctttattgagatgaccagcttattgcccttgcacatccatcacgacttgcatccaaacgaagtatacaccaactacrcacggcttgctatctgaattatgtccacaattttctttcaayccagagctacyagctgcctcatatatgtgaaacgaagacgagcaacatcgcktgtcaaaactgttgaaagcaaatggaatt

SEQ ID NO: 148 - DNA, 401 bp, Zea mays
  source 1..401: mol_type = genomic DNA
gttacctctgaaagcactcagacartcctctagatttattgtcaataacagctgcgtatttggcattggcttcatcaaagttgccactatcaaagtyaacacgacgctagaaacactgtttgtcaggaagatgaacatcttcaacgaggccaaagtttacacttggaaaatcgaggatttctctgctctgaagaacccgtsccactcacsrgagtttgagatcgctggatacacatggtrataaatratcttttcagcttgcagtacttgacatgtactccacatgactgaacgttgataycataatctgaacttccttgcaatgctaakagtggttctgaaccacatcgkttttgaaaaytttacttgaaacacaattrtagcagtcctgcacgtcgtca

SEQ ID NO: 149 - DNA, 401 bp, Zea mays
  source 1..401: mol_type = genomic DNA
aatcacagccacatcatcttttatatttcataattgtgggtaaatcagaagccgacacctcactgtcgccccttgctctcgcccgcgaagactataaggaaaatggaccccatgtcatttggctcaataggtttttgtgtttgatgatcaacacaacctgtgtactaatgtgtttgctagtgtttatatttgtagttcacrggatgctaaggttacttagactaaggcaatgaggaaagcaacaactcaaaagacattatgaagatcacaagtgaagatcaacaggtatcaaacaaagtccaaggaacgaagaatagtgttgccaccggcggactgtccggctaagaagcaccggactgtccagtgcaccagggaacaggagcccaacggctagttccagg

SEQ ID NO: 150 - DNA, 401 bp, Zea mays
  source 1..401: mol_type = genomic DNA
aaaggtaagaagccgcctcaaaaccagggcttcgcaaactccacrtctaaatcwgggagtgccggctctttttctcttggcctttcctaawttctaagctcaaaaattcaaccgcgccagaacaacatcatcaataggagattgacagattgggcagctcatgccgctcgatttcagctgatcggcgcagtcaaaacaggygcacacatgcccacacctgatccatcacaaggaatgcggttagaagcaagtccacacaccaatttgtgcacttcwtacgattgttagttagrtctggaggttmtttgtggacaaagatgttactaatcataacgagaataagatatgacttcttgattaaagttactccargtactagtagatgatctatccgactatcc

SEQ ID NO: 151 - DNA, 401 bp, Zea mays
  source 1..401: mol_type = genomic DNA
aactggtacttttaagatgaaatwccgtactagcaaaagtagaatgkcccgtttgcatcccagattcccaatgctttcatcaaygtagggataaygtgtgattaaatgcatgtgactttgcaaaagcaatatgcaaaacagttagactgggaccaatttacctctcaagaaggttgcaaatgtcagcattgtgtgacagascatgayagtctgattgcgtgacaacacatagtgatctccacgagttaggggactctatccaggggcggtcattgaattgaatyggattattatgattctcaatatcacccattgtttcctgatgccagctatccggactcatagtatcaattggtgcaggccaacctaggctgcttatccataaccctctctcttcccag

SEQ ID NO: 152 - DNA, 401 bp, Zea mays
  source 1..401: mol_type = genomic DNA
acgswtccgcacgtcgcgcgcccgatcgtgtgagctcctgcatrtatagtgcatgcatgtcgycactgttagcattaacaagcatgtgtgtgcgtgtgtgtgtccgcgcgcgaacgatcgatcgatcgatatatgcattgctctcgcactcacgactcaccggaaagcgcgacsaggtcttggatgttgagacccttygtmtcgaagaaggcgacgagctcggtgacgcgctggccgcccatggggacgtsgcggtcggccgcctcggmgctggagtagtccttcctgtccctgcgcccgtacttgagcgaccagtagggcgccccggccgagaaggcggcgtcgcgrgcggckgcggtsaggatgtcggcgcaggagacggtggcgcggcacttggcctc

SEQ ID NO: 153 - DNA, 401 bp, Zea mays
  source 1..401: mol_type = genomic DNA
gctcggtgacgcgctggccgcccatggggacgtsgcggtcggccgcctcggmgctggagtagtccttcctgtccctgcgcccgtacttgagcgaccagtagggcgccccggccgagaaggcggcgtcgcgrgcggckgcggtsaggatgtcggcgcaggagacggtggcgcggcacttggcctccacctcyttcttgatcycctcgatcagctcgaacccgcgsagcgtcctgctcgcctccgcgtacttctcgctctgcccgggcacgtctaccaggaccgacccgtccacgccctgcannacacaccaccatgtcarttatattygccgaccaccaacgaaagatacacacgtacgtacgtaccccgacggcgaagtcgtngaagaagaggcggatgat

SEQ ID NO: 154 - DNA, 401 bp, Zea mays
  source 1..401: mol_type = genomic DNA
gtcarttatattygccgaccaccaacgaaagatacacacgtacgtacgtaccccgacggcgaagtcgtngaagaagaggcggatgatgctkgcggcgagggtctcgtcggctttcacggccttcatcacggccgtcccgacgatctcctccattttcggncacgacytctcgtagtaccgctcgtcgaggccgtccacggrcttctggtacggyggcaccgtgtagcttccctcmtggccgcygccgttcgaaccggctgcagcgccgtarccgttggaactctyccccggngaagcggcgtagccggtcgcggccaccgagacggcgacgasgagraggaacccgkagagggcaagrmacgacgacgakacascccgcggcgtcctcatcttgctctgtc

SEQ ID NO: 155 - DNA, 401 bp, Zea mays
  source 1..401: mol_type = genomic DNA
agtaccgctcgtcgaggccgtccacggrcttctggtacggyggcaccgtgtagcttccctcmtggccgcygccgttcgaaccggctgcagcgccgtarccgttggaactctyccccggngaagcggcgtagccggtcgcggccaccgagacggcgacgasgagraggaacccgkagagggcaagrmacgacgacgakacascccgcggcgtcctcatcttgctctgtcgatcgccrgccgagggtagtcctcaggytycttctacctcgtgatggaggatggcactgatctgggcttccaktgcgccaggatactccttgtttatatgtacaatgtactactggtgcaccgcggttacctccccgtacatgcagacacgtactcctagcttacaaagacgt

SEQ ID NO: 156 - DNA, 401 bp, Zea mays
  source 1..401: mol_type = genomic DNA
tgatattaaaacatacagggtcaaacaagtgggggaaaatgctcctggcctttatatgaaacaagctrcaacaaaagccytamaacmagcyatcagcaaaattctattctaccagtagattcatgcttcatactcatatyccccctkaaaaaaaagttagggatawtttctccggtgagctcttgagctmtctcaactagytctcaagtgmctcsagactcaawnctagcaaatcgttccatgtatccgttggtcctggcaaacaccaatgcatgcaatcatcgtgttttttcccgccagttgttgaagggtgggcatcagctctgaattcactcatgcgagtgatgtccagaactctgaaactagwctgttgaagagccttcaycaagtgttggtttacc

SEQ ID NO: 157 - DNA, 401 bp, Zea mays
  source 1..401: mol_type = genomic DNA
ttctcattctttatgttgcaattkccmtaccrgataacacaggaacatgacaakgaagtatttcagcacattcccatatccatgcatcatattatgcatgatgttgkaacaaaaawtgaagaaayakmwtgcatgaggatatrmaaagtttgctattttactaagcatatcatgtacttagtatcgtatcagtacctcttytgacgacaagggctgattacrttggcaagatccaccttcattccagtcaccaccttcraaatgtctaggagattgagtgcggaaraatttcactgygtttggtcttrttgctttgktsrcgaaggatatctgatggaaawttttcattcagagcatgrgaaagataggccgratrycattcacatagactgrcataggta

SEQ ID NO: 158 - DNA, 401 bp, Zea mays
  source 1..401: mol_type = genomic DNA
ttkccmtaccrgataacacaggaacatgacaakgaagtatttcagcacattcccatatccatgcatcatattatgcatgatgttgkaacaaaaawtgaagaaayakmwtgcatgaggatatrmaaagtttgctattttactaagcatatcatgtacttagtatcgtatcagtacctcttytgacgacaagggctgattacrttggcaagatccaccttcattccagtcaccaccttcraaatgtctaggagattgagtgcggaaraatttcactgygtttggtcttrttgctttgktsrcgaaggatatctgatggaaawttttcattcagagcatgrgaaagataggccgratrycattcacatagactgrcataggtagcwttttttaagagatasaaa

SEQ ID NO: 159 - DNA, 401 bp, Zea mays
  source 1..401: mol_type = genomic DNA
catgatgttgkaacaaaaawtgaagaaayakmwtgcatgaggatatrmaaagtttgctattttactaagcatatcatgtacttagtatcgtatcagtacctcttytgacgacaagggctgattacrttggcaagatccaccttcattccagtcaccaccttcraaatgtctaggagattgagtgcggaaraatttcactgygtttggtcttrttgctttgktsrcgaaggatatctgatggaaawttttcattcagagcatgrgaaagataggccgratrycattcacatagactgrcataggtagcwttttttaagagatasaaaygttataattccatttagatcaaattcacatggtcaaagaaaaawttcccaatyagttgtakaatatccatgycc

SEQ ID NO: 160 - DNA, 401 bp, Zea mays
  source 1..401: mol_type = genomic DNA
cacaccagcaagcaaaggcattctacatacccgggaaccctaacacccgaatcggttctttgaaarctactcnaccacaccacagcacattgtccccaccaagccgcatcgcaygtcaaatttctgaggtgtttttcaaagacagcgaaacggggggtgacaagttaccgatactggtgtccctgtggcgctccaggaacsggcgcgggtcgaggcgggggagctcgcacccrggccccgcgggcgtccaccgccagctgascaagtcgcgggcgttacccttgccattggcgatgcagttccakcccttgaagatctccttgcaggtatggtcgtagcggagcgaggagcccgcgaaggggtcgcgcacccactcgccggcggagtagtcgcaggcctga

SEQ ID NO: 161 - DNA, 401 bp, Zea mays
  source 1..401: mol_type = genomic DNA
gccaccaacaactccttcgacggcgcgatcccagcgcagttmggccggtcgtcggggctccagcgcgtgcgcctggggagcaacatgctctccggaccgatcccgccgtcgctcggcggcatcrccgcgctracgctgctggacgtgtcaagcaacgcgctyacyggcggcttcccancgacgctcgcgcagtgcmcgmakctcagcctcgtcgtcctcagccacaaccgcctgtcaggggcgattccggactggctgggctcgctgccgcagctcggcgagctgacactctccaacaacgagttcaccggagcaatcccggtgcagctcagyaactgctccaatctcctgaagctgtcgctcgacaacaaccagatcaatggaacagtgccgcctgaact

SEQ ID NO: 162 - DNA, 401 bp, Zea mays
  source 1..401: mol_type = genomic DNA
ccaagctggaagacctgaacctgtcccacaaygctctggtcggcgcggtgccgtcgcagctcgctggaatgagtagcttggtgcagctggacctgtccagcaaccagcttgaagggaggytgggcattgagttcggccggtggccccaggccgcgttygccaacaatgcagggctctgcggtagccctctragaggttgcrgcagcaggaacagccggtcsgnrttccacgcggcgtccgtcgcgttggtgactgcggtggtcacgctgttgatcgtgctcgtgatcatcgtgcttgcgctgatggcggtgcgccgccaggccccggggtcagangagatgaactgctcggcgttctcgtcgtcgagctcgggcagcgcaaaccggcagctcgtcatcaag

SEQ ID NO: 163 - DNA, 401 bp, Zea mays
  source 1..401: mol_type = genomic DNA
gacatctgtggacttcgtgaacggctcctcgtcctcctcctccatggatcacgccgccggcatgcctaacggcaacggctttagccaacaaggcatgggcgcggccttcatggacgtgacggcgggaatgtggagcaccgcagccgtggattctggcatgtgtgcagggatggaagtggctcagccaccgccgccgccgcmgcagcagccgctgccaccacaagggctggtgcagggggnggtgctcgtcggccgacaaactgtgatgaacggtggcggtgcagttgctgacaagggcatggacatgatggacgtctcttcrgtgtatggcggcgccggtgcaatggcgttcgatctggagctgatggagtcatgcncgttgttttgcggcagcaccggca

SEQ ID NO: 164 - DNA, 401 bp, Zea mays
  source 1..401: mol_type = genomic DNA
tgaaaccagtacgaagaataatgaagttgagaaaaagaagacactgatattacatttgaagaagcgttcaaccaaggaattrtcagagaacactaaaccctcgaagtcagaatttgttgaactttcagaagagaaaattgtaaaacatggaagtgttttgaaactcaagaaacatccacatcgtatggacttgtctccaartaaatctaaaaccaggaggcaaagtagycagagagagagtaaaagatctggcacaaaaaagattaaatattccatgtctgatgatgacagtgtttcctcatcagaaccatcgacatctcttgataacagtgaatcaccaccaaaaagaaaacactcagatggaaaagcaccttcatccagtaccaagaaagggaaaaaga

SEQ ID NO: 165 - DNA, 401 bp, Zea mays
  source 1..401: mol_type = genomic DNA
atggtatggttcttccacatgcttccgctggcattcctaataggcctcatgatggaacagagtcttctagaaatggtacaattgaggatatatgtgctgatgaatcagccaatcatagtggtcacaatttagaaatgcgaaaggatacrgacagtaaatcccatgaaaaggagtcacataagcgagaacaagtcaagaagrtattatcagtatgttctggtgatgagaccaatataatgaaagatgatcaagtagtcagagaaaatgtttcagcttccaaaaaaggtgaagatgagacatctgatttgccagcagaaaaggatgataccatacttccagtcaccagagcttgtacaatgatcaggaccaatcaagatcatacagatgaaagkaaacagcat

SEQ ID NO: 166 - DNA, 401 bp, Zea mays
  source 1..401: mol_type = genomic DNA
tggaggtttaaaaccaaacaagttacctcattggcttcaggaggctgtacgtgctccttcatctaagccacctgaacgtgagctaccagcaacagtttcggcaattgctcagtcggtstgccttcttcttggagaacaagaaccatcaatacctccattcttgatcccgggaacmcctctttctygcccaaaagatccaakgattaattcaaagaagaaactccgcaaggctcagcaatctaactctcatgtasagcattcaaaaattggatctggtgagggtgattgtgtcrctaccccagcacccccatccattgaagcttcacctgcacttcctgtcgattgcgatgatggtgagccttcactcaacttgaaatctgcatcrttgtcatcttcagctg

SEQ ID NO: 167 - DNA, 401 bp, Zea mays
  source 1..401: mol_type = genomic DNA
ctccttcatctaagccacctgaacgtgagctaccagcaacagtttcggcaattgctcagtcggtstgccttcttcttggagaacaagaaccatcaatacctccattcttgatcccgggaacmcctctttctygcccaaaagatccaakgattaattcaaagaagaaactccgcaaggctcagcaatctaactctcatgtasagcattcaaaaattggatctggtgagggtgattgtgtcrctaccccagcacccccatccattgaagcttcacctgcacttcctgtcgattgcgatgatggtgagccttcactcaacttgaaatctgcatcrttgtcatcttcagctggnagcaaggggctagatgarttacctcccrcatttaaagaagcaaaycaaacc

SEQ ID NO: 168 - DNA, 401 bp, Zea mays
  source 1..401: mol_type = genomic DNA
ctcagcaatctaactctcatgtasagcattcaaaaattggatctggtgagggtgattgtgtcrctaccccagcacccccatccattgaagcttcacctgcacttcctgtcgattgcgatgatggtgagccttcactcaacttgaaatctgcatcrttgtcatcttcagctggnagcaaggggctagatgarttacctcccrcatttaaagaagcaaaycaaaccgtggatggctcagaagctgttgcggccaagtcagatgctcctgaaattggttgtcaggtacctggttcctctccattacatgacaaaggtagygaatcctcggggtcgccagtgaaggacactcctgasgctggaatcagattgccaggatcagataactcagytatggcattttct

SEQ ID NO: 169 - DNA, 401 bp, Zea mays
  source 1..401: mol_type = genomic DNA
tcrttgtcatcttcagctggnagcaaggggctagatgarttacctcccrcatttaaagaagcaaaycaaaccgtggatggctcagaagctgttgcggccaagtcagatgctcctgaaattggttgtcaggtacctggttcctctccattacatgacaaaggtagygaatcctcggggtcgccagtgaaggacactcctgasgctggaatcagattgccaggatcagataactcagytatggcattttctgcactcccacttgttgatgaagcacctgggacaagcagcagagcagctgacacgcctgtgccatgtgatggcaatgagtcgaaagaggatgttccactagacaacgctgaaagcaccaggaaactggagccgaacggatgaactggctcctc

SEQ ID NO: 170 - DNA, 401 bp, Zea mays
  source 1..401: mol_type = genomic DNA
tttccatttttgcatatcgttgttggagttagccttagtgactcagtaaatgccacaaaaaacaattctgaaactaagcactattatacctttagtcctttaccaaatagaaagttaaaatgagaatgcactcatcaaggttcagtaaaacttaactccatttcttgttgggaggccgtccatcyaggttcgtaagcttcsaaaaacccaagctttgccttgtttgggaggtcatgtggatggacaaatggaaccccttgtggccatccaatttcagctctagtcttctcacacggactatccaacgaccagatgtggctttcacattctctgagattacaaaatctcagcttgatgcctttcttatctgcaatgtcctgaacacgctccttcaggaccct

SEQ ID NO: 171 - DNA, 401 bp, Zea mays
  source 1..401: mol_type = genomic DNA
tccctggccatcaccagttcaccgctccactccantaccgccgccaccccttcaacacaacctccgcccccggccgtaggggagggagggccggaggaagcgaccgagcaacccancgccggagatgcagatcttcgtgaagacgctgacggggaagaccatcacgctggaggtcgagagtagcgacaccgtcgccaacgwcaaggcgaagatacaggacaaggaaggtagtactaactagcagcagccaccctgttcctctcctatccgctgttccgaagtcgaaagcttgatccttttcagcgtctcctatctgctgcttcaaagtcgaaagcttgctcctttctatggtctctgtaggcagttacttgaacatcgatgtgcatgtcagcgtttggttg

SEQ ID NO: 172 - DNA, 401 bp, Zea mays
  source 1..401: mol_type = genomic DNA
tcgccgtataattaaactaccaaaaaaataaatgataaacaaaaaaatctagatgacaaagataaaaaaaatctagatgaaaaaaaattttgtatggacatatcagcaacataaaaaataaaacaaatccaataaagacaaaaaatagtgaaatgtaaactatagggcttgaaaccaagaccacaaggtgaagagccttgygctctaccaactaggctagacaagctttgtgcttatagctttccaataaaacttttaaaacacaaccgagctcggcgccaagatctgtggcgccgagctcggtgccacgtcggcgccacgacgtctaggtatgccagcgtagcacgtacctcggcgccataggctagggcgccgagctgtgttacctcggcgccatagcc

SEQ ID NO: 173 - DNA, 401 bp, Zea mays
  source 1..401: mol_type = genomic DNA
atcacttgatctcacattacatcaacctcttatgaagacaaatagcttgkcattaatsaagatctgaattgttkgcacaacgttcctctttgcttggcgacctctcttaatgataacttccmaaatttctacataatttgaaatttgattgaatatatttttttctcttstgaagrtatccaaattggagccagtccattrtttgttttttctctgrattcactacrcaagtaagcattcgacacaatctatcaagaaagatcgcagagatcaacttaaggcttgaagacatcattaacaataaggagaaatacaagattgaagctgcacccaatgattttccattggtgacatggaaaccttctacggacatctctactgtggcaaaaagtttgtaagtt

SEQ ID NO: 174 - DNA, 401 bp, Zea mays
  source 1..401: mol_type = genomic DNA
ttacatcaacctcttatgaagacaaatagcttgkcattaatsaagatctgaattgttkgcacaacgttcctctttgcttggcgacctctcttaatgataacttccmaaatttctacataatttgaaatttgattgaatatatttttttctcttstgaagrtatccaaattggagccagtccattrtttgttttttctctgrattcactacrcaagtaagcattcgacacaatctatcaagaaagatcgcagagatcaacttaaggcttgaagacatcattaacaataaggagaaatacaagattgaagctgcacccaatgattttccattggtgacatggaaaccttctacggacatctctactgtggcaaaaagtttgtaagttcccacaacactactyt

SEQ ID NO: 175 - DNA, 401 bp, Zea mays
  source 1..401: mol_type = genomic DNA
gaagaagcaaaacaggatgtgatgcccagtacaacaatgccattattaccaaacttgcttcagtgttgggtgacgccactatcaggcttgcttcgttgttgtggggaacatggcagccctcaagagagcaccaactcatacataaaagaaatgttgcaagtatccaagtttcttcgtgtcatcaatctacaaggcatcgakatcggtgagagcttaccaacarctattgggaatgtrgcacacctkcagtacctckgtgtcactgcttgttccttgaaatatatcccatcgacaattgaaaaccttaaaaawcttcagacgttggatgttcgasacacrtttgtttataaacttccagaagcattttggagtataacgacactgcsgcatgtatttggtga

SEQ ID NO: 176 - DNA, 401 bp, Zea mays
  source 1..401: mol_type = genomic DNA
tgcccagtacaacaatgccattattaccaaacttgcttcagtgttgggtgacgccactatcaggcttgcttcgttgttgtggggaacatggcagccctcaagagagcaccaactcatacataaaagaaatgttgcaagtatccaagtttcttcgtgtcatcaatctacaaggcatcgakatcggtgagagcttaccaacarctattgggaatgtrgcacacctkcagtacctckgtgtcactgcttgttccttgaaatatatcccatcgacaattgaaaaccttaaaaawcttcagacgttggatgttcgasacacrtttgtttataaacttccagaagcattttggagtataacgacactgcsgcatgtatttggtgagggtcttttcttgcctaagcag

SEQ ID NO: 177 - DNA, 401 bp, Zea mays
  source 1..401: mol_type = genomic DNA
actcatacataaaagaaatgttgcaagtatccaagtttcttcgtgtcatcaatctacaaggcatcgakatcggtgagagcttaccaacarctattgggaatgtrgcacacctkcagtacctckgtgtcactgcttgttccttgaaatatatcccatcgacaattgaaaaccttaaaaawcttcagacgttggatgttcgasacacrtttgtttataaacttccagaagcattttggagtataacgacactgcsgcatgtatttggtgagggtcttttcttgcctaagcaggtgggtgacttgaaacacctgcagacacttgaaagcatagatcctgataaggataatggttgggacagcagcacttttgagaaaatggctcaccttcagtcccttcatgtt

SEQ ID NO: 178 - DNA, 401 bp, Zea mays
  source 1..401: mol_type = genomic DNA
aaacttccagaagcattttggagtataacgacactgcsgcatgtatttggtgagggtcttttcttgcctaagcaggtgggtgacttgaaacacctgcagacacttgaaagcatagatcctgataaggataatggttgggacagcagcacttttgagaaaatggctcaccttcagtcccttcatgtttgggattctaagkasaatggagttaatgctgaggctctatctgctgttattgataaagcyaatttcctcgaacacctagataccctcactcttgatgttcatgacatcccattgagtgtgttcaccagttcttccctgaggcgtctycgtaccctaawcttagacggcaagttggacatgttggtgctgcaatctaaagaaaaggagtcaaaatt

SEQ ID NO: 179 - DNA, 401 bp, Zea mays
  source 1..401: mol_type = genomic DNA
gataaagcyaatttcctcgaacacctagataccctcactcttgatgttcatgacatcccattgagtgtgttcaccagttcttccctgaggcgtctycgtaccctaawcttagacggcaagttggacatgttggtgctgcaatctaaagaaaaggagtcaaaatttcatttccccaacmttagcytcctatcattggcaaamacagaggtgtcgcagaacttyatcaacaagcttgggaagctgccttcgctcaccactctaatattggatrtaaaatcctacaaggatgatcaagatcaacttgccttccaagctggtggatttacaagccttacaaagctgakactttctgatctggaaattgtgaaactcgagataaagaaatatgcacttccagaact

SEQ ID NO: 180 - DNA, 401 bp, Zea mays
  source 1..401: mol_type = genomic DNA
tcaccagttcttccctgaggcgtctycgtaccctaawcttagacggcaagttggacatgttggtgctgcaatctaaagaaaaggagtcaaaatttcatttccccaacmttagcytcctatcattggcaaamacagaggtgtcgcagaacttyatcaacaagcttgggaagctgccttcgctcaccactctaatattggatrtaaaatcctacaaggatgatcaagatcaacttgccttccaagctggtggatttacaagccttacaaagctgakactttctgatctggaaattgtgaaactcgagataaagaaatatgcacttccagaactcacagatttngaggttgagtyktactacgacgamatcgaaatcgaggtatatggygagcatgagtttgtg

SEQ ID NO: 181 - DNA, 401 bp, Zea mays
  source 1..401: mol_type = genomic DNA
taaagaaaaggagtcaaaatttcatttccccaacmttagcytcctatcattggcaaamacagaggtgtcgcagaacttyatcaacaagcttgggaagctgccttcgctcaccactctaatattggatrtaaaatcctacaaggatgatcaagatcaacttgccttccaagctggtggatttacaagccttacaaagctgakactttctgatctggaaattgtgaaactcgagataaagaaatatgcacttccagaactcacagatttngaggttgagtyktactacgacgamatcgaaatcgaggtatatggygagcatgagtttgtgaagaaaattgaacaagtggatgaatacctytatggccgtatcactcaagttccggttgctccaaagaaaattg

SEQ ID NO: 182 - DNA, 401 bp, Zea mays
  source 1..401: mol_type = genomic DNA
atcaacaagcttgggaagctgccttcgctcaccactctaatattggatrtaaaatcctacaaggatgatcaagatcaacttgccttccaagctggtggatttacaagccttacaaagctgakactttctgatctggaaattgtgaaactcgagataaagaaatatgcacttccagaactcacagatttngaggttgagtyktactacgacgamatcgaaatcgaggtatatggygagcatgagtttgtgaagaaaattgaacaagtggatgaatacctytatggccgtatcactcaagttccggttgctccaaagaaaattggtcagcttcctagcatgaagcaacgcgtggctagctgaagatcacaggtaaactaagacaccaatatagtacyacttgc

SEQ ID NO: 183 - DNA, 401 bp, Zea mays
  source 1..401: mol_type = genomic DNA
gggaagctgccttcgctcaccactctaatattggatrtaaaatcctacaaggatgatcaagatcaacttgccttccaagctggtggatttacaagccttacaaagctgakactttctgatctggaaattgtgaaactcgagataaagaaatatgcacttccagaactcacagatttngaggttgagtyktactacgacgamatcgaaatcgaggtatatggygagcatgagtttgtgaagaaaattgaacaagtggatgaatacctytatggccgtatcactcaagttccggttgctccaaagaaaattggtcagcttcctagcatgaagcaacgcgtggctagctgaagatcacaggtaaactaagacaccaatatagtacyacttgctkttatttcgat

SEQ ID NO: 184 - DNA, 401 bp, Zea mays
  source 1..401: mol_type = genomic DNA
tttgggagccgcatgtatttgagcatgcgtagagccggccgctccccgcgtttgtcgtccagcctctgcatactcttgcctccctccctagctccgtccagttggccaagccagatcgagcaagcaaaacttttatgacatctcccgttgttgacgcggccgggcttgtcggacgtacgtcctccgatccgttgacggtcnggcccttctgctgggtcgtcctgctgcatgcgtcgccgtggtcggggacacagaggaggagatcgagggngatggagtgcctgctggggctgctcaaggttagggtggtgcgaggagtgcacctggccatctgcgaccccctcacccacagcagcgacccctacgtcgtcctccgccacggacagcaggtaaactgcatg

SEQ ID NO: 185 - DNA, 401 bp, Zea mays
  source 1..401: mol_type = genomic DNA
ccacggccacgcccacgacggccacgagctcgaacgcgttcaggtgctgctcngcgtacagcggccacggcgccagcggcacgccgtgccacaggctctccatcgtcgagttccacccgcagtgggtcacgaagccgcccacggccgggtgggcgagcaccgccttctgcggcgcccaccgcggccacacgaggcccctgyccctggtccgctccaggaaccccgcrgggaggagctcgtccaggtcggcgtccgacgggtgccgcgakccggcmgcgggaggcccgcgcagcgcccacaggaaccggtgcccgctccgctccagccccgccgcggcctcgcgcgccytcgccgcgtcgaaccagcccaggctcccgaagcagaggaacaccaccgacgca

SEQ ID NO: 186 - DNA, 401 bp, Zea mays
  source 1..401: mol_type = genomic DNA
cgccgagctcccgcgccgcggcctccacgtgcggcctgtggagctccaggtaccgcgacttgaactcctgcaggttgccggcgcagtccgccgggggctccacggccgggaggtggtggaaccggacgccagcaatgccggactccgcgacgcgccggacgtgcgcgcccacctcggcggccgactccggcgtcggcggcygcacgacgaggacggtcaccgtgaactcccggcccaggccgtccccgccgcccgcgccgtggcccagcaggcgcttcccggcttcgatgagcgacatgaggtggccggagcccggctcagggaggagcagcacgttgaggctcrccatatctcaccctcckmtccttcctgtgctgacaaaagcttgtgcngctggcgct

SEQ ID NO: 187 - DNA, 401 bp, Zea mays
  source 1..401: mol_type = genomic DNA
ggggtccggccacctcatgtccatgctcgagtcctgtaagcgcgtcctcctcagcagcagcggcggcaaggccttctccatcaccctgctcgtgatgcgcccgccgacggccgaggccacctccgaggtggaggcgcacgtgcggcgcgaggccgcgtgcgggctggacatccgcttccaccgcctccccgccgtggaggyccccaccgacgccgtcggggtcgaggagttcatcgcgcgctacatcggcctccacgcgccgcacgtccgtgacgccgtggccgccatggcgtgccccgtggccgcgctcgtgctcgacctcttcgccgcgccgctggtggacgtggcgcggggcctcggggtgccgtcctacgtcttcatgtcgtccacgggggccatgc

SEQ ID NO: 188 - DNA, 401 bp, Zea mays
  source 1..401: mol_type = genomic DNA
ttagagaagaaattggcatccttksagttcatatacatggtaatttatttgatcataatatgttcaatctgttgagagttttttcttgccatagcaagagatacatgcatcagttcacattacctgatgactatttgtgcaggctacaattttacgatgatcatccttgcacaagaaggtccnagcagtgaaccaaggccragtgaatataccaagactatttgttcggggcttcggatgaaataaaaaaaagaaaaccatttaataaatgatgaaaatcttctatataagtatcagagagaaagtaacaaagtcaatgagtgcttacagatttagcagaccatgtcttactacaagatgtaatatcccacaaactcacttctatacctttcctacaanaa

SEQ ID NO: 189 - DNA, 401 bp, Zea mays
  source 1..401: mol_type = genomic DNA
cagtagcctgaaggaggatataagcctgttgcagaagaagctggagagccagaagaacctgtcrtcagcttacatatctgcacttggtgcttctgaaacmgagaagaaggagctggctagtcggtttsagctyggagagaaagaagccgaggaattacgtgggaagatgagcttgcttgaggaacagatccgtaaagagarggcacggtcatccgagtttgcggtaaaatgtcagaagatggaggaacaratctcctgyagatctcttcttggycaccaaccagtgaaatcatctgyggcgatcaaagatcttcagctcagaaaggtagcctccattaattccatagaacaaaattggcacttcactgccattcatattgtmttattatttgcgaccgatc

SEQ ID NO: 190 - DNA, 401 bp, Zea mays
  source 1..401: mol_type = genomic DNA
tcagaagatggaggaacaratctcctgyagatctcttcttggycaccaaccagtgaaatcatctgyggcgatcaaagatcttcagctcagaaaggtagcctccattaattccatagaacaaaattggcacttcactgccattcatattgtmttattatttgcgaccgatcctggtngtgattggttkgtactagtcccagraagcggtggtgttgctcatgaaatcctacactttttctttacaggagacagagctggccaaggctgcagagaaactggccgactgccagaagacgatagcttcactgagcagccagctgaaatcgttggctgatttcgacgaattcccccctgaaactgagaccagtggcgctgactcagacgacgcctgggacagcgat

SEQ ID NO: 191 - DNA, 401 bp, Zea mays
  source 1..401: mol_type = genomic DNA
tgatacaaagccacccttctgctcgatcgtttgtcgctccatgtttggttcatggtcaactgaaagctgctycgctacaccgttcttgctaataactgcccgtgaatccccaacatttgcaacaactagtttcacgaacttgtcacttcctattaagatggcagtaacagctgtggagccacctcttcccaactctgctgygttttccaatatyttctgatcygtaagttggtatgcrtttcggatagcagtttctggatcactcaagaattctggctgaggaggcaggagtcaataaaataattagcacagttgtcttaaactacattgtsccaccttagacaagatttatagatcaagtcrttttgatgctcttaatgtggctgttgcataraagtgca

SEQ ID NO: 192 - DNA, 401 bp, Zea mays
  source 1..401: mol_type = genomic DNA
cccactaaggctggttcggttagaagtggattgaaaagaattaaatccatagctagttaaaattgaatagaaagatcctccctkatccccatgcaactgaamaaggcttaaactrgaatacttccttagtagcaggagaggatttttttaccttcagagaacacatatcctcangggacctcctcacttcattgcttacakctaaatcagttctccgtttatgaggcaggaccttagtcttaacattgagtatctaggcacaaraacaacaaaaaaaaaaagagaraaattagaacacaacagtaacatgaatcttacaagttcgtctccattgcaattcccttacctgtctggctcttccatcatcaagcttctgcctcttcaccgcttggttcgcttgt

SEQ ID NO: 193 - DNA, 401 bp, Zea mays
  source 1..401: mol_type = genomic DNA
cctcttcaccgcttggttcgcttgtgcaatttcagcgccagcagtggctgcctttccagtcccaaccaaatcacaaccacccctgagaagcyaacaactcagacaagggaaacaatctcaagatttttttagtatgcacatatgaccttgaatcttacttgatcacgctccccttggcattcgtcgaagcagccatagtasctgctctgcacatgttcgcctgcgtctttggcgtgcgcgcaccagtattcactgaagagctgtcacatgtcgaaattggaggkgcaattttkgtagaaggagaaggaggacgcgtctgcatcttaggcgtgctggcagcaccaccrtctgctgcaaagtcagaacttaactawgaataccacaattttccacccaagtaa

SEQ ID NO: 194 - DNA, 401 bp, Zea mays
  source 1..401: mol_type = genomic DNA
tgctgcaaagtcagaacttaactawgaataccacaattttccacccaagtaatcaaagaacacagagkctagcaaatagcaactcacattttacggataggtctgtcgccgagaggaactcgaacgattggggcgatttatccttctcttcttgagccggatgtgaagccggcggcgattccgatattgcmtcwggacatkgagcactgaccaaaacatatggcacacaaacttgcctgaactttccaccatgatgaacccaaaatraattttagtatgatgtgtccgatgcttacccatcaatagcatctgctccgtgtaccgagacgcttcctgctgccacctccacgggyaccgcctgcacaatttacaaatrtataaatcgccagacgagacgaacg

SEQ ID NO: 195 - DNA, 401 bp, Zea mays
  source 1..401: mol_type = genomic DNA
tctgcatcgcagggatcaaacagagttaagatcttgagaaacttgrttttgccacctgcaaagatggcagaaasttgttacttgtyatgggtattactaagcaagctgaaaaatatgtgactctgaaccaaccttgtgagaaggaaccttgccagtgggctcacggtaggagcagccaatggtggcrtattagctaacttmttgtcgcctgctttttcagagggaggttcaggttccttagtgaaaattgacatcgccgtctcctgaagaccttgcaatgaggaatcctttgagctgcgggaaggtaatgtgagccatcatgaggtgagataactgcacaaggtgaatctctctatcgtaagatcgtgagagcatagtgatggtygcacctccctagccgg

SEQ ID NO: 196 - DNA, 401 bp, Zea mays
  source 1..401: mol_type = genomic DNA
tcacggtacattggtacctcaaaatattaatacagtttcttagagagaaaatatgaaggggtcaaattaaaataaacatgaaaatttcaatgaccaaaataaaatttctgcagatttagtaaaacagaaaaaaaaaacaaatycaatgtarctttacctctttcagggaagggagtagcaatgatgagactgcaggtgaarctacagaactagcacgagaggctgactgcccatcaggtaattgccgtaatttgtgamctttctgcgtgtccacatcctcrctaggataaagaacacactttggtttaaaaaaamtttaataataagggcaaagtgtttctatcaatgaaattaaacacaggtaaaggtaagcttgaaatacctttgaacctcttgactat

SEQ ID NO: 197 - DNA, 401 bp, Zea mays
  source 1..401: mol_type = genomic DNA
aygcctnttgataaaaggtctttctcgagcatttccctttcttaacccagcctgcaaagcagcctttgcctgcatgtaccaccatgtacattataattatgcatcgactaataaccacaaacaaaaaactgccaacaaattgwtaatgtgcatcacctctgcaaggttaatggcactatcctcaagaggagcatttgatgytttttcttggatccccagccttgaccttgaagatcgtggagtttcygggtcttcattttgggtcygaacaactgtcccactattagacatatcttcatawgaggagaatctgctgggaggctaagaagtaaagatgtgttagctgctgctcctcagttctatctttgncatgtyttacatagacagtaaggatgttaatc

SEQ ID NO: 198 - DNA, 401 bp, Zea mays
  source 1..401: mol_type = genomic DNA
tgaaacaactcgsacagttcctgtgccctctgatytatcagaaaaatctctgactgcagatttcctaactgttccttgactasagtagaaaaaagaatcaggtaagtgaagacacaccaaaaattacttaatgatgacttaaatatacctcaaagmtggagctgtatcttttgtcctattgaccttgatagtgcctgtacyaaaatcctcctcttcatcatgtgtttgcccattttgtgtggcatccatgcyttttgctgcaaattttggccgctctctaccaataacagtaatagaaggcacacagaatgatataaacattcgagtgcatcaatttactcaattttcactaagaaakaataaccacaaaccttattcgctccaaaagctttggagtcttt

SEQ ID NO: 199 - DNA, 401 bp, Zea mays
  source 1..401: mol_type = genomic DNA
aaattaaaaaaaatatgaagtcaatattttttagtcaaaatgaaatttcgctacagtaaaaacgaaaaggtaatccctgctctgtttatgtggcatgtgtgctactcataatgcagcgactatcctgcaggttataatggaagctatgggaaccccaaggccaattatgctgcaaacccttataactctggatatggcatkaatcaagtatkycmtctggcctgtactttatcatctgcagaactagtcctcacatcccttctcatttgaagctgtttctstattgcaggcaactgctgccactgattctggttctcagtatggaactggtgcaacgcatggttcttggggtgcctatgacttgcaaagggcgwccgggcgtagataagtactgcgaattc

SEQ ID NO: 200 - DNA, 401 bp, Zea mays
  source 1..401: mol_type = genomic DNA
aaacccttataactctggatatggcatkaatcaagtatkycmtctggcctgtactttatcatctgcagaactagtcctcacatcccttctcatttgaagctgtttctstattgcaggcaactgctgccactgattctggttctcagtatggaactggtgcaacgcatggttcttggggtgcctatgacttgcaaagggcgwccgggcgtagataagtactgcgaattcgtcaggtctttcacggaagctttgsccatgcctckatgattgttgtggtggatcagatgttgaacaggtggggcacaggtatagatgaatagcgcacacatttgctttagtttagcttctatcrgcctgtgcaaggsaacatatatctgttgcatcgagtgtcaatgtattgg

SEQ ID NO: 201 - DNA, 401 bp, Zea mays
  source 1..401: mol_type = genomic DNA
ctgcagaactagtcctcacatcccttctcatttgaagctgtttctstattgcaggcaactgctgccactgattctggttctcagtatggaactggtgcaacgcatggttcttggggtgcctatgacttgcaaagggcgwccgggcgtagataagtactgcgaattcgtcaggtctttcacggaagctttgsccatgcctckatgattgttgtggtggatcagatgttgaacaggtggggcacaggtatagatgaatagcgcacacatttgctttagtttagcttctatcrgcctgtgcaaggsaacatatatctgttgcatcgagtgtcaatgtattggaagttttctatcgagcatgaatataytaaggatacgagcstcgtgatgtttmactaattatt

SEQ ID NO: 202 - DNA, 401 bp, Zea mays
  source 1..401: mol_type = genomic DNA
tcaagaaaaaattaaccagaaaattaaggtaaaaaaacgaagttaacgcgaggccagcgggagtgcgggacagmcasacggctagaagaacgtsgcaatggargatccgcagttggtgaggtctggccacgccgcggcagngccrccaataccgccgttgccccagtacatgaasgaggaggggacggttggcgccgtggskgctgtggcggagctaaccccggggttcatggacgcggcggcggcggccatgagcgcgtcagatgcgatgacgttggcgtcgacgttggacgacgacggcgtcggcagctcccagctggcgctctgctggtccccgggcgcgggcttctgctggtgatccccggcaccrgcgacctccgccttgacgtgcagctcctcca

SEQ ID NO: 203 - DNA, 401 bp, Zea mays
  source 1..401: mol_type = genomic DNA
cgrcanartagttcttttgctgcatcataaactgtcaatataaaactaatagtgtgactttatcatcaccttcgttaacactagcaattccaaccttcaagccattaacagcagacataactgcagatttcaatatgagaaacaagattgggttatttatagataagngaagcttttgtagaatcttgttactaggaaagmaaatgtgcagatcacccaagtaggtgaagagcaaatatacaatgaacaatgcatcagaaagacatgaacctgacattgctgatgtgacaacaccatctgctattaccatcgaagtaccaaaaagaacaagcatcaaaagcagcttttttagcatagangaagtctcaagtcgctccttgatcttcaaagatctctcgagc

SEQ ID NO: 204 - DNA, 401 bp, Zea mays
  source 1..401: mol_type = genomic DNA
ttcggggtgcttttctttgccaaccaggcaaccagggccascagaaacactctgamtacgtggggcccgcaaggccagacggtttacgtggggtttatccacatccacctgttgcatgcgcgttgggatgtgcgtggcagggcttccttccgtggcgaaattggccgtggcaggaggtggacagcacggcatttgggctarcttcgaacctctggtttatgagcaggttgtgttgggctggcctgatggtctgtttggttctctgtataaaacgtcacaacttaccctaatttttctgttaaaaattttcaattcaaacgataaagcttagataaattatgacgtttataggaaactaaacaagacgcctgcagctgactctaaacattgacggaggacaa

SEQ ID NO: 205 - DNA, 401 bp, Zea mays
  source 1..401: mol_type = genomic DNA
tgatgacakgagcttcaaaaatcaagttaaaaaaaccatgtccaagaattcacaggatcgaagttgcttattacagaaagagcgaattaagaaacccattcccaggaattcagaggacaatttaagttcctcattccacaaagagcgacctaagaaactgatctccagacattcgaaggacgacccagactccccatctgkcaaagagcaagttaggaaagtcatttcaaaaaatttagaaaatgatgaaanctccccgttccacaaacatcaagtcaagaagtccactccacagagntcaaaggacgacttcttatcccagaaaaaacacattaagccaattggcagaaatggagaagatccaggtttttcatcccgcaaaaagcccattgaggatccag

SEQ ID NO: 206 - DNA, 401 bp, Zea mays
  source 1..401: mol_type = genomic DNA
aaagtacaggggctctttacaaaggttgccacgatgaaaggctacgggcgatcctcggccgttggatcaacgaccgacgcgccagattaaatctaaatttaagcgaaccggtacgcagtaagggacgcacgattgacaaccaacgatccagatttaaaaagtcttgcgatcaatccccctccacccgattgctgatcaacrgtcaggatgagattcactgaagcggtacacccgatctaatcgtagccctctgttcccgatccaaaggccagggcagccccccattccctctgatggaggagcacggtggcgcgcgcccgaccacggcggtccgcccgccggagatctcgaatccggccagtacgaacgccataacccgattcactcatcccacgcgcaat

SEQ ID NO: 207 - DNA, 401 bp, Zea mays
  source 1..401: mol_type = genomic DNA
gtgaaccccccagtaattcccctctgccgtgcggctggagcgataggctcgccggtgaaccaccgcctcccggccttgcgccgcgtggctctgcctttccaccgcgtcgtcgtgactccctattgctgtctcatcaacgccggtgaggttgccacgacgggggacacaaaatcgcgacgatratgttcttccttatctccrgccacccgcgctrcctggtcaaattaagtgccaccgtcgaagcctcctataaattgagccygtgactagcttcgcaacatcatcacccactcagccaccgctaatcgctagcaatcattcgccagagctcgcgaattttgaattcgccccaaatcwgtctccgccgccgtgaatcacaccctgycatggccagccctccc

SEQ ID NO: 208 - DNA, 401 bp, Zea mays
  source 1..401: mol_type = genomic DNA
caagcatgtcgctgtagtgcttgagcgactcctgccgctgcagccgcatctccatgttgaacttgtggatgaacgcctccacgcggcggttaagctcgtcctgccccagngacggctcccgcngcaccggcgcaggaggcgccttgctccttccggcgcscccaccgtcgttgaaagtctcggccttccgcatcgcccccsccggcgcgrccgccgctggatcgacctycccgctgcggccgccggcggacggacggcggcccgggcgggcgtcccaggtgtcggacttcttgaggtgycgagctagcggcgggccccggccctccgtgatggccttccacgtgctctccagcgtctcctccttccgcggccgcgctacccgcagagccctgctacctgcg

SEQ ID NO: 209 - DNA, 121 bp, Zea mays
  source 1..121: mol_type = genomic DNA
ctccacggcaaaagggctctttcccttgtttgttgtacacatgcatgttctccaatttctsgaagagggacgagtccaaagctggtctatcctgatcgacaaagagtggtcccagttcatc